(19) United States
(12) Patent Application Publication
(10) Pub. No.: US 2024/0101695 A1
(43) Pub. Date: Mar. 28, 2024

(54) IMMUNOGLOBULIN E ANTIBODY COMPOSITIONS AND METHODS OF USE

(71) Applicant: The Regents of the University of California, Oakland, CA (US)

(72) Inventors: Manuel L. PENICHET, Los Angeles, CA (US); Pierre V. CANDELARIA, Los Angeles, CA (US); Miguel NAVA, Los Angeles, CA (US); Tracy R. WELLS, Los Angeles, CA (US)

(73) Assignee: The Regents of the University of California, Oakland, CA (US)

(21) Appl. No.: 18/043,554

(22) PCT Filed: Sep. 1, 2021
(86) PCT No.: PCT/US2021/048714

§ 371 Date: Feb. 28, 2023

Related U.S. Application Data
(60) Provisional application No. 63/073,292, filed on Sep. 1, 2020.

(51) Int. Cl. C07K 16/28; A61K 45/06; A61P 35/00; C12N 15/63; G01N 33/574

(52) U.S. Cl. CPC C07K 16/2896; A61K 45/06; A61P 35/00; C12N 15/63; G01N 33/574

(57) ABSTRACT

Aspects of the disclosure relate to antibodies and methods of use. Various antibodies are disclosed, including engineered antibodies targeting CD138, CD38, and TfR1. Certain aspects are directed to IgE antibodies and use in diagnosis and/or treatment of cancer. Also disclosed are kits and pharmaceutical compositions comprising one or more engineered antibodies.

### CROSS REFERENCE TO RELATED APPLICATIONS

This application a national phase application under 35 U.S.C. § 371 of International Application No. PCT/US2021/048714, filed Sep. 1, 2021, which claims priority to U.S. Provisional Patent Application No. 63/073,292, filed Sep. 1, 2020, all of which are incorporated herein by reference in their entirety.

This invention was made with government support under Grant Numbers CA196266 and CA181115, awarded by the National Institutes of Health and under Grant No. W81XWH-20-1-0455, awarded by the Department of Defense. The government has certain rights in the invention.

### SEQUENCE LISTING

The instant application contains a Sequence Listing which has been submitted in ASCII format and is hereby incorporated by reference in its entirety. Said ASCII copy, created on Aug. 30, 2021, is named UCLA_P0123WO_Sequence Listing.txt and is 34,081 bytes in size.

### BACKGROUND

### I. Field of the Invention

Aspects of this invention relate to at least the fields of immunology, cancer biology, and medicine.

### II. Background

IgE is a highly conserved immunoglobulin (Ig) class found in all mammals¹, which similar to its IgG counterpart, is affinity matured². IgE antibodies are heterotetramers composed of two heavy and two light chains (H₂L₂)^{3,4}. There are two FcεRs, FcεRI that binds IgE with high affinity (Kₐ=10¹⁰ M⁻¹) and is expressed on human mast cells, monocytes, macrophages, eosinophils, basophils, Langerhans cells, and dendritic cells, and FcεRII (CD23), which in its trimeric form binds IgE with lower, but still high affinity (Kₐ=10⁸ M⁻¹) that is expressed on human eosinophils, monocytes, macrophages, and dendritic cells³⁻⁵. Thus, IgE binds to FcεRs with extremely high affinity, which in the case of FcεRI is two-three orders of magnitude higher than that of IgG for the FcεRs (FcεRI-III) and is considered to be a cytophilic antibody³⁻⁷. IgE antibodies are known as mediators of allergic reactions, characterized by immediate hypersensitivity (Type I hypersensitivity/anaphylaxis), an acute inflammatory response involving the release of histamine that increases vascular (capillary) permeability. The uniqueness of this reaction is due to the presence of mast cells in the tissue that are sensitized by IgE bound to FcεRI^{3,4,8}.

Multiple epidemiological studies on the association of allergies with cancer support a lower cancer risk among people with a history of allergies⁹⁻¹². IgE antibodies isolated from pancreatic cancer patients mediate antibody-dependent cell-mediated cytotoxicity (ADCC) against cancer cells¹³ and higher levels of polyclonal IgE in non-allergic individuals are directly correlated with lower disease incidence and higher survival in multiple myeloma (MM)¹⁴. In addition, patients with IgE deficiency have higher frequency of malignancies^{15,16}. Taken together, these results suggest a role of IgE in cancer immunosurveillance.

### SUMMARY

The present disclosure fulfills certain needs in the art, including improved and alternate antibody compositions and use in methods for treatment and/or diagnosis of cancer. The present disclosure is based, at least in part, on the generation of engineered antibodies capable of targeting various tumor antigens, including, for example, CD138, CD38, and transferrin receptor 1 (TfR1). In some embodiments, the disclosed antibodies are IgE antibodies comprising one or more variable regions and/or one or more CDRs derived from antibodies of a different isotype (e.g., IgG). Also disclosed are methods for cancer treatment comprising the use of one or more of the disclosed engineered antibodies.

Embodiments of the present disclosure include antibodies, engineered antibodies, nucleic acids, vectors, polynucleotides, reagents, pharmaceutical compositions, methods for treatment of cancer, methods for diagnosis of cancer, methods for detecting a tumor antigen, methods for detecting CD138, methods for detecting CD38, and methods for detecting TfR1. Compositions of the disclosure can include at least 1, 2, 3, 4, 5, or more of the following elements: an engineered antibody, a nucleic acid, a vector, an excipient, a chemotherapeutic, an immunotherapeutic, a checkpoint inhibitor, and a diagnostic agent. Methods of the disclosure can include at least 1, 2, 3, 4, 5 or more of the following steps: administering an engineered antibody, administering a therapeutic nucleic acid, diagnosing a subject for cancer, treating a subject for cancer, detecting a tumor antigen in a sample from a subject, detecting CD138 in a sample from a subject, detecting CD38 in a sample from a subject, detecting TfR1 in a sample from a subject, generating an engineered antibody, expressing an engineered antibody, introducing a nucleic acid encoding one or more regions of an antibody into a cell, generating a pharmaceutical composition, and engineering an antibody. It is specifically contemplated that one or more of these components and/or steps may be excluded from certain embodiments of the disclosure.

Disclosed herein, in some embodiments, is an antibody comprising (a) a heavy chain variable (V_{H}) region of a CD138-binding antibody; (b) a light chain variable (V_{L}) region of the CD138-binding antibody; and (c) a heavy chain constant (C_{H}) region of an immunoglobulin epsilon heavy chain. In some embodiments, the CD138-binding antibody is B-B4, BC/B-B4, B-B2, DL-101, 1D4, M115, 1.BB.210, 2Q1484, 5F7, 104-9, 281-2, or a Fab fragment (e.g., Fab fragment) or single chain variable fragment (scFv) thereof. In some embodiments, the CD138-binding antibody is B-B4, 1D4, M115, or a Fab fragment or single chain variable fragment (scFv) thereof. In some embodiments, the CD138-binding antibody is B-B4, 1D4, M115, or a Fab fragment or scFv thereof. In some embodiments, the V_{H} region comprises SEQ ID NO:1, SEQ ID NO:2, and SEQ ID NO:3. In some embodiments, the V_{L} region comprises SEQ ID NO:4, SEQ ID NO:5, and SEQ ID NO:6. In some embodiments, the CD138-binding antibody is B-B4 or a Fab fragment or scFv thereof. In some embodiments, the V_{H} region has at least 95% sequence identity with SEQ ID NO:7. In some embodiments, the V_{H} region comprises SEQ ID NO:7. In some embodiments, the V_{L} region has at least 95% sequence identity with SEQ ID NO:8. In some embodiments, the V_{L} region comprises SEQ ID NO:8. In some embodiments, the antibody comprises a sequence having at least 95% identity to SEQ ID NO:27. In some embodiments, the antibody comprises SEQ ID NO: 27. In some embodiments, the antibody comprises a sequence having at least 95% identity to SEQ ID NO:28. In some embodiments, the antibody comprises SEQ ID NO: 28.

Disclosed herein, in some embodiments, is an antibody comprising (a) a V_{H} region of a CD38-binding antibody; (b) a V_{L} region of the CD38-binding antibody; and (c) a C_{H} region of an immunoglobulin epsilon heavy chain. In some embodiments, the V_{H} region comprises SEQ ID NO:11, SEQ ID NO:12, and SEQ ID NO:13. In some embodiments, the V_{L} region comprises SEQ ID NO:14, SEQ ID NO:15, and SEQ ID NO:16. In some embodiments, the CD38-binding antibody is isatuximab, felzartamab, daratumumab, or a Fab fragment or scFv thereof. In some embodiments, the CD38-binding antibody is daratumumab or a Fab fragment or scFv thereof. In some embodiments, the V_{H} region has at least 95% sequence identity with SEQ ID N0:17. In some embodiments, the V_{H} region comprises SEQ ID NO:17. In some embodiments, the V_{L} region has at least 95% sequence identity with SEQ ID NO:18. In some embodiments, the V_{L} region comprises SEQ ID NO:18. In some embodiments, the antibody comprises a sequence having at least 95% identity to SEQ ID NO:29. In some embodiments, the antibody comprises SEQ ID NO. 29. In some embodiments, the antibody comprises a sequence having at least 95% identity to SEQ ID NO:30. In some embodiments, the antibody comprises SEQ ID NO: 30.

Disclosed herein, in some embodiments, is an antibody comprising (a) a V_{H} region of a TfR1-binding antibody; (b) a V_{L} region of the TfR1-binding antibody; and (c) a C_{H} region of an immunoglobulin epsilon heavy chain. In some embodiments, the V_{H} region comprises SEQ ID NO:19, SEQ ID NO:20, and SEQ ID NO:21. In some embodiments, the V_{L} region comprises SEQ ID NO:22, SEQ ID NO:23, and SEQ ID NO:24. In some embodiments, the TfR1-binding antibody is 7579, E2.3, A27.15, B3/25, 43/31, D65.30, A24, RBC4, 42/6, D2C, JST-TFR09, H7, or ch128.1. In some embodiments, the TfR1-binding antibody is ch128.1 or a Fab fragment or scFv thereof. In some embodiments, the V_{H} region has at least 95% sequence identity with SEQ ID NO:25. In some embodiments, the V_{H} region comprises SEQ ID NO:25. In some embodiments, the V_{L} region has at least 95% sequence identity with SEQ ID NO:26. In some embodiments, the V_{L} region comprises SEQ ID NO:26. In some embodiments, the antibody comprises a sequence having at least 95% identity to SEQ ID NO:31. In some embodiments, the antibody comprises SEQ ID NO: 31. In some embodiments, the antibody comprises a sequence having at least 95% identity to SEQ ID NO:32. In some embodiments, the antibody comprises SEQ ID NO: 32.

In some embodiments, the C_{H} region comprises SEQ ID NO:9. In some embodiments, the antibody further comprises a light chain constant (C_{L}) region. In some embodiments, the C_{L} region is a C_{L} region of an immunoglobulin kappa constant chain. In some embodiments, the C_{L} region comprises SEQ ID NO:10. In some embodiments, the C_{L} region is a C_{L} region of an immunoglobulin lambda constant chain.

Also disclosed, in some embodiments, is a nucleic acid encoding any of the antibodies described herein. In some embodiments, disclosed is a vector comprising a nucleic acid encoding any of the antibodies described herein. Also disclosed, in some embodiments, is a pharmaceutical composition comprising (i) any of the antibodies described herein; (ii) a nucleic acid encoding any of the antibodies described herein; or (iii) a vector comprising a nucleic acid encoding any of the antibodies described herein; and a pharmaceutically acceptable excipient. In some embodiments, the pharmaceutical composition further comprises an additional therapeutic. In some embodiments, the additional therapeutic is a chemotherapeutic, a nucleic acid, a protein, or a nanodrug. In some embodiments, the additional therapeutic is a nucleic acid, wherein the nucleic acid is an antisense oligonucleotide, a small interfering RNA (siRNA), a clustered regularly interspaced short palindromic repeats (CRISPR)-based gene therapy, or a viral vector. In some embodiments, the additional therapeutic is a protein, wherein the protein is a toxin or an enzyme. In some embodiments, the additional therapeutic is operatively linked to the antibody.

Disclosed herein, in some embodiments, is a composition comprising an antibody described herein bound to a cancer antigen. In some embodiments, the cancer antigen is a CD38 molecule. In some embodiments, the cancer antigen is a CD138 molecule. In some embodiments, the cancer antigen is a TfR1 molecule. In some embodiments, the cancer antigen is a soluble molecule. In some embodiments, the cancer antigen is attached to the surface of a cancer cell, such that the composition comprises the antibody bound to the cancer cell. The cancer cell may be a previously irradiated cancer cell. In some embodiments, the antibody is further bound to an antigen-presenting cell, such as a dendritic cell, via the Fc domain. For example, the antibody may be bound to an Fcεreceptor (e.g., FcεRI, FcεRII) on a dendritic cell.

Also disclosed, in some embodiments, is a method for treating a subject for cancer, the method comprising administering to the subject an effective amount of any of the antibodies described herein, a nucleic acid encoding any of the antibodies described herein, a vector comprising a nucleic acid encoding any of the antibodies described herein, and/or a composition (e.g., vaccine composition, dendritic cell therapy composition, etc.) described herein. Further disclosed, in some embodiments, is a method for preventing cancer, the method comprising administering to a subject an effective amount of any of the antibodies described herein, a nucleic acid encoding any of the antibodies described herein, a vector comprising a nucleic acid encoding any of the antibodies described herein, and/or a composition (e.g., vaccine composition, dendritic cell therapy composition, etc.) described herein.

In some embodiments, the cancer is multiple myeloma (MM), acute lymphoblastic leukemia (ALL), acute myeloid leukemia (AML), aggressive natural killer (NK) cell leukemia (ANKL), chronic lymphocytic leukemia (CLL), or non-Hodgkin lymphoma (NHL) including NK/T-cell lymphoma and mantle cell lymphoma (MCL). In some embodiments, the cancer is esophageal squamous cell carcinoma, breast cancer, ovarian cancer, lung cancer, cervical cancer, bladder cancer, colorectal cancer, kidney cancer, osteosarcoma, pancreatic cancers, cholangiocarcinoma, renal cell carcinoma, hepatocellular carcinoma^{17,18}, adrenal cortical carcinoma, glioblastoma. In some embodiments, the cancer is breast cancer. In some embodiments, the cancer is triple negative breast cancer (TNBC). In some embodiments, the cancer is HER2/neu positive breast cancer. In some embodiments, the cancer is NHL. In some embodiments, the cancer is MM. In some embodiments, the method further comprises administering an additional cancer therapy to the subject. In some embodiments, the additional cancer therapy is radiotherapy, chemotherapy, or immunotherapy.

Also disclosed, in some embodiments, is a method for diagnosing a subject for cancer comprising providing to the subject any of the antibodies described herein and a diagnostic agent. In some embodiments, the diagnostic agent is a dye.

Also disclosed, in some embodiments, is a kit comprising (i) any of the antibodies described herein, and (ii) instructions for use for detecting a tumor antigen in a biological sample. In some embodiments, the tumor antigen is CD138. In some embodiments, the tumor antigen is CD38. In some embodiments, the tumor antigen is TfR1.

In some embodiments, disclosed herein is an antibody comprising (a) a V_{H} region comprising SEQ ID NO:7; (b) a V_{L} region comprising SEQ ID NO:8; and (c) a C_{H} region comprising SEQ ID NO:9. In some embodiments, disclosed herein is an antibody comprising (a) a V_{H} region comprising SEQ ID NO: 17; (b) a V_{L} region comprising SEQ ID NO:18; and (c) a C_{H} region comprising SEQ ID NO:9. In some embodiments, disclosed herein is an antibody comprising (a) a V_{H} region comprising SEQ ID NO:25; (b) a V_{L} region comprising SEQ ID NO:26; and (c) a C_{H} region comprising SEQ ID NO:9.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive “or”.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

“Individual, “subject,” and “patient” are used interchangeably and can refer to a human or non-human.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A and 1B show sodium dodecyl sulfate-polyacrylamide gel electrophoresis (SDS-PAGE) analysis of the anti-CD38 IgE antibody. Affinity-purified anti-CD38 IgG1 and anti-CD38 IgE were analyzed by SDS-PAGE under non-reduced (FIG. 1A) or reduced (FIG. 1B) conditions. The protein molecular weight (m.w.) marker is indicated on the left.

FIGS. 2A and 2B show SDS-PAGE analysis of the an anti-CD138 IgE antibody. Affinity-purified anti-CD138 IgG1 and anti-CD138 IgE were analyzed by SDS-PAGE under non-reduced (FIG. 2A) or reduced (FIG. 2B) conditions. The protein m.w. marker is indicated on the left.

FIGS. 3A and 3B show SDS-PAGE analysis of the anti-TfR1 IgE antibody. Affinity-purified anti-TfR1 IgG1 and anti-TfR1 IgE were analyzed by SDS-PAGE under non-reduced (FIG. 3A) or reduced (FIG. 3B) conditions. The protein m.w. marker is indicated on the left.

FIG. 4 shows flow cytometry analysis demonstrating binding of the anti-CD38 IgE antibody to CD38 and FcεRI. Human multiple myeloma (MM) cells (MM.1S) expressing CD38 were incubated with either IgE isotype control (non-targeting control), anti-CD38 IgG1, or anti-CD38 IgE. The rat basophilic leukemia cells RBL SX-38 expressing human FcεRI were incubated with either IgE isotype control, anti-CD38 IgG1, or anti-CD38 IgE. Antibody binding was detected using a PE-conjugated goat F(ab′)₂ anti-human 1 antibody. Grey filled histograms represent cells incubated with secondary antibody only (“no antibody”). Empty black line histograms represent cells incubated with primary antibodies (IgE isotype control, anti-CD38 IgG1, or anti-CD38 IgE).

FIG. 5 shows flow cytometry analysis demonstrating binding of the anti-CD138 IgE antibody to CD138 and FcεRI. Human MM cells (MM.1S) expressing CD138 were incubated with either IgE isotype control (non-targeting control), anti-CD138 IgG1, or anti-CD138 IgE. The rat basophilic leukemia cells RBL SX-38 expressing human FcεRI were incubated with either IgE isotype control, anti-CD138 IgG1, or anti-CD138 IgE. Antibody binding was detected using a PE-conjugated goat F(ab′)₂ anti-human κ antibody. Grey line histograms represent cells incubated with secondary antibody only (“no antibody”). Black line histograms represent cells incubated with primary antibodies (IgE isotype control, anti-CD138 IgG1, or anti-CD138 IgE).

FIG. 6 shows flow cytometry analysis demonstrating binding of the anti-CD138 IgE antibody to human breast cancer cells. Human triple negative breast cancer (TNBC) cells (MDA-MB-468) and human HER2/neu positive breast cancer cells (SK-BR-3) expressing CD138 were incubated with either IgE isotype control (non-targeting control) or anti-CD138 IgE. Antibody binding was detected using a PE-conjugated goat F(ab′)₂ anti-human κ antibody. Grey filled histograms represent cells incubated with secondary antibody only (“no antibody”). Empty black line histograms represent cells incubated with primary antibodies (IgE isotype control or anti-CD138 IgE).

FIG. 7 shows flow cytometry analysis demonstrating binding of the anti-TfR1 IgE antibody to TfR1 and FcεRI. Human MM cells (MM.1S) expressing TfR1 were incubated with either IgE isotype control (non-targeting control) or anti-TfR1 IgE. The rat basophilic leukemia cells RBL SX-38 expressing human FcεRI were incubated with either IgE isotype control or anti-TfR1 IgE. Antibody binding was detected using a PE-conjugated mouse IgG1 anti-human IgE antibody. Grey filled histograms represent cells incubated with secondary antibody only (“no antibody”). Empty black line histograms represent cells incubated with primary antibodies (IgE isotype control or anti-TfR1 IgE).

FIG. 8 shows an in vitro degranulation assay demonstrating IgE-mediated degranulation triggered by the anti-CD38 IgE antibody in the presence of human MM cells. Rat basophilic leukemia cells RBL SX-38 expressing human FcεRI were incubated with either IgE isotype control, anti-CD38 IgG1, or anti-CD38 IgE with or without CD38 expressing human MM cells MM.1S. Degranulation releases β-hexosaminidase into the cell culture media, which is measured via an enzymatic colorimetric assay. Percentage (%) of degranulation was determined by comparison to total β-hexosaminidase released after membrane solubilization by 1% Triton X-100. ****p<0.0001 (Student's t-test) compared with either component alone. Error bars represent standard deviations (SD) of triplicate measurements.

FIG. 9 shows an in vitro degranulation assay demonstrating IgE-mediated degranulation triggered by the anti-CD138 IgE antibody in the presence of human MM cells. Rat basophilic leukemia cells RBL SX-38 expressing human FcεRI were incubated with either IgE isotype control, anti-CD138 IgG1, or anti-CD138 IgE with or without CD138 expressing human MM cells MM.1S. Degranulation releases β-hexosaminidase into the cell culture media, which is measured via an enzymatic colorimetric assay. Percentage (%) of degranulation was determined by comparison to total β-hexosaminidase released after membrane solubilization by 1% Triton X-100. ****p<0.0001 (Student's t-test) compared with either component alone. Error bars represent SD of triplicate measurements.

FIG. 10 shows an in vitro degranulation assay demonstrating IgE-mediated degranulation triggered by the anti-CD138 IgE antibody in the presence of human TNBC cells. Rat basophilic leukemia cells RBL SX-38 expressing human FcεRI were incubated with either IgE isotype control, anti-CD138 IgG1, or anti-CD138 IgE with or without CD138 expressing human TNBC cells MDA-MB-468. Degranulation releases β-hexosaminidase into the cell culture media, which is measured via an enzymatic colorimetric assay. Percentage (%) of degranulation was determined by comparison to total β-hexosaminidase released after membrane solubilization by 1% Triton X-100. ****p<0.0001 (Student's t-test) compared with either component alone. Error bars represent SD of triplicate measurements.

FIG. 11 shows an in vitro degranulation assay demonstrating IgE-mediated degranulation triggered by the anti-CD138 IgE antibody in the presence of human HER2/neu positive breast cancer cells. Rat basophilic leukemia cells RBL SX-38 expressing human FcεRI were incubated with either IgE isotype control, anti-CD138 IgG1, or anti-CD138 IgE with or without CD138 expressing human HER2/neu positive breast cancer cells SK-BR-3. Degranulation releases β-hexosaminidase into the cell culture media, which is measured via an enzymatic colorimetric assay. Percentage (%) of degranulation was determined by comparison to total β-hexosaminidase released after membrane solubilization by 1% Triton X-100. ****p<0.0001 (Student's t-test) compared with either component alone. Error bars represent SD of triplicate measurements.

FIG. 12 shows an in vitro degranulation assay demonstrating IgE-mediated degranulation triggered by the anti-TfR1 IgE antibody in the presence of human MM cells. Rat basophilic leukemia cells RBL SX-38 expressing human FcεRI were incubated with either IgE isotype control, anti-TfR1 IgG1, or anti-TfR1 IgE with or without TfR1 expressing human MM cells MM.1S. Degranulation releases β-hexosaminidase into the cell culture media, which is measured via an enzymatic colorimetric assay. Percentage (%) of degranulation was determined by comparison to total β-hexosaminidase released after membrane solubilization by 1% Triton X-100. ****p<0.0001 (Student's t-test) compared with either component alone. Error bars represent SD of triplicate measurements.

FIG. 13 shows a passive cutaneous anaphylaxis (PCA) assay in huFcεRI mice demonstrating IgE-mediated in vivo degranulation triggered by the anti-CD38 IgE antibody. Images of the skin of mice injected intradermically (i.d.) with buffer [phosphate buffered saline (PBS)], anti-CD38 IgG1, or anti-CD38 IgE, followed by an intravenous (i.v.) injection of a goat anti-human c antibody plus 1% Evans blue in PBS are shown. Cutaneous anaphylaxis was assessed visually by the blue dye leakage from blood vessels into the skin due to vasodilation.

FIG. 14 shows a PCA assay in huFcεRI mice demonstrating in vivo IgE-mediated degranulation triggered by the anti-CD138 IgE antibody. Images of the skin of mice injected i.d. with buffer (PBS), anti-CD138 IgG1, or anti-CD138 IgE, followed by an i.v. injection of a goat anti-human c antibody plus 1% Evans blue in PBS are shown. Cutaneous anaphylaxis was assessed visually by the blue dye leakage from blood vessels into the skin due to vasodilation.

FIG. 15 shows a PCA assay in huFcεRI mice demonstrating in vivo IgE-mediated degranulation triggered by the anti-TfR1 IgE antibody. Images of the skin of mice injected i.d. with buffer (PBS), anti-TfR1 IgG1, or anti-TfR1 IgE, followed by an i.v. injection of a goat anti-human c antibody plus 1% Evans blue in PBS are shown. Cutaneous anaphylaxis was assessed visually by the blue dye leakage from blood vessels into the skin due to vasodilation.

FIG. 16 shows three-color flow cytometry analysis demonstrating antibody-dependent cell-mediated phagocytosis (ADCP) triggered by the anti-CD38 IgE antibody against human MM cells in the presence of human monocytes as effector cells. Monocytes were isolated from human peripheral blood mononuclear cells (PBMC) and incubated with interleukin-4 (IL-4). Human MM cells (MM.1S) expressing CD38 were labeled with CFSE and incubated human monocytes (5:1 effector-to-target ratio) in the presence of IgE isotype control or anti-CD38 IgE. Cells were washed and incubated with PE-conjugated anti-human CD89 antibody to label monocytes and with DAPI to identify dead cells. CFSE⁺/PE⁺ cells designate the occurrence of ADCP and CFSE⁺/DAPI⁺ cells designate the occurrence of antibody-dependent cell-mediated cytotoxicity (ADCC). Cells treated with saponin were used as positive control for dead cells (100% killing). The error bars indicate SD of quadruplicate samples. ***p<0.001 (Student's t-test) compared to IgE isotype control.

FIG. 17 shows three-color flow cytometry analysis demonstrating ADCC and ADCP triggered by the anti-CD38 IgE antibody against human MM cells in the presence of human M1 macrophages as effector cells. Human monocytes obtained from PBMC were differentiated towards M1 activated macrophages. Human MM cells (MM.1S) expressing CD38 were labeled with CFSE and incubated with human M1 activated macrophages (5:1 effector-to-target ratio) in the presence of IgE isotype control or anti-CD38 IgE. Cells were washed and incubated with PE-conjugated anti-human CD89 antibody to label monocytes and with DAPI to identify dead cells. CFSE⁺/PE⁺ cells designate the occurrence of ADCP and CFSE⁺/DAPI⁺ cells designate the occurrence of ADCC. Cells treated with saponin were used as positive control for dead cells (100% killing). The error bars indicate SD of quadruplicate samples. ***p<0.001 and *p<0.05 (Student's t-test) compared to IgE isotype control.

FIG. 18 shows three-color flow cytometry analysis demonstrating ADCP triggered by the anti-CD138 IgE antibody against human MM cells in the presence of human monocytes as effector cells. Monocytes were isolated from human PBMC and incubated with IL-4. Human MM cells (MM.1S) expressing CD138 were labeled with CFSE and incubated human monocytes (5:1 effector-to-target ratio) in the presence of IgE isotype control or anti-CD138 IgE. Cells were washed and incubated with PE-conjugated anti-human CD89 antibody to label monocytes and with DAPI to identify dead cells. CFSE⁺/PE⁺ cells designate the occurrence of ADCP and CFSE⁺/DAPI⁺ cells designate the occurrence of ADCC. Cells treated with saponin were used as positive control for dead cells (100% killing). The error bars indicate SD of quadruplicate samples. ****p<0.0001 (Student's t-test) compared to IgE isotype control.

FIGS. 19A and 19B shows Kaplan-Meier survival analysis (FIG. 19A) demonstrating in vivo anti-tumor activity triggered by the anti-CD38 IgE antibody against disseminated human MM cells in C.B-17 severe combined immune deficiency (SCID)-Beige mice in the presence of human PBMC. C.B-17 SCID-Beige female mice (8-12 weeks old) exposed to a total-body sublethal irradiation (Day −1) were implanted with 5×10⁶ MM.1S human MM cells i.v. via the tail vein. On Day 1 and Day 7 post-implant, mice were treated (via tail vein injection) with buffer (PBS) control, 100 μg of anti-CD38 IgE, 5×10⁶ PBMC, or 5×10⁶ PBMC combined with 100 μg of anti-CD38 IgE. Mice were then observed for the onset of hind-limb paralysis (end point) and the number of days survived recorded. (FIG. 19B) shows the numbers of animals per group indicated in parentheses in the left hand column of the table, which also shows the median survival and the p-values (log-rank test) comparing the anti-CD38 IgE+PBMC treatment regimens with the different control groups and resulting in significant (p<0.05) increase in survival.

### DETAILED DESCRIPTION OF THE INVENTION

Aspects of the present disclosure are directed to IgE antibodies, including engineered IgE antibodies, and methods of use. In some embodiments, disclosed are antibodies comprising a variable region (e.g., V_{H} and/or V_{L} domain) from an IgG antibody (or variant thereof) and a constant region from an IgE antibody. In some embodiments, disclosed is an engineered antibody comprising a V_{H} domain of a CD138-, CD38-, or TfR1-binding antibody. Also disclosed are methods of use of engineered antibodies for diagnosis, prevention, and/or treatment of a subject with cancer.

### I. Antibodies

Aspects of the disclosure relate to antibodies that specifically bind to CD38, CD138, or TfR1. In some embodiments, the disclosed antibodies are mouse, chimeric, humanized, or fully human anti-CD38, anti-CD138, or anti-TfR1 antibodies.

The term “antibody” refers to an intact immunoglobulin of any class or isotype, or a fragment thereof that can compete with the intact antibody for specific binding to the target antigen, and includes chimeric, humanized, and fully human antibodies. Also contemplated are antibodies having specificity for more than one antigen or target, including bispecific antibodies, trispecific antibodies, tetraspecific antibodies, and other multispecific antibodies. As used herein, the terms “antibody” or “immunoglobulin” are used interchangeably and refer to any of several classes of structurally related proteins that function as part of the immune response of an animal, including IgM, IgD, IgG, IgA, IgE, and related proteins, as well as polypeptides comprising antibody complementarity-determining regions (CDRs) that retain antigen-binding activity. Antibodies from various species are contemplated, including but not limited to human, mouse, goat, horse, rabbit, donkey, bovine, canine, chicken, feline, guinea pig, hamster, monkey, rat, sheep, and pig antibodies.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable of binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen would recognize an epitope on the target antigen within a complex mixture.

The epitope regions of a given polypeptide can be identified using many different epitope mapping techniques well known in the art, including: x-ray crystallography, nuclear magnetic resonance spectroscopy, site-directed mutagenesis mapping, protein display arrays, and hydrogen-deuterium exchange see, e.g., Rockberg and Nilvebrant (Eds.), Epitope Mapping Protocols, Humana Press, New York, NY, USA (2018). Such techniques are known in the art and described in, e.g., U.S. Pat. No. 4,708,871; Geysen et al., Proc. Natl. Acad. Sci., USA 81(13):3998-4002 (1984); Geysen et al., Proc. Natl. Acad. Sci., USA 82:178-182 (1985); and Geysen et al., Mol. Immunol., 23(7):709-715 (1986), each of which is incorporated by reference herein in their entirety. Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule.

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, for chimeric antibodies, the variable regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front. Immunol., 4: Article 302 (2013)).

The term “light chain” may describe a full-length light chain or fragments thereof. A full-length light chain has a molecular weight of around 25,000 Daltons and includes a variable region domain (abbreviated herein as V_{L}), and a constant region domain (abbreviated herein as C_{L}). There are two classifications of light chains, identified as kappa (κ) and lambda (λ). The term “V_{L} fragment” means a fragment of the light chain of a monoclonal antibody that includes all or part of the light chain variable region, including CDRs. A V_{L} fragment can further include light chain constant region sequences. The variable region domain of the light chain is at the amino-terminus of the polypeptide.

The term “heavy chain” may describe a full-length heavy chain or fragments thereof. For example, a full-length heavy chain for human IgG1 has a molecular weight of around 50,000 Daltons and includes a variable region domain (abbreviated herein as V_{H}), and three constant region domains (abbreviated herein as C_{H}1, C_{H}2, and C_{H}3). The term “V_{H} fragment” means a fragment of the heavy chain of a monoclonal antibody that includes all or part of the heavy chain variable region, including CDRs. A V_{H} fragment can further include heavy chain constant region sequences. The number of heavy chain constant region domains will depend on the isotype. The isotype of an antibody can be IgM, IgD, IgG, IgA, or IgE and is defined by the heavy chains present of which there are five classifications: mu (μ), delta (d), gamma (γ), alpha (α), or epsilon (ε) chains, respectively. Human IgG has several subtypes, including, IgG1, IgG2, IgG3, and IgG4.

A. Types of Antibodies

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one immunoglobulin unit. Monomers are the basic functional units of antibodies. The term “dimer” means an antibody containing two immunoglobulin units attached to one another via constant domains of the antibody heavy chains (the Fc, or fragment crystallizable, region). The complex may be stabilized by a joining (J) chain protein. The term “multimer” means an antibody containing more than two immunoglobulin units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificity or they may be bispecific, meaning the two antigen-binding sites have different antigen specificities.

Bispecific antibodies are a class of antibodies that have paratopes (i.e., antigen-binding sites) for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies may be sourced and modified from cartilaginous fish and camelids. Nanobodies can be joined together by a linker using techniques typical to a person skilled in the art, such methods for selection and joining of nanobodies are described in PCT Publication No. WO2015044386A1, No. WO2010037838A2, and Bever et al., Anal Chem. 86(15):7875-7882(2014), each of which are specifically incorporated herein by reference in their entirety.

Bispecific antibodies can be constructed as: a whole IgG, Fab′2, Fab′PEG, a diabody, or alternatively as a single chain variable fragment (scFv). Diabodies and scFvs can be constructed without an Fc region, using only variable domains. Bispecific antibodies may be produced by a variety of methods including, but not limited to, fusion of hybridomas or linking of Fab′ fragments. See, e.g., Songsivilai and Lachmann, Clin. Exp. Immunol. 79(3):315-321 (1990); Kostelny et al., J. Immunol. 148(5):1547-1553 (1992), each of which are specifically incorporated by reference in their entirety.

In certain aspects, the antigen-binding domain may be multispecific or heterospecific by multimerizing with V_{H} and V_{L} region pairs that bind a different antigen. Accordingly, aspects may include, but are not limited to, bispecific, trispecific, tetraspecific, and other multispecific antibodies or antigen-binding fragments thereof that are directed to epitopes and to other targets, such as Fc receptors on effector cells. The antibody may bind to, or interact with, (a) a cell surface antigen, (b) an Fc receptor on the surface of an effector cell, or (c) at least one other component.

In some embodiments, multispecific antibodies can be used and directly linked via a short flexible polypeptide chain, using routine methods known in the art. One such example is diabodies that are bivalent, bispecific antibodies in which the V_{H} and V_{L} domains are expressed on a single polypeptide chain, and utilize a linker that is too short to allow for pairing between domains on the same chain, thereby forcing the domains to pair with complementary domains of another chain creating two antigen-binding sites. The linker functionality is applicable for embodiments of triabodies, tetrabodies, and higher order antibody multimers (see, e.g., Hollinger et al., Proc. Natl. Acad. Sci., USA, 90(14):6444-6448 (1993); Polijak et al., Structure, 2(12):1121-1123 (1994); and Todorovska et al., J. Immunol. Methods, 248(1-2):47-66 (2001), each of which is incorporated herein by reference in their entirety).

The part of the Fv fragment of an antibody molecule that binds with high specificity to the epitope of the antigen is referred to herein as the “paratope.” The paratope consists of the amino acid residues that contact the epitope of an antigen to facilitate antigen recognition. Each of the two Fv fragments of an antibody is composed of the two variable domains, V_{H} and V_{L}, in dimerized configuration. The primary structure of each of the variable domains includes three hypervariable loops separated by, and flanked by, framework regions (FRs). The hypervariable loops are the regions of highest primary sequences variability among the antibody molecules from any mammal. The term hypervariable loop is sometimes used interchangeably with the term “complementarity determining region (CDR).” The length of the hypervariable loops (or CDRs) varies between antibody molecules. The FRs of all antibody molecules from a given mammal have high primary sequence similarity/consensus. The consensus of FRs from different antibodies—typically from the same species—can be used by one skilled in the art to identify both the FRs and the hypervariable loops (or CDRs) which are interspersed among the FRs. The hypervariable loops are given identifying names which distinguish their position within the polypeptide, and on which domain they occur. CDRs in the V_{L} domain are identified as L1 (also CDR-L1), L2 (also CDR-L2), and L3 (also CDR-L3), with L1 occurring at the most distal end with respect to the C_{L} domain and L3 occurring closest to the C_{L} domain. The CDRs may also be given the names CDR1, CDR2, and CDR3. The L3 (CDR3) is generally the region of highest variability in the V_{L} domain among all antibody molecules produced by a given organism. The CDRs are regions of the polypeptide chain arranged linearly in the primary structure and separated from each other by FRs. The amino terminal (N-terminal) end of the V_{L} chain is named FR1. The region identified as FR2 occurs between L1 and L2 hypervariable loops. FR3 occurs between L2 and L3 hypervariable loops, and the FR4 region is closest to the C_{L} domain. This structure and nomenclature are repeated for the V_{H} chain, which includes three CDRs identified as H1 (also CDR-H1), H2 (also CDR-H2), and H3 (also CDR-H3). The H3 (CDR-H3) is generally the region of highest variability in the antibody molecules produced by a given organism. The majority of amino acid residues in the variable domains, or Fv fragments (V_{H} and V_{L}), are part of the FRs (approximately 85%).

Several methods have been developed and can be used by one skilled in the art to identify the amino acids that constitute each of these regions. This can be done using any of a number of multiple sequence alignment methods and algorithms, which identify the conserved amino acid residues that make up the FRs, therefore identifying the CDRs that may vary in length but are located between FRs. Three commonly used numberings have been developed for identification of the CDRs of antibodies: Kabat (as described in Wu and Kabat, J. Exp. Med., 132(2): 211-250 (1970)); Chothia (as described in Chothia et al., Nature, 342(6252): 877-883 (1989)); and IMGT (as described in Lefranc et al., Dev. Comp. Immunol., 27(1): 55-77 (2003)). These methods each include unique numbering systems for the identification of the amino acid residues that constitute the variable regions. In most antibody molecules, the amino acid residues that actually contact the epitope of the antigen occur in the CDRs, although in some cases, residues within the FRs contribute to antigen-binding. Depending on the type and size of the antigen, different CDR residues may contact the antigen. See Almagro, J. Mol. Recognit., 17(2):132-43 (2004), incorporated herein by reference.

One skilled in the art can use any of several methods to determine the paratope of an antibody. These methods include:

1) Computational predictions of the tertiary structure of the antibody/epitope binding interactions based on the chemical nature of the amino acid sequence of the antibody variable region and composition of the epitope.

2) Hydrogen-deuterium exchange and mass spectroscopy.

3) Polypeptide fragmentation and peptide mapping approaches in which one generates multiple overlapping peptide fragments from the full length of the polypeptide and evaluates the binding affinity of these peptides for the epitope.

4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific V_{H} and V_{L} domains as appropriate.

5) X-ray crystallography.

6) Alanine scanning mutagenesis.

In certain aspects, affinity matured antibodies are enhanced with one or more modifications in one or more CDRs thereof (and/or one or more FRs thereof) that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Biotechnology, 10(7):779-783 (1992) describes affinity maturation by V_{H} and V_{L} domain shuffling, random mutagenesis of CDR and/or FRs employed in phage display is described by Rajpal et al., Proc. Natl. Acad. Si. USA, 102(24): 8466-8471 (2005) and Thie et al., Methods Mol Biol., 525:309-322 (2009) in conjugation with computation methods as demonstrated in Tiller et al., Front. Immunol., 8: Article 986 (2017), each of which references are incorporated herein by reference in their entirety.

Chimeric immunoglobulins are the products of fused genes derived from different species; “humanized” antibodies generally have the FRs from human immunoglobulins and one or more CDRs are from a non-human source (e.g., murine).

In some embodiments, minimizing the antibody polypeptide sequence from the non-human species optimizes chimeric antibody function and reduces immunogenicity. Specific amino acid residues of the non-human antibody are modified to be homologous to corresponding residues in a human antibody. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. In some instances, corresponding non-human (e.g., murine) residues replace FR amino acid residues of the human immunoglobulin. Replacement of human FR residues with non-human FR residues may serve to improve and/or restore antigen-binding. Furthermore, humanized antibodies may comprise residues that are not found in the recipient antibody or in the donor antibody to further refine performance. The humanized antibody may also comprise at least a portion of an immunoglobulin constant region, typically that of a human immunoglobulin.

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

A monoclonal antibody or “mAb” refers to an antibody obtained from a population of homogeneous antibodies from an exclusive parental cell, e.g., the population is identical except for naturally occurring mutations that may be present in minor amounts. Each monoclonal antibody is directed against a single antigenic determinant (epitope).

B. Functional Antibody Fragments and Antigen-Binding Fragments

1. Antigen-Binding Fragments

Certain aspects relate to antibody fragments, such as antibody fragments that bind to antigen. The term functional antibody fragment includes antigen-binding fragments of an antibody that retain the ability to specifically bind to an antigen. These fragments are constituted of various arrangements of the variable region heavy chain (V_{H}) and/or light chain (V_{L}); and in some embodiments, include constant region heavy chain 1 (C_{H}1) and light chain (C_{L}). In some embodiments, they lack the Fc region constituted of heavy chain 2 (C_{H}2) and 3 (C_{H}3) domains. Embodiments of antigen-binding fragments and the modifications thereof may include: (i) the Fab fragment type constituted with the V_{L}, V_{H}, C_{L}, and C_{H}1 domains; (ii) the Fd fragment type constituted with the V_{H} and C_{H}1 domains; (iii) the Fv fragment type constituted with the V_{H} and V_{L} domains; (iv) the single domain fragment type, dAb, (Holt et al., Trends Biotechnol., 21(11):484-490 (2003)) constituted with a single V_{H} or V_{L} domain; (v) isolated CDRs. Such terms are described, for example, in Harlow and Lane (Eds.), Antibodies: A Laboratory Manual, Cold Spring Harbor Laboratory, New York, NY, USA (1988); Molecular Biology and Biotechnology: A Comprehensive Desk Reference, Meyers (Ed.), Wiley-VCH Publisher, Inc., New York, NY, USA (1995); Huston et al., Cell Biophys., 22(1-3):189-224 (1993); and Pluckthun and Skerra, Methods Enzymol., 178:497-515 (1989), each of which are incorporated by reference in their entirety.

Antigen-binding fragments also include fragments of an antibody that retain exactly, at least, or at most 1, 2, or 3 CDRs from a light chain variable region. Fusions of CDR-containing sequences to an Fc region (or a C_{H}2 or C_{H}3 region thereof) are included within the scope of this definition including, for example, scFv fused, directly or indirectly, to an Fc region are included herein.

The term Fab fragment means a monovalent antigen-binding fragment of an antibody containing the variable (V_{L} and V_{H}) and the constant (C_{L} and C_{H}1) domains. The term Fab′ fragment means a monovalent antigen-binding fragment of a monoclonal antibody that is larger than a Fab fragment. For example, a Fab′ fragment includes the V_{L}, V_{H}, C_{L} and C_{H}1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two V_{H} and V_{L} domains and can further include all or part of the two C_{L} and C_{H}1 domains.

The term Fd fragment means a fragment of the heavy chain of a monoclonal antibody, which includes all or part of the V_{H}, including the CDRs. An Fd fragment can further include C_{H}1 region sequences.

The term Fv fragment means a monovalent antigen-binding fragment of a monoclonal antibody, including all or part of the V_{L} and V_{H}, and absent of the C_{L} and C_{H}1 domains. The V_{L} and V_{H} include, for example, the CDRs. Single-chain antibodies (sFv or scFv) are Fv molecules in which the V_{L} and V_{H} regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)₂ means bivalent or bispecific sFv polypeptide chains that include oligomerization domains at their C-termini, separated from the sFv by a hinge region. The oligomerization domain comprises self-associating α-helices, e.g., leucine zippers, which can be further stabilized by additional disulfide bonds. (scFv)₂ fragments are also known as “miniantibodies” or “minibodies.”

A single domain antibody is an antigen-binding fragment containing only a V_{H} or the V_{L} domain. In some instances, two or more V_{H} regions are covalently joined with a peptide linker to create a bivalent domain antibody. The two V_{H} regions of a bivalent domain antibody may target the same or different antigens.

2. Fragment Crystallizable Region, Fc

In some cases, including for IgG, IgD, and IgA antibodies, an Fc region contains two heavy chain fragments comprising the C_{H}2 and C_{H}3 domains of an antibody. The two heavy chain fragments are held together by two or more disulfide bonds and by hydrophobic interactions of the C_{H}3 domains. The term “Fc polypeptide” as used herein includes native and mutein forms of polypeptides derived from the Fc region of an antibody. Truncated forms of such polypeptides containing a hinge region that promotes dimerization are included.

C. Polypeptides with Antibody CDRs & Scaffolding Domains that Display the CDRs

Antigen-binding peptide scaffolds, such as CDRs, are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify (Skerra, J. Mol. Recognit., 13(4):167-187 (2000)).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibitors of neuronal nitric oxide synthase (PIN) may also be used.

D. Antibody Binding

The term “selective binding agent”, “antigen-binding agent”, or “antigen-binding protein” refers to a molecule that binds to an antigen. Non-limiting examples include antibodies, antigen-binding fragments, scFv, Fab, Fab′, F(ab′)₂, single chain antibodies, aptamers, peptides, peptide fragments, and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (K_{D} or Kd), using the equation K_{D}=koff/kon=[A][B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the K_{D} are mol/L (molarity, or M), or concentration. The Ka of an antibody is the inverse of the K_{D}, and is determined by the equation Ka=1/K_{D}. Examples of some experimental methods that can be used to determine the K_{D} value are: enzyme-linked immunosorbent assays (ELISA), isothermal titration calorimetry (ITC), fluorescence anisotropy, surface plasmon resonance (SPR), and affinity capillary electrophoresis (ACE).

Antibodies deemed useful in certain embodiments may have an equilibrium dissociation constant of about, at least about or at most about 10⁻⁶, 10⁻⁷, 10⁻⁸, 10⁻⁹, 10⁻¹⁰ M, 10⁻¹¹ M, 10⁻¹² M, or any range derivable therein.

2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B- and/or T-cell receptors respond or recognize. Polypeptide epitopes can be formed from both contiguous amino acids and noncontiguous amino acids juxtaposed by tertiary folding of a polypeptide. In some embodiments, an epitope includes at least 3, for example 5-10 amino acids, in a unique spatial conformation.

Epitope specificity of an antibody can be determined in a variety of ways. One approach, for example, involves testing a collection of overlapping peptides of about 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N- and C-terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

3. Modification of Antibody Antigen-Binding Domains

It is understood that the antibodies of the present disclosure may be modified, such that they are substantially identical to the antibody polypeptide sequences, or fragments thereof, and still bind the epitopes of the present disclosure. Polypeptide sequences are “substantially identical” when optimally aligned using such programs as Clustal Omega, IGBLAST, GAP, or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present disclosure, providing that the variations in the amino acid sequence maintain at least 75%, more preferably at least 80%, at least 90%, at least 95%, at least 96%, at least 97%, at least 98% and most preferably at least 99% sequence identity. In some embodiments, conservative amino acid replacements are contemplated.

Conservative replacements (also “conservative substitutions” or “conservative amino acid substitutions”) are those that take place within a family of amino acids that possess similar biochemical properties, including charge, hydrophobicity, and size. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). Thus, a conservative replacement may comprise replacement of an amino acid in one family for an amino acid in the same family (e.g., replacement of a lysine with an arginine, replacement of an aspartate for a glutamate, etc.). Alternatively or in addition, amino acid similarity may be determined using a Blocks Substitution Matrix (BLOSUM), such as BLOSUM62 (Henikoff and Henikoff, Proc. Natl. Acad. Sci. USA, 89(22): 10915-9 (1992)). In this case, a conservative replacement may be a substitution of amino acids having a non-negative value on a BLOSUM62 matrix. Whether an amino acid change results in a functional peptide can readily be determined by assaying the specific activity of the polypeptide derivative. Standard ELISA, SPR, or other antibody-binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Certain preferred N- and C-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art (Dill and MacCallum, Science, 338(6110):1042-1046 (2012)). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

It is also contemplated that the antigen-binding domain may be multi-specific or multivalent by multimerizing the antigen-binding domain with V_{H} and V_{L} region pairs that bind either the same antigen (multi-valent) or a different antigen (multi-specific).

E. Enzymatic and Chemical Modification of Antibodies

In some aspects, also contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861, incorporated herein by reference). In certain embodiments, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other embodiments, one or more new N-linked glycosylation sites are created.

Additional antibody variants include cysteine variants, wherein one or more cysteine residues in the parent or native amino acid sequence are deleted from or substituted with another amino acid (e.g., serine). Cysteine variants are useful, inter aha, when antibodies must be refolded into a biologically active conformation. Cysteine variants may have fewer cysteine residues than the native antibody and typically have an even number to minimize interactions resulting from unpaired cysteines.

In some aspects, the polypeptides can be PEGgylated to increase the biological half-life by reacting the polypeptide with polyethylene glycol (PEG) or a reactive ester or aldehyde derivative of PEG, under conditions in which one or more PEG groups become attached to the polypeptide. Polypeptide PEGylation may be carried out by an acylation reaction or an alkylation reaction with a reactive PEG molecule (or an analogous reactive water-soluble polymer). Methods for PEGylating proteins are known in the art and can be applied to the polypeptides of the disclosure to obtain PEGylated derivatives of antibodies. See, e.g., EP 0154316 and EP 0401384, incorporated herein by reference. In some aspects, the antibody is conjugated or otherwise linked to transthyretin (TTR) or a TTR variant. The TTR or TTR variant can be chemically modified with, for example, a chemical selected from the group consisting of dextran, poly(n-vinylpyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

1. Conjugation

Derivatives of the antibodies and antigen-binding fragments that are described herein are also provided. The derivatized antibody or fragment thereof may comprise any molecule or substance that imparts a desired property to the antibody or fragment. The derivatized antibody can comprise, for example, a detectable (or labeling) moiety (e.g., a radioactive, colorimetric, antigenic, or enzymatic molecule, or a detectable bead), a molecule that binds to another molecule (e.g., biotin/streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses). In some embodiments, an antibody or fragment thereof is covalently attached to a molecule or substance, such as a labeling moiety or a therapeutic moiety. In some embodiments, an antibody or fragment thereof is non-covalently attached to a molecule or substance, such as a labeling moiety or a therapeutic moiety.

Optionally, an antibody or an antigen-binding fragment can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0486525. In some aspects, the polypeptides may be conjugated to a diagnostic agent and used diagnostically, for example, to monitor the development or progression of a disease and determine the efficacy of a given treatment regimen. In some aspects, the polypeptides may also be conjugated to a therapeutic agent to provide a therapy in combination with the therapeutic effect of the polypeptide. Example conjugated molecules are a chemotherapeutic drug, a nucleic acid (e.g. an antisense oligonucleotide, a siRNA or a CRISPR-based gene therapy, etc.), a protein (e.g. a toxin, an enzyme, etc.), a viral vector, or a nanodrug. Additional suitable conjugated molecules include ribonuclease (RNase), DNase I, an antisense oligonucleotide, an inhibitory RNA molecule such as a siRNA molecule, an immunostimulatory nucleic acid, aptamers, ribozymes, triplex forming molecules, and external guide sequences (e.g., guide RNAs). The functional nucleic acid molecules may act as effectors, inhibitors, modulators, and stimulators of a specific activity possessed by a target molecule, or the functional nucleic acid molecules may possess a de novo activity independent of any other molecules.

In some aspects, disclosed are antibodies and antibody-like molecules that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

a. Conjugate Types

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., avidin and streptavidin) and the like. Particular examples of labels are, but not limited to, fluorescein, fluorescein isothiocyanate (FITC), rhodamine, dansyl, umbelliferone, dimethyl acridinium ester (DMAE), Texas red, phycoerythrin (PE), and luminol. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase (AP), horseradish peroxidase (HRP), α- or β-galactosidase, and glucose oxidase. Preferred secondary binding ligands are avidin and streptavidin compounds that are capable of binding biotin with high affinity. The uses of such labels is well known to those of skill in the art and are described, for example, in U.S. Pat. Nos. 3,817,837; 3,850,752; 3,939,350; 3,996,345; 4,277,437; 4,275,149; and 4,366,241, each incorporated herein by reference. Molecules containing azido groups may also be used to form covalent bonds to proteins through reactive nitrene intermediates that are generated by low intensity ultraviolet light.

In some aspects, contemplated are immunoconjugates comprising an antibody or antigen-binding fragment thereof conjugated (e.g., covalently attached) to a cytotoxic agent such as a chemotherapeutic agent, a drug, a nucleic acid (e.g., antisense oligonucleotide, siRNA, etc.) a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing the targeted cell surface antigen. The antibody and the agent may be associated through non-covalent interactions such as through electrostatic forces, or by covalent bonds. Various linkers, known in the art, can be employed in order to form the immunoconjugate. Additionally, the immunoconjugate can be provided in the form of a genetic fusion protein. In one aspect, an antibody may be conjugated to various therapeutic substances in order to target the cell surface antigen. Examples of conjugated agents include, but are not limited to, metal chelate complexes, drugs, toxins and other effector molecules, such as cytokines, lymphokines, chemokines, immunomodulators, radiosensitizers, asparaginase, carboranes, and radioactive halogens.

In antibody drug conjugates (ADCs), an antibody is conjugated to one or more drug moieties (e.g., small molecule drugs such as chemotherapeutics) through a linker. The ADCs may be prepared by several routes, employing organic chemistry reactions, conditions, and reagents known to those skilled in the art, including: (1) reaction of a nucleophilic group of an antibody with a bivalent linker reagent, to form antibody-L, via a covalent bond, followed by reaction with a drug moiety D; and (2) reaction of a nucleophilic group of a drug moiety with a bivalent linker reagent, to form drug-linker (D-L), via a covalent bond, followed by reaction with the nucleophilic group of an antibody. ADCs may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

In certain aspects, ADCs include covalent or aggregative conjugates of antibodies, or antigen-binding fragments thereof, with other proteins or peptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG® (Sigma-Aldrich, St. Louis, MO., USA) peptide as described in Hopp et al., Bio/Technology, 6:1204-1210 (1988) and U.S. Pat. No. 5,011,912.

Also contemplated herein are activatable immunoconjugates comprising an antibody or antigen binding fragment thereof conjugated to a therapeutic agent, and further comprising a masking moiety, wherein the masking moiety reduces the ability of the antibody or antigen-binding fragment thereof to bind to an antigen (e.g., TfR1). A masking moiety may be conjugated to an antigen-binding protein of the disclosure via a linker having a protease cleavage site, where the masking moiety is removed via protease activity in a tumor microenvironment, thereby activating the antigen-binding protein. Certain non-limiting examples of activatable antibodies, antibody fragments, and immunoconjugates (e.g., ADCs) are described in U.S. Pat. No. 10,179,817, incorporated herein by reference. In some embodiments, disclosed is an activatable anti-CD138 antibody or antigen-binding fragment thereof. In some embodiments, disclosed is an activatable anti-CD38 antibody or antigen-binding fragment thereof. In some embodiments, disclosed is an activatable anti-TfR1 antibody or antigen-binding fragment thereof.

b. Conjugation Methodology

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis(p-diazoniumbenzoyl)-ethylenediamine), diisocyanates (such as toluene 2,6-diisocyanate), and bis-active fluorine compounds (such as 1,5-difluoro-2,4-dinitrobenzene). In some aspects, derivatization of immunoglobulins by selectively introducing sulfhydryl groups in the Fc region of an immunoglobulin, using reaction conditions that do not alter the antibody combining site, are contemplated. Antibody conjugates produced according to this methodology are disclosed to exhibit improved longevity, specificity, and sensitivity (U.S. Pat. No. 5,196,066, incorporated herein by reference). Site-specific attachment of effector or reporter molecules, wherein the reporter or effector molecule is conjugated to a carbohydrate residue in the Fc region has also been disclosed in the literature (O'Shannessy et al., J. Immunol. Methods, 99(2):153-161 (1987).

F. Proteins

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOs:1-32.

In some embodiments, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOs:1-32.

In some embodiments, the protein, polypeptide, or nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOs:1-32.

In some embodiments, the polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOs:1-32 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS:1-32.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOS:1-32 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOS:1-32.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

1. Tumor Antigens

Certain aspects of the present disclosure are directed to antibodies, including engineered antibodies, configured to bind to a tumor antigen. A tumor antigen includes any protein, polypeptide, peptide, or other component of a tumor or cancer cell capable of being targeted by a targeting molecule such as an antibody. In some embodiments, a tumor antigen is a protein that is overexpressed in a cancer cell relative to heathy cells of the same tissue type. In some embodiments, a tumor antigen is a protein that is expressed in a cancer cell that is not expressed in a healthy cell of the same tissue type. Examples of tumor antigens disclosed herein include CD138, CD38, and transferrin receptor 1 (TfR1; also “transferrin receptor protein 1”).

CD138, also known as syndecan-1, is a glycoprotein receptor involved in cell adhesion, cell-matrix interaction, cellular proliferation, and angiogenesis¹⁹. In cancer, CD138 is present on a number of epithelial and hematological malignancies but it is best known as a marker of multiple myeloma (MM) involved in carcinogenesis, cell proliferation, angiogenesis, and metastasis¹⁹⁻²⁴, making it a meaningful target for antibody-based diagnostics and therapeutics in MM²⁵⁻²⁸. Importantly, CD138 is also expressed by putative myeloma stem cells, meaning that this relevant malignant cell population may be eliminated by therapies targeting CD138²⁷. CD138 is also expressed on other cancers including non-Hodgkin lymphoma (NHL) and epithelial tumors such as prostate, breast (including triple negative breast cancer (TNBC)), colorectum, and kidney cancers, and has also been associated with poor prognosis^{21,29-33}. In some embodiments, disclosed are engineered antibodies configured to bind CD138 (also “CD138-targeted antibodies”). CD138-targeted antibodies may comprise one or more regions (e.g., CDRs, V_{H} domain, C_{L} domain) from a CD138-binding antibody. Engineered CD138-targeted antibodies may further comprise one or more regions from an IgE antibody. Examples of CD138-binding antibodies include B-B4, BC/B-B4, B-B2, DL-101, 1D4, M115, 1.BB.210, 2Q1484, 5F7, 104-9, and 281-2. Example CD138-binding antibodies are described in U.S. Pat. No. 9,221,914, incorporated herein by reference in its entirety. Embodiments of the disclosure are directed to methods of use of CD138-targeted antibodies for treatment of a subject having a cancer expressing CD138.

CD38, also known as ADP-ribosyl cyclase/cyclic ADP-ribose hydrolase, is a glycoprotein expressed at high levels on a large number of hematopoietic malignancies compared to normal cells^{34,35}. High expression of CD38 has been associated with acute lymphoblastic leukemia (ALL)³⁶⁻³⁷, acute myeloid leukemia (AML)^{36,37}, aggressive natural killer (NK) cell leukemia (ANKL)³⁸, NK/T-cell lymphoma³⁹, and mantle cell lymphoma (MCL)⁴⁰. CD38 shows especially high and uniform expression levels in MM cells^{24,34,35,41} making it an attractive target for MM therapy^{34,35,41-44}. In some embodiments, disclosed are engineered antibodies configured to bind CD38 (also “CD38-targeted antibodies”). CD38-targeted antibodies may comprise one or more regions (e.g., CDRS, V_{H} region, C_{L} region) from a CD38-binding antibody. Engineered CD38-targeted antibodies may further comprise one or more regions from an IgE antibody. Examples of CD38-binding antibodies include isatuximab (Sarclisa®), felzartamab (MOR202), and daratumumab (Darzalex®). Example CD38-binding antibodies are described in US Patent Application Publication US 2017/0174780, incorporated herein by reference in its entirety. Embodiments of the disclosure are directed to methods of use of CD38-targeted antibodies for treatment of a subject having a cancer expressing CD38.

Transferrin receptor protein 1 (TfR1), also known as CD71, is expressed at high levels on malignancies^{17,18,45-83}. TfR1 has been identified as a universal cancer marker⁶³. Increased expression of TfR1 correlates with advanced stage and/or poorer prognosis in several malignancies, including solid cancers such as esophageal squamous cell carcinoma⁶⁴, breast cancer^{65,66}, ovarian cancer⁶⁷, lung cancer⁶⁸, cervical cancer⁶⁹, bladder cancer⁷⁰, osteosarcoma⁷¹, pancreatic cancers⁷², cholangiocarcinoma⁷³, renal cell carcinoma⁷⁴, hepatocellular carcinoma^{17,18}, adrenal cortical carcinoma⁷⁵, and malignancies of the nervous system such as glioblastomas^{51,76} as well as hematopoietic malignancies such as ALL^{77,79}, chronic lymphocytic leukemia (CLL)⁷⁸, and non-Hodgkin lymphoma (NHL)⁷⁸⁻⁸⁰. Individuals infected with the human immunodeficiency virus (HIV) develop more aggressive NHL, which express even higher TfR1 levels compared to NHL cells from non-infected individuals^{81,82}. In some embodiments, disclosed are engineered antibodies configured to bind TfR1 (also “TfR1-targeted antibodies”). TfR1-targeted antibodies may comprise one or more regions (e.g., CDRs, V_{H} domain, C_{L} domain) from a TfR1-binding antibody. Engineered TfR1-targeted antibodies may further comprise one or more regions from an IgE antibody. Examples of TfR1-binding antibodies include 128.1, ch128.1, 7579, E2.3, A27.15, B3/25, 43/31, D65.30, A24, RBC4, 42/6, D2C, JST-TFR09, and H7¹¹⁹⁻¹²⁸. Certain example TfR1-binding antibodies are described in U.S. Pat. Nos. 8,734,799 and 6,329,508, incorporated herein by reference in their entirety. Embodiments of the disclosure are directed to methods of use of TfR1-targeted antibodies for treatment of a subject having a cancer expressing TfR1.

2. Sequences

Amino acid sequences from certain CD38-targeted engineered antibodies of the present disclosure are provided in SEQ ID NOs: 11-18 and SEQ ID NOs: 9-10 as follows in

TABLE 1

| | SEQ ID | |
|---|---|---|
| Polypeptide | NO: | Sequence |
| Daratumumab (anti- | 11 | SFAMS |
| CD38) V_{H} CDR1 | | |
| Daratumumab (anti- | 12 | AISGSGGGTYYADSVKG |
| CD38) V_{H} CDR2 | | |
| Daratumumab (anti- | 13 | DKILWFGEPVFDY |
| CD38) V_{H} CDR3 | | |
| Daratumumab (anti- | 14 | RASQSVSSYLA |
| CD38) V_{L} CDR1 | | |
| Daratumumab (anti- | 15 | DASNRAT |
| CD38) V_{L} CDR2 | | |
| Daratumumab (anti- | 16 | QQRSNWPPTF |
| CD38) V_{L} CDR3 | | |
| Daratumumab (anti- | 17 | EVQLLESGGGLVQPGGSLRLSCAVSGFTFNSFAM |
| CD38) V_{H} | | SWVRQAPGKGLEWVSAISGSGGGTYYADSVKG |
| | | RFTISRDNSKNTLYLQMNSLRAEDTAVYFCAKD |
| | | KILWFGEPVFDYWGQGTLVTVSS |
| Daratumumab (anti- | 18 | EIVLTQSPATLSLSPGERATLSCRASQSVSSYLAW |
| CD38) V_{L} | | YQQKPGQAPRLLIYDASNRATGIPARFSGSGSGT |
| | | DFTLTISSLEPEDFAVYYCQQRSNWPPTFGQGTK |
| | | VEIK |
| Heavy Chain | 9 | ASTQSPSVFPLTRCCKNIPSNATSVTLGCLATGYF |
| Epsilon C_{H} | | PEPVMVTWDTGSLNGTTMTLPATTLTLSGHYATI |
| | | SLLTVSGAWAKQMFTCRVAHTPSSTDWVDNKT |
| | | FSVCSRDFTPPTVKILQSSCDGGGHFPPTIQLLCL |
| | | VSGYTPGTINITWLEDGQVMDVDLSTASTTQEGE |
| | | LASTQSELTLSQKHWLSDRTYTCQVTYQGHTFE |
| | | DSTKKCADSNPRGVSAYLSRPSPFDLFIRKSPTIT |
| | | CLVVDLAPSKGTVNLTWSRASGKPVNHSTRKEE |
| | | KQRNGTLTVTSTLPVGTRDWIEGETYQCRVTHP |
| | | HLPRALMRSTTKTSGPRAAPEVYAFATPEWPGSR |
| | | DKRTLACLIQNEMPEDISVQWLHNEVQLPDARH |
| | | STTQPRKTKGSGFFVFSRLEVTRAEWEQKDEFIC |
| | | RAVHEAASPSQTVQRAVSVNPGK |
| Light Chain Kappa | 10 | RTVAAPSVFIFPPSDEQLKSGTASVVCLLNNFYPR |
| C_{L} | | EAKVQWKVDNALQSGNSQESVTEQDSKDSTYSL |
| | | SSTLTLSKADYEKHKVYACEVTHQGLSSPVTKSF |
| | | NRGEC |
| CD38-Targeted | 29 | EVQLLESGGGLVQPGGSLRLSCAVSGFTFNSFAM |
| Antibody Heavy | | SWVRQAPGKGLEWVSAISGSGGGTYYADSVKG |
| Chain | | RFTISRDNSKNTLYLQMNSLRAEDTAVYFCAKD |
| | | KILWFGEPVFDYWGQGTLVTVSSASTQSPSVFPL |
| | | TRCCKNIPSNATSVTLGCLATGYFPEPVMVTWDT |
| | | GSLNGTTMTLPATTLTLSGHYATISLLTVSGAWA |
| | | KQMFTCRVAHTPSSTDWVDNKTFSVCSRDFTPP |
| | | TVKILQSSCDGGGHFPPTIQLLCLVSGYTPGTINIT |
| | | WLEDGQVMDVDLSTASTTQEGELASTQSELTLS |
| | | QKHWLSDRTYTCQVTYQGHTFEDSTKKCADSNP |
| | | RGVSAYLSRPSPFDLFIRKSPTITCLVVDLAPSKG |
| | | TVNLTWSRASGKPVNHSTRKEEKQRNGTLTVTS |
| | | TLPVGTRDWIEGETYQCRVTHPHLPRALMRSTT |
| | | KTSGPRAAPEVYAFATPEWPGSRDKRTLACLIQN |
| | | FMPEDISVQWLHNEVQLPDARHSTTQPRKTKGS |
| | | GFFVFSRLEVTRAEWEQKDEFICRAVHEAASPSQ |
| | | TVQRAVSVNPGK |
| CD38-Targeted | 30 | EIVLTQSPATLSLSPGERATLSCRASQSVSSYLAW |
| Antibody Light | | YQQKPGQAPRLLIYDASNRATGIPARFSGSGSGT |
| Chain | | DFTLTISSLEPEDFAVYYCQQRSNWPPTFGQGTK |
| | | VEIKRTVAAPSVFIFPPSDEQLKSGTASVVCLLNN |
| | | FYPREAKVQWKVDNALQSGNSQESVTEQDSKDS |
| | | TYSLSSTLTLSKADYEKHKVYACEVTHQGLSSPV |
| | | TKSFNRGEC |

Amino acid sequences from certain CD138-targeted engineered antibodies of the present disclosure are provided in SEQ ID NOs: 1-8 and SEQ ID NOs: 9-10 as follows in Table 1:

| | SEQ ID | |
|---|---|---|
| Polypeptide | NO: | Sequence |
| B-B4 (anti-CD138) | 1 | NYWIE |
| V_{H} CDR1 | | |
| B-B4 (anti-CD138) | 2 | ILPGTGRTIYNEKFKGKA |
| V_{H} CDR2 | | |
| B-B4 (anti-CD138) | 3 | RDYYGNFYYAMDY |
| V_{H} CDR3 | | |
| B-B4 (anti-CD138) | 4 | ASQGINNYLN |
| V_{L} CDR1 | | |
| B-B4 (anti-CD138) | 5 | TSTLQS |
| V_{L} CDR2 | | |
| B-B4 (anti-CD138) | 6 | QQYSKLPRTF |
| V_{L} CDR3 | | |
| B-B4 (anti-CD138) | 7 | QVQLQQSGSELMMPGASVKISCKATGYTFSNYW |
| V_{H} | | IEWVKQRPGHGLEWIGEILPGTGRTIYNEKFKGK |
| | | ATFTADISSNTVQMQLSSLTSEDSAVYYCARRDY |
| | | YGNFYYAMDYWGQGTSVTVSS |
| B-B4 (anti-CD138) | 8 | DIQMTQSTSSLSASLGDRVTISCSASQGINNYLN |
| V_{L} | | WYQQKPDGTVELLIYYTSTLQSGVPSRFSGSGSG |
| | | TDYSLTISNLEPEDIGTYYCQQYSKLPRTFGGGTK |
| | | LEIK |
| Heavy Chain | 9 | ASTQSPSVFPLTRCCKNIPSNATSVTLGCLATGYF |
| Epsilon C_{H} | | PEPVMVTWDTGSLNGTTMTLPATTLTLSGHYATI |
| | | SLLTVSGAWAKQMFTCRVAHTPSSTDWVDNKT |
| | | FSVCSRDFTPPTVKILQSSCDGGGHFPPTIQLLCL |
| | | VSGYTPGTINITWLEDGQVMDVDLSTASTTQEGE |
| | | LASTQSELTLSQKHWLSDRTYTCQVTYQGHTFE |
| | | DSTKKCADSNPRGVSAYLSRPSPFDLFIRKSPTIT |
| | | CLVVDLAPSKGTVNLTWSRASGKPVNHSTRKEE |
| | | KQRNGTLTVTSTLPVGTRDWIEGETYQCRVTHP |
| | | HLPRALMRSTTKTSGPRAAPEVYAFATPEWPGSR |
| | | DKRTLACLIQNEMPEDISVQWLHNEVQLPDARH |
| | | STTQPRKTKGSGFFVFSRLEVTRAEWEQKDEFIC |
| | | RAVHEAASPSQTVQRAVSVNPGK |
| Light Chain Kappa | 10 | RTVAAPSVFIFPPSDEQLKSGTASVVCLLNNFYPR |
| C_{L} | | EAKVQWKVDNALQSGNSQESVTEQDSKDSTYSL |
| | | SSTLTLSKADYEKHKVYACEVTHQGLSSPVTKSF |
| | | NRGEC |
| CD138-Targeted | 27 | QVQLQQSGSELMMPGASVKISCKATGYTFSNYW |
| Antibody Heavy | | IEWVKQRPGHGLEWIGEILPGTGRTIYNEKFKGK |
| Chain | | ATFTADISSNTVQMQLSSLTSEDSAVYYCARRDY |
| | | YGNFYYAMDYWGQGTSVTVSSASTQSPSVFPLT |
| | | RCCKNIPSNATSVTLGCLATGYFPEPVMVTWDT |
| | | GSLNGTTMTLPATTLTLSGHYATISLLTVSGAWA |
| | | KQMFTCRVAHTPSSTDWVDNKTFSVCSRDFTPP |
| | | TVKILQSSCDGGGHFPPTIQLLCLVSGYTPGTINIT |
| | | WLEDGQVMDVDLSTASTTQEGELASTQSELTLS |
| | | QKHWLSDRTYTCQVTYQGHTFEDSTKKCADSNP |
| | | RGVSAYLSRPSPFDLFIRKSPTITCLVVDLAPSKG |
| | | TVNLTWSRASGKPVNHSTRKEEKQRNGTLTVTS |
| | | TLPVGTRDWIEGETYQCRVTHPHLPRALMRSTT |
| | | KTSGPRAAPEVYAFATPEWPGSRDKRTLACLIQN |
| | | FMPEDISVQWLHNEVQLPDARHSTTQPRKTKGS |
| | | GFFVFSRLEVTRAEWEQKDEFICRAVHEAASPSQ |
| | | TVQRAVSVNPGK |
| CD138-Targeted | 28 | DIQMTQSTSSLSASLGDRVTISCSASQGINNYLN |
| Antibody Light | | WYQQKPDGTVELLIYYTSTLQSGVPSRFSGSGSG |
| Chain | | TDYSLTISNLEPEDIGTYYCQQYSKLPRTFGGGTK |
| | | LEIKRTVAAPSVFIFPPSDEQLKSGTASVVCLLNN |
| | | FYPREAKVQWKVDNALQSGNSQESVTEQDSKDS |
| | | TYSLSSTLTLSKADYEKHKVYACEVTHQGLSSPV |
| | | TKSFNRGEC |

Amino acid sequences from certain TfR1-targeted engineered antibodies of the present disclosure are provided in SEQ ID NOs: 19-26 and SEQ ID NOs: 9-10 as follows in Table 1:

| | SEQ ID | |
|---|---|---|
| Polypeptide | NO: | Sequence |
| ch128.1 (anti-TfR1) | 19 | GYSFTGYTMN |
| V_{H} CDR1 | | |
| ch128.1 (anti-TfR1) | 20 | RINPHNGGTDYNQKFKD |
| V_{H} CDR2 | | |
| ch128.1 (anti-TfR1) | 21 | GYYYYSLDY |
| V_{H} CDR3 | | |
| ch128.1 (anti-TfR1) | 22 | SASSSIRYIH |
| V_{L} CDR1 | | |
| ch128.1 (anti-TfR1) | 23 | DTSNLASGVPA |
| V_{L} CDR2 | | |
| ch128.1 (anti-TfR1) | 24 | HQRNSYPW |
| V_{L} CDR3 | | |
| ch128.1 (anti-TfR1) | 25 | EVQLQQSGPELVKPGASMKISCKASGYSFTGYT |
| V_{H} | | MNWVKQSHGENLEWIGRINPHNGGTDYNQKFK |
| | | DKAPLTVDKSSNTAYMELLSLTSGDSAVYYCAR |
| | | GYYYYSLDYWGQGTSVTVSS |
| ch 128.1 (anti-TfR1) | 26 | QIVLTQSPAIMSVSPGEKVTMTCSASSSIRYIHWY |
| V_{L} | | QQRPGTSPKRWIYDTSNLASGVPARFSGSGSGTS |
| | | YSLTISSMEAEDAATYYCHQRNSYPWTFGGGTR |
| | | LEIR |
| Heavy Chain | 9 | ASTQSPSVFPLTRCCKNIPSNATSVTLGCLATGYF |
| Epsilon C_{H} | | PEPVMVTWDTGSLNGTTMTLPATTLTLSGHYATI |
| | | SLLTVSGAWAKQMFTCRVAHTPSSTDWVDNKT |
| | | FSVCSRDFTPPTVKILQSSCDGGGHFPPTIQLLCL |
| | | VSGYTPGTINITWLEDGQVMDVDLSTASTTQEGE |
| | | LASTQSELTLSQKHWLSDRTYTCQVTYQGHTFE |
| | | DSTKKCADSNPRGVSAYLSRPSPFDLFIRKSPTIT |
| | | CLVVDLAPSKGTVNLTWSRASGKPVNHSTRKEE |
| | | KQRNGTLTVTSTLPVGTRDWIEGETYQCRVTHP |
| | | HLPRALMRSTTKTSGPRAAPEVYAFATPEWPGSR |
| | | DKRTLACLIQNFMPEDISVQWLHNEVQLPDARH |
| | | STTQPRKTKGSGFFVFSRLEVTRAEWEQKDEFIC |
| | | RAVHEAASPSQTVQRAVSVNPGK |
| Light Chain Kappa | 10 | RTVAAPSVFIFPPSDEQLKSGTASVVCLLNNFYPR |
| C_{L} | | EAKVQWKVDNALQSGNSQESVTEQDSKDSTYSL |
| | | SSTLTLSKADYEKHKVYACEVTHQGLSSPVTKSF |
| | | NRGEC |
| TfR1-Targeted | 31 | EVQLQQSGPELVKPGASMKISCKASGYSFTGYT |
| Antibody Heavy | | MNWVKQSHGENLEWIGRINPHNGGTDYNQKFK |
| Chain | | DKAPLTVDKSSNTAYMELLSLTSGDSAVYYCAR |
| | | GYYYYSLDYWGQGTSVTVSSASTQSPSVFPLTRC |
| | | CKNIPSNATSVTLGCLATGYFPEPVMVTWDTGSL |
| | | NGTTMTLPATTLTLSGHYATISLLTVSGAWAKQ |
| | | MFTCRVAHTPSSTDWVDNKTFSVCSRDFTPPTV |
| | | KILQSSCDGGGHFPPTIQLLCLVSGYTPGTINITW |
| | | LEDGQVMDVDLSTASTTQEGELASTQSELTLSQK |
| | | HWLSDRTYTCQVTYQGHTFEDSTKKCADSNPRG |
| | | VSAYLSRPSPFDLFIRKSPTITCLVVDLAPSKGTV |
| | | NLTWSRASGKPVNHSTRKEEKQRNGTLTVTSTL |
| | | PVGTRDWIEGETYQCRVTHPHLPRALMRSTTKTS |
| | | GPRAAPEVYAFATPEWPGSRDKRTLACLIQNFMP |
| | | EDISVQWLHNEVQLPDARHSTTQPRKTKGSGFFV |
| | | FSRLEVTRAEWEQKDEFICRAVHEAASPSQTVQR |
| | | AVSVNPGK |
| TfR1-Targeted | 32 | QIVLTQSPAIMSVSPGEKVTMTCSASSSIRYIHWY |
| Antibody Light | | QQRPGTSPKRWIYDTSNLASGVPARFSGSGSGTS |
| Chain | | YSLTISSMEAEDAATYYCHQRNSYPWTFGGGTR |
| | | LEIRRTVAAPSVFIFPPSDEQLKSGTASVVCLLNN |
| | | FYPREAKVQWKVDNALQSGNSQESVTEQDSKDS |
| | | TYSLSSTLTLSKADYEKHKVYACEVTHQGLSSPV |
| | | TKSFNRGEC |

3. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines its functional activity, certain amino acid substitutions can be made in a protein sequence and in its corresponding DNA coding sequence, and nevertheless produce a protein with similar or desirable properties.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90/6, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ nucleic acid sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual amino acid residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative” (also “nonconservative”) In some embodiments, a non-conservative substitution affects a function or activity of the polypeptide. In some embodiments, a non-conservative substitution does not affect a function or activity of the polypeptide. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

4. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8), phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte and Doolittle, J. Mol. Bio., 157(1):105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0), aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

G. Nucleic Acids

In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, polymerase chain reaction (PCR) primers or sequencing primers for identifying, analyzing, mutating, or amplifying a polynucleotide encoding a polypeptide, antisense oligonucleotides for inhibiting expression of a polynucleotide, and complementary sequences of the foregoing described herein. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, 99%, or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, or at least 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000, or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

1. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However, it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449(3):581-594 (2013), incorporated herein by reference. For example, the mutation can quantitatively or qualitatively change the biological activity. Examples of quantitative changes include increasing, reducing, or eliminating the activity. Examples of qualitative changes include altering the antigen specificity of an antibody.

### II. IgE Antibodies

IgE antibodies have several advantages for use in cancer therapeutics, compared to the IgG class that is commonly used for cancer therapy¹⁸. Among these properties are the much higher (two-three orders of magnitude) affinity of IgE for its FcεRs compared to IgG for its FcγRs, allowing a more effective arming of effector cells that would target and eliminate cancer cells^{3-5,9,12}. IgE is a cytophilic antibody with a long half-life on the surface of effector cells³⁻⁷. In addition, the low serum levels of endogenous IgE (generally 100,000 fold lower than IgG) results in less competition for FcR occupancy^{3,4,9,12}. In fact, the loss of specific IgG therapeutics from effector cells due to the high levels of competing serum IgG for host FcγR expressing cells limits ADCC 4. ADCC is a critical mechanism of protection of antibodies against cancer^{85,86}. This explains the need for high doses of therapeutic IgG, which increases the cost of treatment and the probability of adverse effects. Additionally, IgE does not have an inhibitory FcR, while IgG binds to the inhibitory FcγRIIB (CD32B), decreasing ADCC, antibody-dependent cell-mediated phagocytosis (ADCP), and antibody-mediated antigen presentation^{85,87}.

Importantly, the FcεRs are expressed on key effector cells that elicit ADCC, ADCP, and/or antigen presentation such as mast cells, eosinophils, macrophages, DC, and Langerhans cells^{4,9,12} Importantly, several of these cells, such as mast cells and macrophages, naturally infiltrate tumors⁸⁸⁻⁹⁴. When bound to a tumor specific IgE, these cells would be capable of mediating anti-tumor activity through the release of multiple anti-tumor agents as well as phagocytosis (ADCP) in the case of macrophages⁹¹⁻⁹⁹. Thus, targeting IgE in the tumor microenvironment would trigger a localized immediate hypersensitivity (anaphylactic) reaction triggered by mast cell degranulation in the tumor leading to its rapid tumor destruction with decreased chance for tumor escape. Not all cancer cells need to be directly targeted since other malignant cells may be affected by a bystander effect. Macrophages would also kill cancer cells by ADCC and ADCP and, as they are also antigen-presenting cells, would elicit a secondary anti-tumor immune response. Other effector cells, such as basophils and eosinophils, are also expected to contribute to contribute to IgE mediated anti-tumor activity. Importantly, the IgE antibody has a superior capacity, compared to IgG, to trigger antigen presentation in macrophages and dendritic cells through the engagement of FcεRI and FcεRII¹⁰⁰⁻¹⁰³, resulting in a “vaccinal effect”. In fact, IgE is considered to be an immunostimulant antibody linking the innate response with the adaptive immune response¹⁰⁴ and has been successfully used as adjuvant of cancer vaccines^{101,102,105,106}. This immunoactivation is further increased due to the release of potent immunostimulatory cytokines, such as granulocyte-macrophage colony-stimulating factor (GM-CSF), as well as the release of suppressors of regulatory T-cells (T-regs) by mast cells^{96,107}.

### III. Antibody Production

A. Full-Length Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745, each incorporated herein by reference (see, e.g., Harlow and Lane (Eds.), Antibodies: A Laboratory Manual, Cold Spring Harbor Laboratory, New York, NY, USA (1988); incorporated herein by reference). These antibodies may be polyclonal or monoclonal antibody preparations, monospecific antisera, human antibodies, chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various embodiments can also be synthesized in solution or on a solid support in accordance with conventional techniques.

In an example, a polyclonal antibody is prepared by immunizing an animal with an antigen or a portion thereof and collecting antisera from that immunized animal. The antigen may be altered compared to an antigen sequence found in nature. In some embodiments, a variant or altered antigenic peptide or polypeptide is employed to generate antibodies. Inocula are typically prepared by dispersing the antigenic composition in a physiologically tolerable diluent to form an aqueous composition. Antisera is subsequently collected by methods known in the arts, and the serum may be used as-is for various applications or else the desired antibody fraction may be purified by well-known methods, such as affinity chromatography.

Methods of making monoclonal antibodies are also well known in the art (e.g., U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide, or domain. Resulting antibody-producing B-cells from the immunized animal, or all dissociated splenocytes, are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive. Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after about 2-3 weeks) for the desired reactivity. Fusion procedures for making hybridomas, immunization protocols, and techniques for isolation of immunized splenocytes for fusion are known in the art.

Other techniques for producing monoclonal antibodies include the viral or oncogenic transformation of B-lymphocytes, a molecular cloning approach may be used to generate a nucleic acid or polypeptide, the selected lymphocyte antibody method (SLAM)(see, e.g., Babcook et al., Proc. Natl. Acad. Sci. USA, 93:7843-7848 (1996)), the preparation of combinatorial immunoglobulin phagemid libraries from RNA isolated from the spleen of the immunized animal and selection of phagemids expressing appropriate antibodies, or producing a cell expressing an antibody from a genomic sequence of the cell comprising a modified immunoglobulin locus using Cre-mediated site-specific recombination (see, e.g., U.S. Pat. No. 6,091,001).

Monoclonal antibodies may be further purified using filtration, centrifugation, and various chromatographic methods such as high-performance liquid chromatography (HPLC). Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used in accordance with embodiments include, but are not limited to, interleukin-1 (IL-1), IL-2, IL-4, IL-7, IL-12, interferon-γ (INF-γ), granulocyte-macrophage colony-stimulating factor (GM-CSF), Bacillus Calmette-Guerin (BCG), aluminum hydroxide, muramyl dipeptide (MDP) compounds, muramyl tripeptide phosphatidyl ethanolamine (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, cytokines such as interferon-β (IFN-β), IL-2, or IL-12, or genes encoding proteins involved in immune helper functions, such as B7-1 (CD80) or B7-2 (CD86). A phage-display system can be used to expand antibody molecule populations in vitro.

B. Fully Human Antibody Production

Methods are available for making fully human antibodies. Using fully human antibodies can minimize the immunogenic and allergic responses that may be caused by administering non-human monoclonal antibodies to humans as therapeutic agents. In one embodiment, human antibodies may be produced in a transgenic animal, e.g., a transgenic mouse capable of producing multiple isotypes of human antibodies to protein (e.g., IgG, IgA, and/or IgE) by undergoing V-D-J recombination and isotype switching (Payes et al., Genetic Engineering of Antibody Molecules; in Reviews in Cell Biology and Molecular Medicine, Meyers (Ed.), Wiley-VCH Verlag GmbH & Co., Weinheim, Germany (2015); incorporated herein by reference). Accordingly, this aspect applies to antibodies, antibody fragments, and pharmaceutical compositions thereof, but also non-human transgenic animals, B-cells, host cells, and hybridomas that produce monoclonal antibodies. Applications of human antibodies include, but are not limited to, detect a cell expressing an anticipated protein, either in vivo or in vitro, pharmaceutical preparations containing the antibodies of the present invention, and methods of treating disorders by administering the antibodies.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., Proc. Natl. Acad. Sci. USA 90(6):2551-2555 (1993). In one example, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then crossbred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, International Patent Application Publication Nos. WO 96/33735 and WO 94/02602, which are hereby incorporated by reference in their entirety. Additional methods relating to transgenic mice for making human antibodies are described in U.S. Pat. Nos. 5,545,807; 6,713,610; 6,673,986; 6,162,963; 6,300,129; 6,255,458; 5,877,397; 5,874,299 and 5,545,806; in International Patent Application Publication Nos. WO 91/10741 and WO 90/04036; and in European Patent Nos. EP 546073B1 and EP 546073A1, all of which are hereby incorporated by reference in their entirety for all purposes.

Using hybridoma technology, antigen-specific humanized monoclonal antibodies with the desired specificity can be produced and selected from the transgenic mice such as those described above. Such antibodies may be cloned and expressed using a suitable vector and host cell, or the antibodies can be harvested from cultured hybridoma cells. Fully human antibodies can also be derived from phage-display libraries (as disclosed in, for example, Payes et al., Genetic Engineering of Antibody Molecules; in Reviews in Cell Biology and Molecular Medicine, Meyers (Ed.), Wiley-VCH Verlag GmbH & Co., Weinheim, Germany (2015)). One technique for generating fully human antibodies is described in International Patent Application Publication No. WO1999/010494 (incorporated herein by reference).

C. Antibody Fragments Production

Antibody fragments that retain the ability to recognize the antigen of interest will also find use herein. A number of antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv (see, e.g., Inbar et al., Proc. Nat. Acad. Sci. USA, 69(9):2659-2662 (1972); Hochman et al., Biochem., 15(12):2706-2710 (1976); and Ehrlich et al., Biochem., 19(17):4091-4096 (1980)).

Single-chain variable fragments (scFvs) may be prepared by fusing DNA encoding a peptide linker between DNA molecules encoding the two variable domain polypeptides (V_{L} and V_{H}). scFvs can form antigen-binding monomers, or they can form multimers (e.g., dimers, trimers, or tetramers), depending on the length of a flexible linker between the two variable domains. By combining different V_{L}- and V_{H}-comprising polypeptides, one can form multimeric scFvs that bind to different epitopes. Antigen-binding fragments are typically produced by recombinant DNA methods known to those skilled in the art. Although the two domains of the Fv fragment, V_{L} and V_{H}, are coded for by separate genes, they can be joined using recombinant methods by a synthetic linker that enables them to be made as a single chain polypeptide (known as single chain Fv (sFv or scFv); see e.g., Bird et al., Science, 242(4877):423-426 (1988). Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues, and may include glutamic acid and lysine residues inserted to enhance solubility. Antigen-binding fragments are screened for utility in the same manner as intact antibodies. Such fragments include those obtained by N-terminal and/or C-terminal deletions, where the remaining amino acid sequence is substantially identical to the corresponding positions in the naturally occurring sequence deduced, for example, from a full-length cDNA sequence.

Also contemplated herein are non-peptide compounds having properties analogous to those of a template peptide. These types of non-peptide compounds are termed “peptide mimetics” or “peptidomimetics”.

Also contemplated are “antibody like binding peptidomimetics” (ABiPs), which are peptide-like molecules that act as pared-down antibodies and have certain advantages of longer serum half-life as well as less cumbersome synthesis methods. These analogs can be peptides, non-peptides or combinations of peptide and non-peptide regions. Peptide mimetics that are structurally similar to therapeutically useful peptides may be used to produce a similar therapeutic or prophylactic effect. Such compounds are often developed with the aid of computerized molecular modeling. Generally, peptidomimetics of the disclosure are proteins that are structurally similar to an antibody displaying a desired biological activity, such as the ability to bind a protein, but have one or more peptide linkages optionally replaced by a linkage selected from: —CH2NH—, —CH2S—, —CH2-CH2-, —CH—CH— (cis and trans), —COCH2-, —CH(OH)CH2-, and —CH2SO— by methods well known in the art. Systematic substitution of one or more amino acids of a consensus sequence with a D-amino acid of the same type (e.g., D-lysine in place of L-lysine) may be used in certain embodiments of the disclosure to generate more stable proteins. In addition, constrained peptides comprising a consensus sequence or a substantially identical consensus sequence variation may be generated by methods known in the art (Rizo and Gierasch, Ann. Rev. Biochem., 61:387-418 (1992), incorporated herein by reference), for example, by adding internal cysteine residues capable of forming intramolecular disulfide bridges which cyclize the peptide.

Once generated, a phage display library can be used to improve the immunological binding affinity of Fab molecules using known techniques (see, e.g., Figini et al., J. Mol. Biol., 239(1):68-78 (1994)). The coding sequences for the heavy and light chain portions of the Fab molecules selected from the phage display library can be isolated or synthesized and cloned into any suitable vector or replicon for expression. Any suitable expression system can be used.

### IV. Obtaining Antibodies

In some aspects, there are nucleic acid molecules encoding antibody or antibody-like polypeptides (e.g., heavy or light chain, variable domain only, or full-length). These may be generated by methods known in the art, e.g., isolated from B-cells of mice that have been immunized and isolated, phage display, expressed in any suitable recombinant expression system and allowed to assemble to form antibody molecules.

A. Expression

The nucleic acid molecules may be used to express large quantities of recombinant antibodies or to produce chimeric antibodies, single chain antibodies, antigen-binding fragments, immunoadhesins, diabodies, bispecific antibodies, mutated antibodies, and other antibody derivatives. If the nucleic acid molecules are derived from a non-human, non-transgenic animal, the nucleic acid molecules may be used for antibody humanization.

1. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and/or probes thereof. In addition to control sequences that govern transcription and translation, vectors and expression vectors may contain nucleic acid sequences that serve other functions as well.

To express the antibodies, or antigen-binding fragments thereof, DNA encoding partial or full-length light and heavy chains are inserted into expression vectors such that the gene area is operatively linked to transcriptional and translational control sequences. In some aspects, a vector that encodes a functionally complete human C_{H} or C_{L} immunoglobulin sequence with appropriate restriction sites engineered so that any V_{H} or V_{L} sequence can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

2. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include, but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide using an appropriate expression system.

3. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624; 5,981,274; 5,945,100; 5,780,448; 5,736,524; 5,702,932; 5,656,610; 5,589,466; and 5,580,859, each incorporated herein by reference), including microinjection (U.S. Pat. No. 5,789,215, incorporated herein by reference); by electroporation (U.S. Pat. No. 5,384,253, incorporated herein by reference); by calcium phosphate precipitation; by using DEAE dextran followed by polyethylene glycol; by direct sonic loading; by liposome mediated transfection; by microprojectile bombardment (PCT Publication Nos. WO 94/09699 and 95/06128; U.S. Pat. Nos. 5,610,042; 5,322,783; 5,563,055; 5,550,318; 5,538,877; and 5,538,880, and each incorporated herein by reference); by agitation with silicon carbide fibers U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG mediated transformation of protoplasts (U.S. Pat. Nos. 4,684,611 and 4,952,500, each incorporated herein by reference); by desiccation/inhibition mediated DNA uptake. Other methods include viral transduction, such as gene transfer by lentiviral or retroviral transduction.

4. Host Cells

In another aspect, contemplated are the use of host cells into which a recombinant expression vector has been introduced. Antibodies and antibody-like molecules can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to immune cell (e.g., T-cell) activation. Control of antibody expression allows immune cells, such as tumor-targeting immune cells, to sense their surroundings and perform real-time modulation of cytokine signaling, both in the T-cells themselves and in surrounding endogenous immune cells. One of skill in the art would understand the conditions under which to incubate host cells to maintain them and to permit replication of a vector. Also understood and known are techniques and conditions that would allow large-scale production of vectors and their cognate polypeptides. Host cells which may be used to express antibodies and other antigen-binding proteins of the present disclosure include, for example, murine myeloma cells (e.g., NS0/1 cells, SP2/0-Ag14 cells, and P3X63Ag8.653 cells), Chinese hamster ovary (CHO) cells, baby hamster kidney 21 (BHK21) cells, human embryonic kidney 293 cells (HEK293), fibrosarcoma cells (HT-1080), and the human embryonic retinal cells PER.C6.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

B. Isolation

The nucleic acid molecule encoding either or both of the entire heavy and light chains of an antibody or the variable regions thereof may be obtained from any source that produces antibodies. Methods of isolating mRNA encoding an antibody are well known in the art. The sequences of human heavy and light chain constant region genes are also known in the art. Nucleic acid molecules encoding the full-length heavy and/or light chains may then be expressed in a cell into which they have been introduced and the antibody isolated.

### V. Therapeutic Methods

The compositions of the disclosure may be used for in vivo, in vitro, or ex vivo administration. The route of administration of the composition may be, for example, intracutaneous, subcutaneous, intravenous, local, topical, and intraperitoneal administrations. In some embodiments, the disclosed compositions are used for the treatment, prevention, and/or diagnosis of cancer. The cancer may be a solid tumor, metastatic cancer, non-metastatic cancer, or hematopoietic cancer. In certain embodiments, the cancer may originate in the bone marrow, bone, cartilage, brain, breast, bladder, kidney, ureter, uterus-endometrial, cervix-endocervix, esophagus, stomach, duodenum, small intestine, appendix, cecum, colon, rectum, anal canal, head and neck, salivary glands, thyroid, pancreatobiliary, spleen, liver, lung, oropharynx, larynx, ovary, fallopian tubes, prostate, testis, eye, skin, adipose tissue, synovium, nerve cell/sheath, or thymus.

The cancer may specifically be of one or more of the following tissue origin: glandular epithelium, surface epithelium, fibroblasts, cartilage/bone, striate muscle, smooth muscle, blood vessels, endothelium, fat, neuroectoderm, hepatocytes, and chorionic epithelium. There are different histological types of malignancies (non-epithelial tumors and epithelial tumors). The cancer may specifically be of one or more of the following histological types, though it is not limited to these: liposarcoma, fibrosarcoma, myxosarcoma, chondrosarcoma, osteosarcoma, synovial sarcoma, epithelioid sarcoma, epithelioid angiosarcoma, alveolar soft part sarcoma, malignant fibrous histiocytoma, leiomyosarcoma, rhabdomyosaroma, embryonal rhabdomyosarcoma, alveolar rhabdomyosarcoma, cystosarcoma phyllodes, angiosarcoma, lymphangiosarcoma, invasive meningioma, leukemias, Hodgkin lymphoma (HL) and non-Hodgkin lymphoma (NHL), multiple myeloma (MM) including plasma cell leukemia, mast cell leukemia/sarcoma, erythroleukemia, myeloid leukemia/sarcoma, basophilic leukemia, eosinophilic leukemia, monocytic leukemia, hairy cell leukemia, neurogenic sarcoma, Kaposi's sarcoma, granular cell tumor, gastrointestinal stromal tumor, neuroblastoma, medulloblastoma, retinoblastoma, melanoma including amelanotic, malignant teratomas, primitive neuroectodermal tumor, Ewing's sarcoma, glioblastoma, astrocytoma, neurofibrosarcoma, adamantinoma, chordoma, ependymoma, astrocytoma, oligoendroblastoma, cerebellar sarcoma, germ-cell tumors of ovary and testes (seminoma, dysgerminoma, gynandroblastoma), non-germ cell tumors (embryonic carcinoma, choriocarcinoma, yolk sac tumor, immature teratoma, teratocarcinoma, sex chord-stromal tumors (granulosa cell and Sertoli-Leydig cell tumors). Malignancies also include undifferentiated carcinoma, well-differentiated carcinoma, keratinizing and nonkeratinizing squamous cell carcinoma, basaloid squamous cell carcinoma, NUT midline carcinoma, spindle cell carcinoma, giant cell carcinoma, pleomorphic carcinoma, transitional cell carcinoma, adenocarcinoma, lepidic adenocarcinoma, acinar adenocarcinoma, papillary adenocarcinoma, solid adenocarcinoma, micropapillary adenocarcinoma, mucinous adenocarcinoma, epithelial myoepithelial carcinoma, adenosquamous carcinoma, basal cell carcinoma, large cell carcinoma, large cell neuroendocrine carcinoma, mucoepidermoid carcinoma, adenoid cystic carcinoma, acinic cell carcinoma, hepatocellular carcinoma, cholangiocarcinoma, choriocarcinoma, neuroendocrine carcinoma, lymphoepithelial carcinoma, thymoma, thymic carcinoma, thyroid carcinoma (anaplastic, hurthle cell, papillary, follicular, and medullary thyroid carcinoma), cutaneous squamous cell carcinoma, Paget's disease of the anus, clear cell renal cell carcinoma, cervical carcinoma, urothelial carcinoma, small and non-small cell carcinoma of lung, endometrial adenocarcinoma, adrenocortical carcinoma, chromophobe renal cell carcinoma, granular cell carcinoma, malignant mesothelioma, skin appendages carcinoma (hair, nails, sebaceous glands, sweat glands and mammary glands), Merkel cell carcinoma, pilomatrix carcinoma, apocrine gland carcinoma, papillary eccrine carcinoma, sebaceous adenocarcinoma, mucoepidermoid carcinoma, invasive and noninvasive ductal/lobular carcinoma of breast, inflammatory breast carcinoma, Paget's disease of the breast/nipple and areola, medullary carcinoma, colloid (mucinous) carcinoma including signet ring variant, papillary carcinoma, tubular carcinoma, adenoid cytic carcinoma, secretory carcinoma, carcinoma with metaplasia, ovarian surface epithelium-stroma tumors including serous, mucinous, endometrioid, clear cell, Brunner cells, and transitional cells tumors. In some embodiments, the cancer is breast cancer. In some embodiments, the cancer is triple negative breast cancer (TNBC). In some embodiments, the cancer is HER2/neu positive breast cancer. In some embodiments, the cancer is NHL. In some embodiments, the cancer is MM.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

The selected treatment regimen can be an aggressive one which is expected to result in the best clinical outcome (e.g., complete cure of the disease) or a more moderate one which may relieve symptoms of the disease yet results in incomplete cure of the disease. The type of treatment can include a surgical intervention, administration of a therapeutic drug such as an engineered antibody of the present disclosure, immunotherapy, chemotherapy, an exposure to radiation therapy and/or any combination thereof. The dosage, schedule and duration of treatment can vary, depending on the severity of disease and the selected type of treatment, and those of skill in the art are capable of adjusting the type of treatment with the dosage, schedule and duration of treatment.

Biomarkers like CD128, CD38, and TfR1 that can predict the efficacy of certain therapeutic regimen and can be used to identify patients who will receive benefit of a conventional single or combined modality therapy before treatment begins or to modify or design a future treatment plan after treatment. In the same way, those patients who do not receive much benefit from such conventional single or combined modality therapy and can offer them alternative treatment(s) may be identified.

### VI. Immunotherapy

In some embodiments, the methods of the disclosure comprise administration of a cancer immunotherapy. Cancer immunotherapies can be categorized as active, passive, or hybrid (active and passive). These approaches exploit the fact that cancer cells often have molecules on their surface that can be detected by the immune system; they are often proteins or other macromolecules (e.g. carbohydrates). Active immunotherapy directs the immune system to attack tumor cells by targeting tumor antigens. Passive immunotherapies enhance existing anti-tumor responses and include the use of monoclonal antibodies, lymphocytes and cytokines. Various immunotherapies are known in the art, and some are described below.

A. Checkpoint Inhibitors

Embodiments of the disclosure may include administration of immune checkpoint inhibitors, which are further described below.

1. PD-1, PDL1, and PDL2 Inhibitors

PD-1 can act in the tumor microenvironment where T-cells encounter an infection or tumor. Activated T-cells upregulate PD-1 and continue to express it in the peripheral tissues. Cytokines such as IFNγ induce the expression of PDL1 (also “PD-L1”) on epithelial cells and tumor cells. PDL2 (also “PD-L2”) is expressed on macrophages and dendritic cells. The main role of PD-1 is to limit the activity of effector T-cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab (Opdivo®), pembrolizumab (Keytruda®), and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab (Opdivo®), also known as MDX-1106-04, MDX-1106, ONO-4538, and BMS-936558, is an anti-PD-1 antibody described in WO2006/121168. Pembrolizumab (Keytruda®), also known as MK-3475, Merck 3475, lambrolizumab, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab (Opdivo®), pembrolizumab (Keytruda®), or pidilizumab. Accordingly, in one embodiment, the inhibitor comprises the CDR1, CDR2, and CDR3 domains of the V_{H} region of nivolumab (Opdivo®), pembrolizumab (Keytruda®), or pidilizumab, and the CDR1, CDR2 and CDR3 domains of the V_{L} region of nivolumab (Opdivo®), pembrolizumab (Keytruda®), or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

2. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T-cells and acts as an “off” switch when bound to B7-1 (CD80) or B7-2 (CD86) on the surface of antigen-presenting cells. CTLA-4 is a member of the immunoglobulin superfamily that is expressed on the surface of helper T-cells and transmits an inhibitory signal to T-cells. CTLA-4 is similar to the T-cell co-stimulatory protein, CD28, and both molecules bind to B7-1 and B7-2 on antigen-presenting cells. CTLA-4 transmits an inhibitory signal to T-cells, whereas CD28 transmits a stimulatory signal. Intracellular CTLA-4 is also found in regulatory T-cells and may be important to their function. T-cell activation through the T-cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or V_{H} and/or V_{L} domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129; International Patent Application Nos. WO 01/14424, WO 98/42752, and WO 00/37504 (CP675,206, also known as tremelimumab; formerly ticilimumab), U.S. Pat. No. 6,207,156: Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application Nos. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (Yervoy®), also known as 10D1, MDX-010, and MDX-101 or antigen binding fragments and variants thereof (see, e.g., International Patent Application No. WO 01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the V_{H} region of tremelimumab or ipilimumab, and the CDR1, CDR2 and CDR3 domains of the V_{L} region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

B. Activation of Co-stimulatory Molecules

In some embodiments, the immunotherapy comprises an agonist of a co-stimulatory molecule. In some embodiments, the agonist comprises an activator of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Agonists include agonistic antibodies, polypeptides, compounds, and nucleic acids. Various agonists of co-stimulatory molecules are recognized in the art, certain examples of which are described in Mayes et al., Nat. Rev. Drug. Discov., 17(7):509-527 (2018), incorporated herein by reference in its entirety.

C. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen-presenting cells in the mammalian immune system. In cancer treatment they aid cancer antigen targeting. One example of cellular cancer therapy based on dendritic cells is sipuleucel-T (Provenge®).

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as toll-like receptor 3 (TLR3), TLR7, TLR8 or CD40 have been used as antibody targets.

Various dendritic cell therapies are recognized in the art, certain examples of which are described in Sadeghzadeh et al., Life Sci., 254: Article117580 (2020), incorporated herein by reference in its entirety.

D. CAR Therapy

Chimeric antigen receptors (CARs, also known as chimeric immunoreceptors, chimeric T-cell receptors or artificial T-cell receptors) are engineered receptors that combine a new specificity with an immune cell to target cancer cells. Typically, these receptors graft the specificity of a monoclonal antibody onto a T-cell or other immune cell (e.g., NK cell, NKT cell, macrophage, etc.). The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T-cell has been engineered to become a CAR-T cell, it acts as a “living drug”. CAR-T cells create a link between an extracellular ligand recognition domain to an intracellular signaling molecule which in turn activates T-cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include tisagenlecleucel (Kymriah®) and axicabtagene ciloleucel (Yescarta®).

Various CAR cell therapies are recognized in the art, certain examples of which are described in Britten et al., Nat. Rev. Drug Discov., 20(6):476-488 (2021); Hong et al., Cancer Cell 38(4):473-488 (2020); and Xie et al., EBioMedicine 59: Article 102975 (2020), each incorporated herein by reference in its entirety.

E. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

Various cytokine therapies are recognized in the art, certain examples of which are described in Qiu et al., Drug Des. Devel. Ther., 15:2269-2287 (2021), incorporated herein by reference in its entirety.

F. Adoptive T-Cell Therapy

Adoptive T-cell therapy is a form of passive immunization by the transfusion of T-cells (adoptive cell transfer). They are found in blood and tissue and usually activate when they find foreign pathogens. Specifically, they activate when the T-cell's surface receptors encounter cells that display parts of foreign proteins on their surface antigens. These can be either infected cells, or antigen-presenting cells. They are found in normal tissue and in tumor tissue, where they are known as tumor infiltrating lymphocytes (TILs). They are activated by the presence of antigen-presenting cells such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T-cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

Various adoptive T-cell therapies are recognized in the art, certain examples of which are described in Yang et al., Adv. Immunol. 130:279-297 (2016), incorporated herein by reference in its entirety.

### VII. Administration of Therapeutic Compositions

The therapy provided herein may comprise administration of a combination of therapeutic agents, such as a first cancer therapy (e.g., an engineered antibody disclosed herein) and a second cancer therapy (e.g., radiotherapy, chemotherapy, immunotherapy, etc.). The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3,4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/mi or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VII. General Pharmaceutical Compositions

In some embodiments, pharmaceutical compositions are administered to a subject. Different aspects may involve administering an effective amount of a composition to a subject. In some embodiments, an antibody or antigen-binding fragment capable of binding to CD138, CD38, or TfR1 (e.g., an IgE antibody disclosed herein) is administered to the subject to protect against or treat a condition (e.g., cancer). Alternatively, an expression vector encoding one or more such antibodies or polypeptides or peptides may be given to a subject as a preventative treatment. Additionally, such compositions can be administered in combination with an additional therapeutic agent (e.g., a chemotherapeutic, immunotherapeutic). Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

### IX. Kits

Certain aspects of the present invention also concern kits containing compositions of the invention or compositions to implement methods of the invention. In some embodiments, kits can be used to evaluate one or more biomarkers. In some embodiments, kits can be used to detect one or more proteins. In some embodiments, the disclosed kits are used to detect one or more of CD38, CD138, and TfR1 in a sample. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, a kit contains at least or contains at most, 1, 2, 3, 4, 5, 6, 7, 8, 9, or 10 engineered antibodies. In some embodiments, a kit contains an anti-CD138 engineered antibody. In some embodiments, a kit contains an anti-CD38 engineered antibody. In some embodiments, a kit contains an anti-TfR1 engineered antibody.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, 20×, or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

Any embodiment of the disclosure involving a specific biomarker by name is contemplated also to cover embodiments involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

Embodiments of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide, poly(A) polymerase, and poly(A) polymerase buffer. Labeling reagents can include an amine-reactive dye.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—Construction and Expression of IgE Antibodies

### Methods

Three novel antibodies of the IgE class were developed using the human a heavy chain and targeting the human cancer antigens CD138, CD38, and TfR1. To develop the anti-CD38 IgE, the amino acid sequences of the V_{H} and V_{L} of the fully human antibody daratumumab were used (SEQ ID NO:17 and SEQ ID NO:18, respectively)¹⁰⁸. To develop the anti-CD138 IgE, the amino acid sequences of the heavy (H) and light (L) chain variable (V) regions (V_{H} and V_{L}) of the murine anti-CD138 IgG1 monoclonal antibody B-B4¹⁰⁹ were used (SEQ ID NO:7 and SEQ ID NO:8, respectively)¹⁰⁹. To develop the anti-TfR1 IgE antibody, the amino acid sequences of the anti-TfR1 IgG1 murine monoclonal antibody 128.1 were used (SEQ ID NO:25 and SEQ ID NO:26, respectively)¹¹⁰⁻¹¹². Vectors comprising human light chain κ (SEQ ID NO:10) and heavy chain ε (SEQ ID NO:9) were used to clone and express the anti-CD138 IgE (mouse/human chimeric antibody), anti-CD38 IgE (fully human antibody), and anti-TfR1 IgE (mouse/human chimeric antibody). These IgE class antibodies and their respective IgG1 (human γ1 heavy chain) counterparts were expressed in mammalian cells (CHO cells or murine myeloma cells such as Sp2/0-Ag14, P3X63Ag8.653, or NS0/1 cells), which were grown in roller bottles and the antibodies purified from cell culture supernatant by affinity chromatography and molecular weight (m.w.) and assembly assessed by sodium dodecyl sulfate-polyacrylamide gel electrophoresis (SDS-PAGE)^{102,103}.

### Results

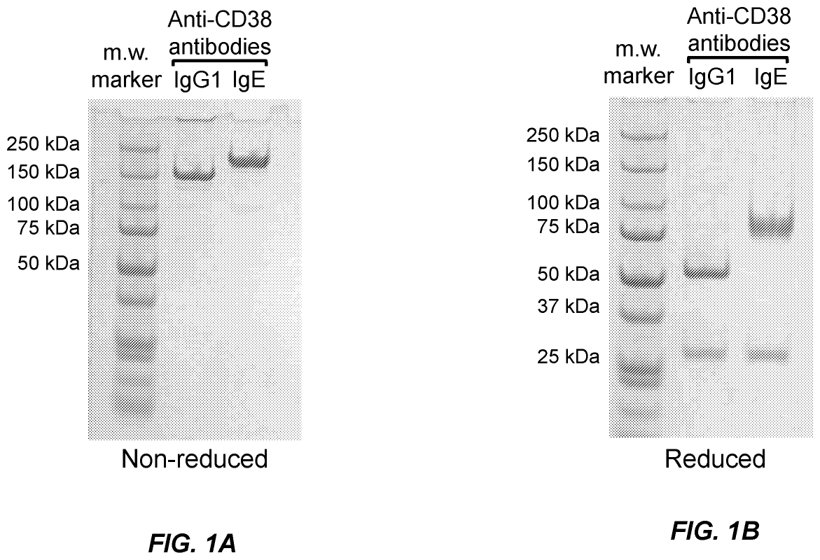

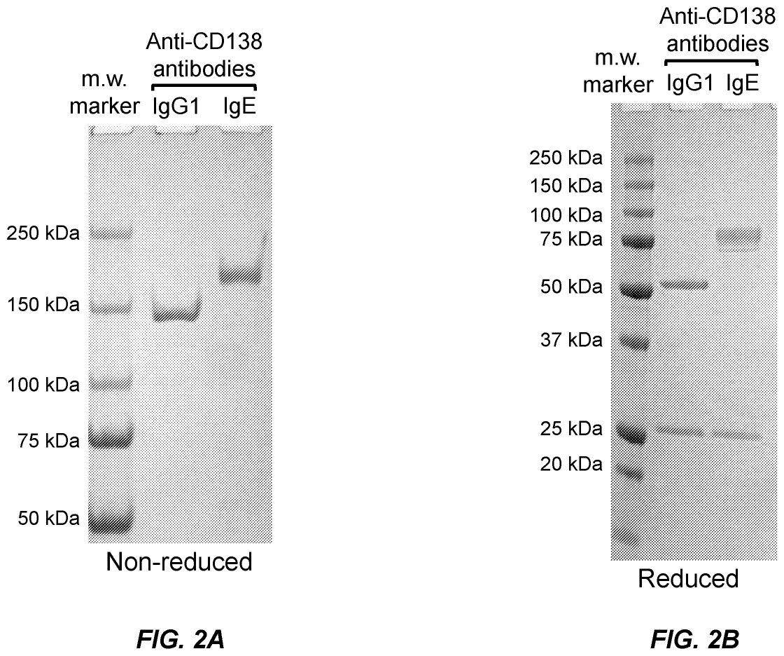

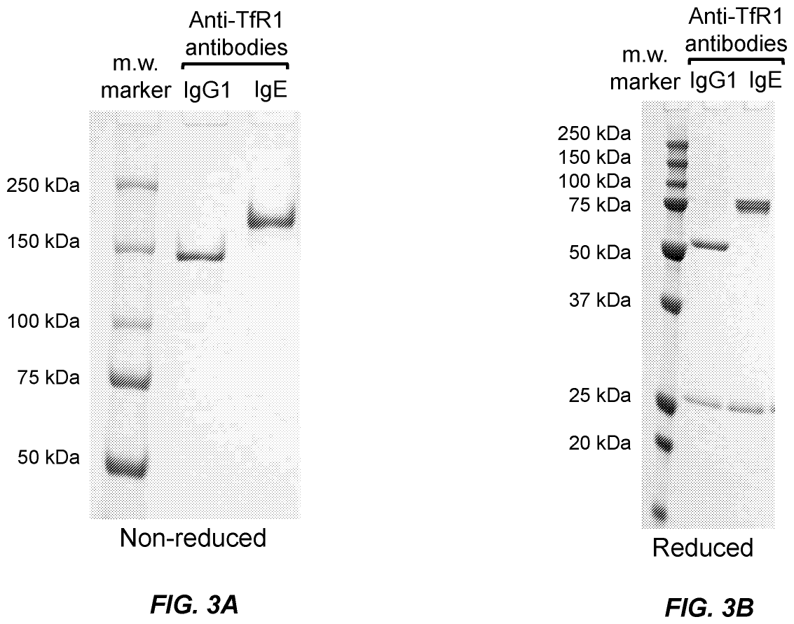

The three IgE antibodies and their respective IgG1 counterparts exhibited the correct m.w. and were properly assembled. FIGS. 1A-1B, 2A-2B, and 3A-3B show SDS-PAGE analysis of the anti-CD38 IgE, anti-CD138 IgE, and anti-TfR1 IgE antibodies, respectively. This analysis demonstrates the expected m.w. of approximately 150 kDa of human IgG1 and the higher m.w. of human IgE (approximately 190 kDa) due to the increased mass of its heavy chain as shown in FIGS. 1B, 2B, and 3B. This increase in mass is explained by the presence of an additional domain of the IgE (Cε4) that increases its m.w.^{3,7,12}

### Example 2—Binding to Antigen and FcεRI

### Methods

Antigen binding of the anti-CD38 IgE, anti-CD138 IgE, and anti-TfR1 IgE antibodies was studied by flow cytometry using the human multiple myeloma (MM) cell line MM.1S purchased from the American Type Culture Collection (ATCC, Manassas, VA, USA; ATCC® CRL-2974™). In addition, antigen binding of anti-CD138 IgE was also studied by flow cytometry using the human triple negative breast cancer (TNBC) cell line MDA-MB-468 (ATCC® HTB-132™) and the human HER2/neu positive breast cancer cell line SK-BR-3 (ATCC® HTB-30™). Binding to FcεRI was also assessed by flow cytometry using the rat basophilic leukemia cell line RBL SX-38 expressing human FcεRI¹¹³ kindly provided by Dr. Jean-Pierre Kinet (Beth Israel Deaconess Medical Center, Boston, MA, USA). Cells (5×10⁵) were incubated with either 2 μg of anti-CD38 IgE, anti-CD138 IgE, or anti-TfR1 IgE; anti-CD38 IgG1 or anti-CD138 IgG1; or IgE isotype control. The latter is a mouse/human chimeric IgE specific for the hapten DNS (5-dimethylamino naphthalene-1-sulfonyl chloride), also named dansyl¹¹⁴ Antibody binding was detected using a phycoerythrin (PE)-conjugated goat F(ab′)₂ anti-human κ antibody (SouthernBiotech, Birmingham, AL, USA) for the IgG1 and IgE antibodies targeting CD38 and CD138 or using a PE-conjugated mouse IgG1 anti-human IgE antibody (ThermoFisher Scientific, Waltham, MA, USA) for IgE antibody targeting TfR1. Samples were analyzed on a BD LSRII analytical flow cytometer (BD Biosciences, San Jose, CA, USA) and 10,000 events were collected. Histograms were created using the FSC Software Version 3 (De Novo Software, Glendale, CA, USA).

### Results

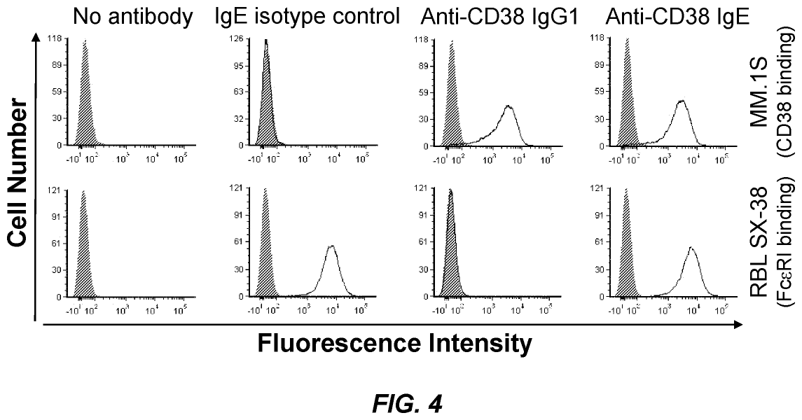

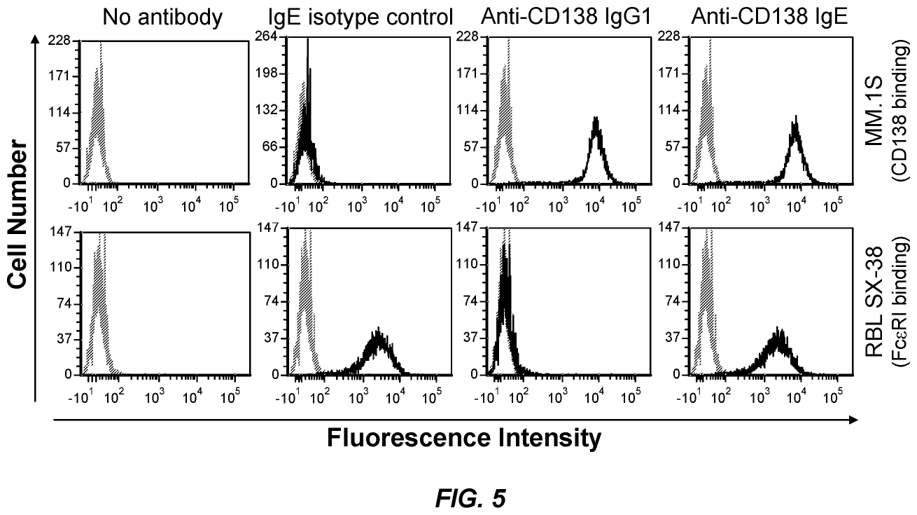

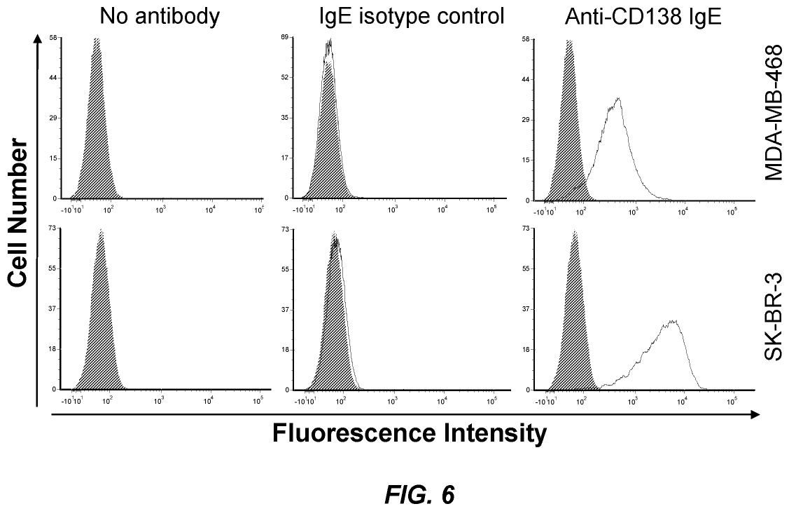

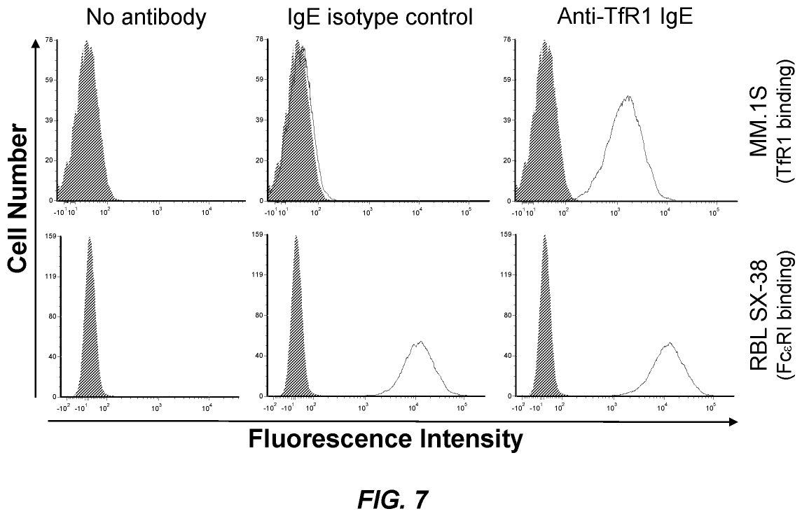

FIGS. 4-7 show that the anti-CD38 IgE, anti-CD138 IgE, and anti-TfR1 IgE antibodies bind their respective human antigens expressed on malignant cells and also bind human FcεRI consistent with their Fc IgE regions.

### Example 3—In Vitro Degranulation Assay

### Methods

The rat basophilic leukemia cells RBL SX-38 expressing human FcεRI¹¹³ (2.5×10⁵ in 0.5 ml per well in a 24 well tissue culture plate seeded the day before) were incubated with 1 μg of either IgE isotype control; anti-CD38 IgE, anti-CD138 IgE, or anti-TfR1 IgE; or anti-CD38 IgG1, anti-CD138 IgG1, or anti-TfR1 IgG1; with or without malignant cells expressing the antigen. The human MM cell line MM.1S was used for all antibodies and the human breast cancer cell lines MDA-MB-468 and SK-BR-3 were used for the anti-CD138 IgE. Degranulation releases β-hexosaminidase into the cell culture media, which is measured via an enzymatic colorimetric assay. Percentage (%) of degranulation was determined by comparison to total β-hexosaminidase released after membrane solubilization by 1% Triton X-100 (Sigma-Aldrich, St. Louis, MO, USA) in PBS.

### Results

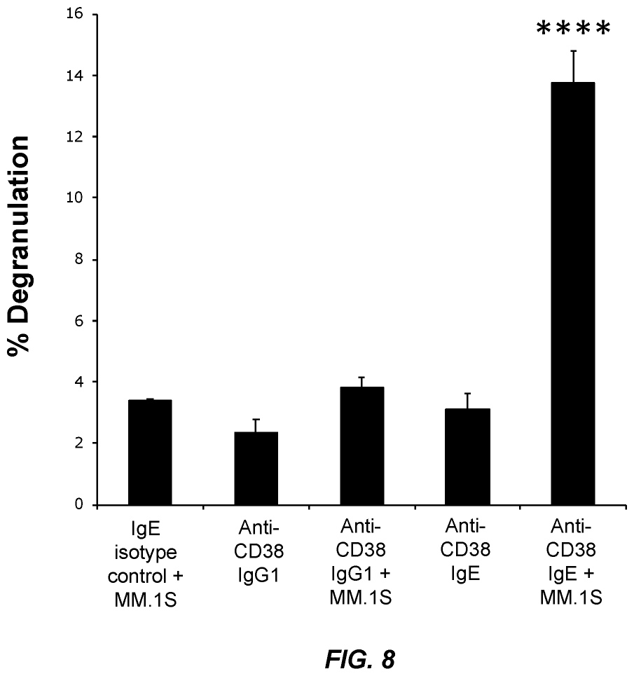

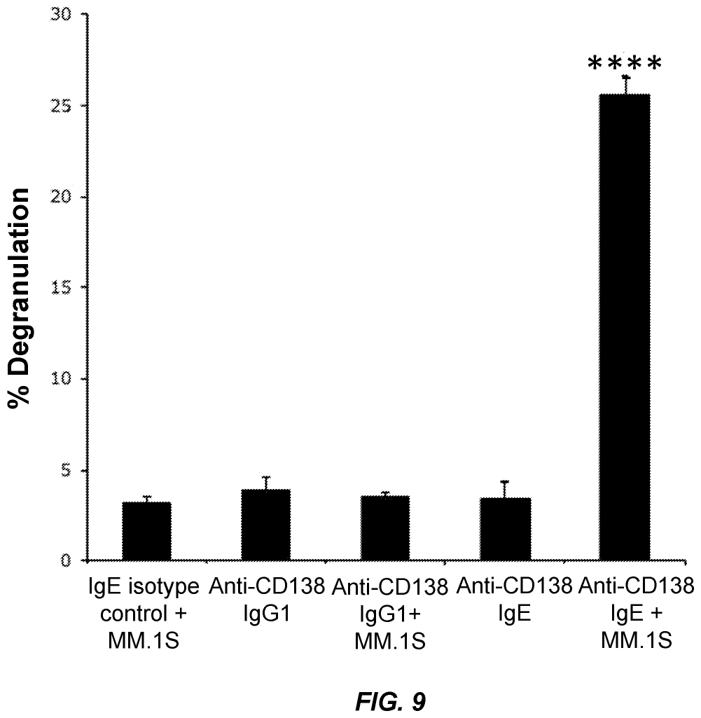

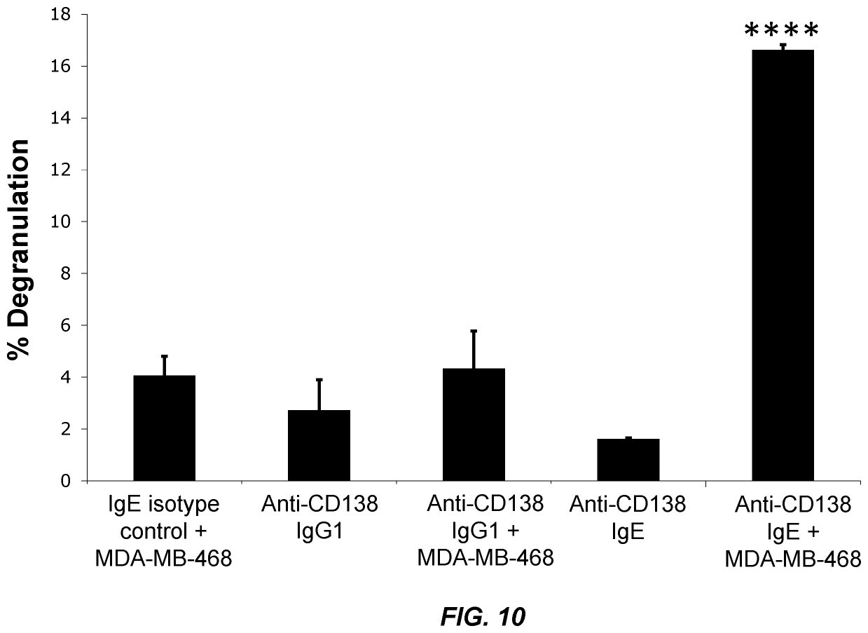

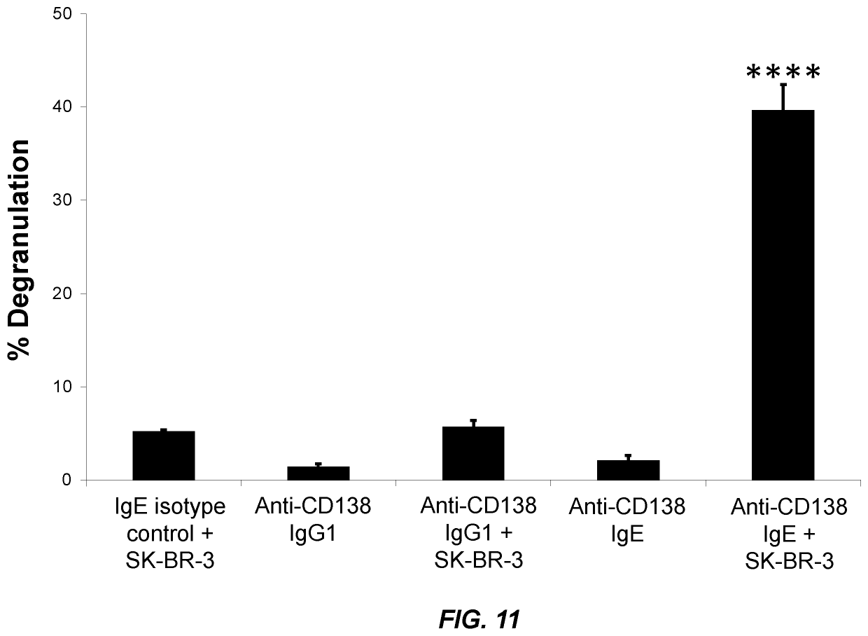

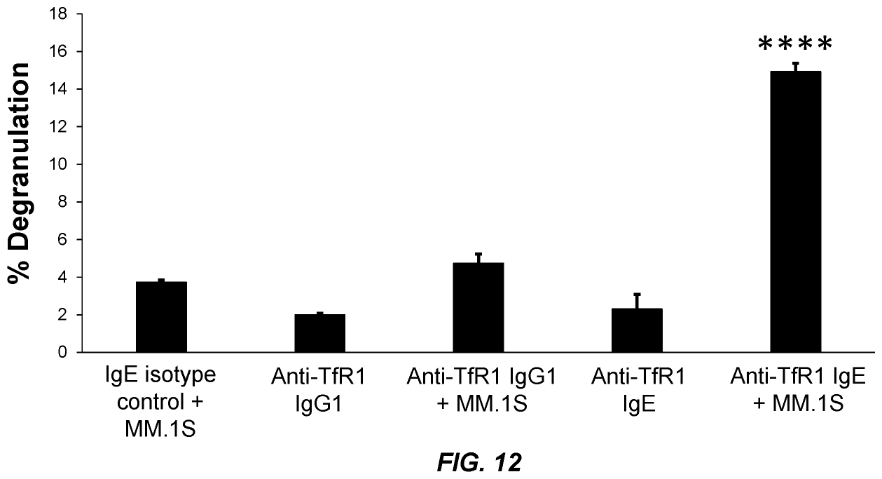

FIGS. 8-12 show that the anti-CD38 IgE, anti-CD138 IgE, and anti-TfR1 IgE antibodies trigger in vitro degranulation of basophilic cells expressing FcεRI in the presence of cancer cells expressing the antigen. FIGS. 8-12 also show that the anti-CD38 IgG1, anti-CD138 IgG1, and anti-TfR1 IgG1 did not trigger degranulation, showing only a basal level release since this reaction is IgE specific. These results demonstrate that the IgE antibodies are functional and further suggest that an acute IgE inflammatory response would occur in the tumor microenvironment, in which the tumor antigen is expressed at a density on the surface of cancer cells sufficient to trigger FcεRI cross-linking and degranulation of tumor infiltrating cells, such as mast cells, capable of eliciting anti-tumor activity.

### Example 4—Passive Cutaneous Anaphylaxis (PCA) Assay

### Methods

The ability of anti-CD38 IgE, anti-CD138 IgE, and anti-TfR1 IgE antibodies to induce passive cutaneous anaphylaxis, a Type I hypersensitivity reaction, was assessed in transgenic mice expressing a humanized FcεRI mice (huFcεRI mice), which is required since human IgE is not recognized by murine FcεRI¹¹⁵. Mice were injected intradermically (i.d.) with buffer (PBS) or 1-5 μg/ml of either anti-CD38 IgE, anti-CD138 IgE, or anti-TfR1 IgE; or the same amount of anti-CD38 IgG1, anti-CD138 IgG1, or anti-TfR1 IgG1 in a volume of 50 μL. After at least 1 hour, 25 μg of a goat anti-human κ antibody (Sigma-Aldrich) was injected intravenously (i.v.) in 1% Evans blue (Bio-Techne, Minneapolis, MN, USA) in PBS. Mice were then euthanized in approximately 30 minutes. Cutaneous anaphylaxis was assessed visually by the blue dye leakage from blood vessels into the skin due to vasodilation.

### Results

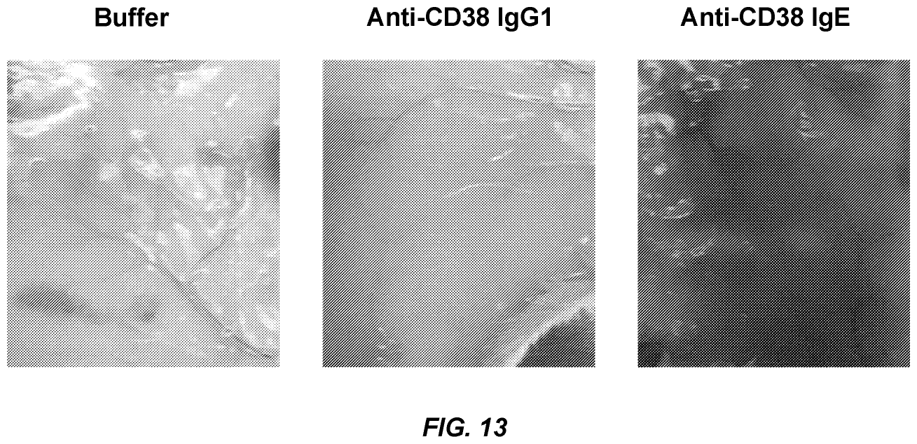

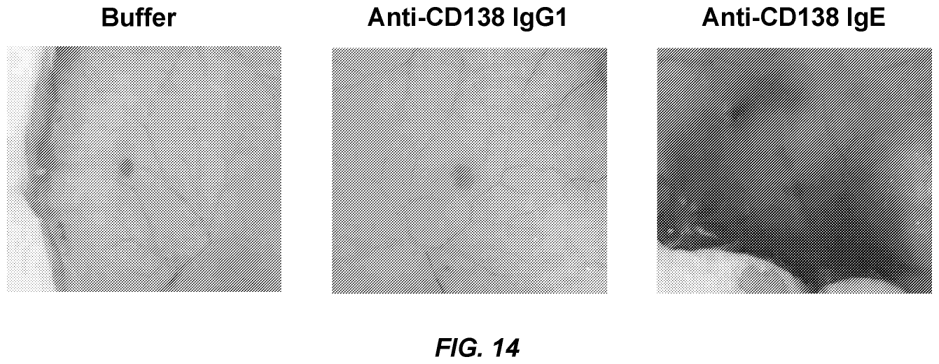

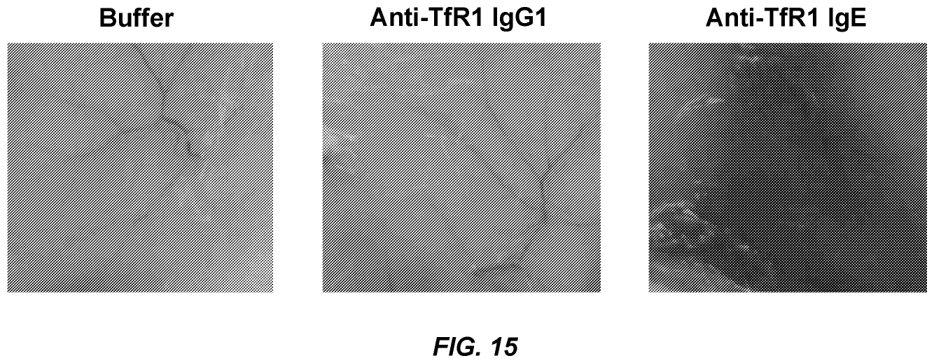

FIGS. 13-15 show that leakage (extravasation) of the blue dye into the skin of huFcεRI mice in the PCA assay was observed only when the anti-CD38 IgE, anti-CD138 IgE, or anti-TfR1 IgE, but not their IgG1 counterparts, were artificially cross-linked with a secondary antibody (anti-human κ) on the surface of mast cells demonstrating that three IgE antibodies are fully functional in vivo and suggesting that these antibodies are capable of eliciting anti-tumor activity in vivo. The ability of IgE to increase local vascular permeability, a “gatekeeper” IgE effect, facilitates tumor penetration of diagnostic or therapeutic agents, including soluble molecules and cells, that would better target the tumor 3. This is per se, another application of IgE in combination therapy.

### Example 5—Induction of Antibody-Dependent Cell-Mediated Cytotoxicity (ADCC) and Antibody-Dependent Cell-Mediated Phagocytosis (ADCP)

### Methods

Monocytes isolated from peripheral blood mononuclear cells (PBMC) using EasySep™ Human Monocyte Isolation Kit (STEMCELL Technologies, Inc., Vancouver, BC, Canada) were incubated with AIM V (Gibco Life Technologies, Grand Island, NY, USA)+5% of fetal bovine serum (FBS) (R&D Systems, Flowery Branch, GA, USA) and 10 ng/ml of human interleukin-4 (IL-4)(Peprotech, Rocky Hill, NJ, USA) for 20 hours to induce expression of FcεRII and then used to assess ADCC/ADCP by three-color flow cytometry as reported for IgE and IgG antibodies^{98,116,117}. Monocytes were also differentiated towards M1 activated macrophages using ImmunoCult™-SF macrophage medium (STEMCELL Technologies, Inc.) following manufacturer instructions. Human MM cells MM.1S (0.1 million) labeled with carboxyfluorescein succinimidyl ester (CFSE) (ThermoFisher Scientific, Waltham, MA, USA) were incubated with 0.5 million monocytes or M1 activated macrophages (5:1 effector-to-target ratio) in the presence of buffer or 5 μg/ml of IgE isotype control, anti-CD38 IgE, anti-CD138 IgE, or anti-TfR1 IgE at 37° C. for 2.5 hours. Cells were washed and incubated with PE-conjugated anti-human CD89 (BD Biosciences Franklin Lakes, NJ, USA) antibody to label monocytes or macrophages and with 4′,6-diamidino-2-phenylindole (DAPI) to identify dead cells. CFSE⁺/PE⁺ cells designate the occurrence of ADCP and CFSE⁺/DAPI⁺ cells designate the occurrence of ADCC. Cells treated with 0.3% saponin (MilliporeSigma, St. Louis, MO, USA) in PBS were used as positive control for dead cells (100% killing).

### Results

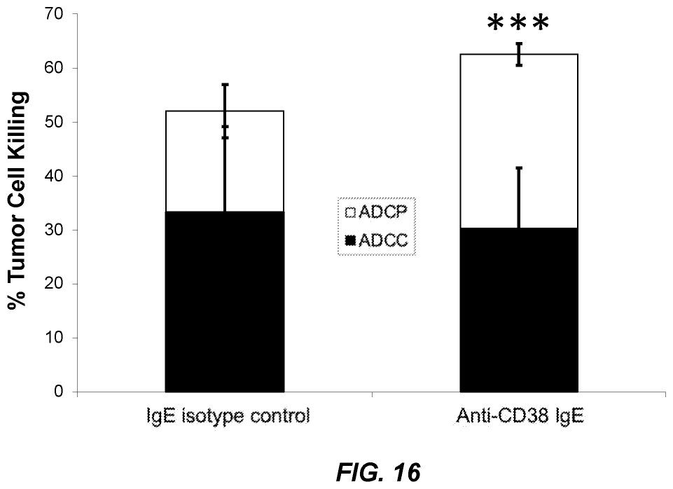

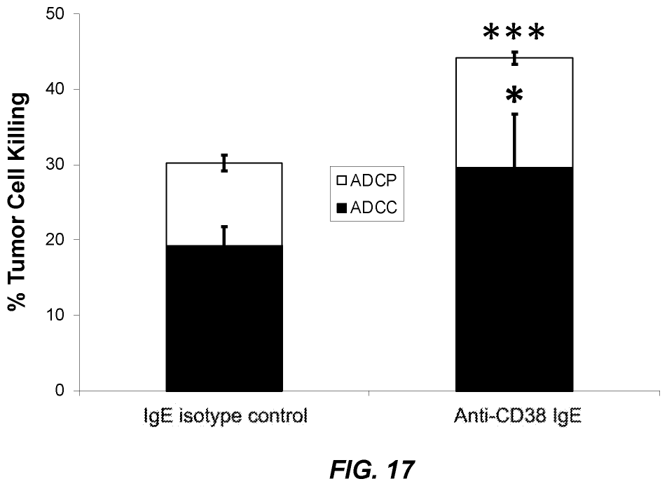

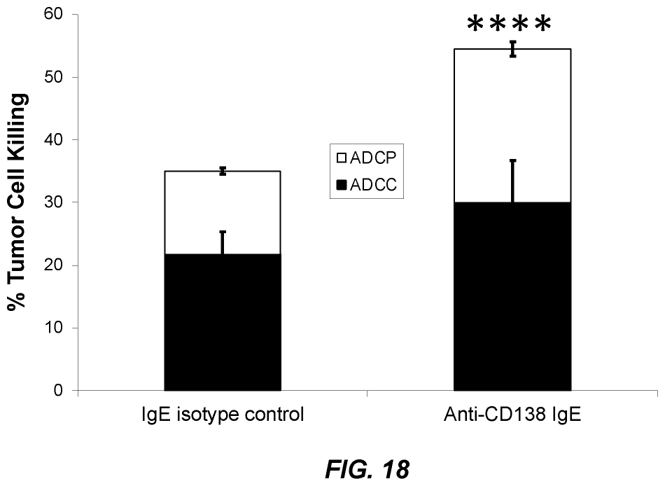

FIGS. 16 and 18 show that the anti-CD38 IgE and anti-CD138 IgE antibodies in the presence of monocytes treated with IL-4 as effector cells showed significant enhancement of ADCP (p<0.001, Student's 1-test) against MM.1S target cells compared to the IgE isotype control. The results of incubation with buffer control and those of IgE isotype control were similar (not shown). In addition, FIG. 17 shows that the anti-CD38 IgE antibody in the presence of M1 activated macrophages as effector cells showed significant enhancement of ADCP (p<0.001, Student's t-test) and ADCC (p<0.05, Student's t-test) against MM.1S target cells compared to the IgE isotype control. The results of incubation with buffer control and those of IgE isotype control were similar (not shown). Taken together, these results suggest that monocytes and macrophages armed with these tumor-targeting IgE antibodies would trigger anti-tumor activity in vivo.

### Example 6—In Vivo Efficacy in a Disseminated Xenograft Mouse Model of Human MM

### Methods

Female C.B-17 severe combined immune deficiency (SCID)-Beige mice 8 to 12 weeks old were exposed to 3 gray (Gy) total-body sublethal irradiation (GammaCell40 irradiator ¹³⁷Cs, Best Theratronics, Ltd., Ottawa, ON, Canada) on the day before tumor challenge (Day −1). MM.1S human MM cells (5×10⁶) in Hank's balanced salt solution (HBSS) were injected i.v. via the tail vein as reported^{111,117}. Mice were randomized into treatment groups and treatments were given via tail vein injection on Day 1 and Day 7 after tumor challenge. Mice were treated with buffer (PBS) control, 100 μg of anti-CD38 IgE, 5×10⁶ PBMC, or 5×10⁶ PBMC combined with 100 μg of anti-CD38 IgE. PBMC provides monocytes as IgE effector cells, which are necessary since human IgE is not recognized by murine FcεRI¹¹⁵. Survival was based on the time from tumor challenge to the development of hind-limb paralysis, when mice were euthanized. Survival plots were generated using GraphPad Prism Version 4. Median survival and differences in survival (log-rank test) were determined using the same software.

### Results

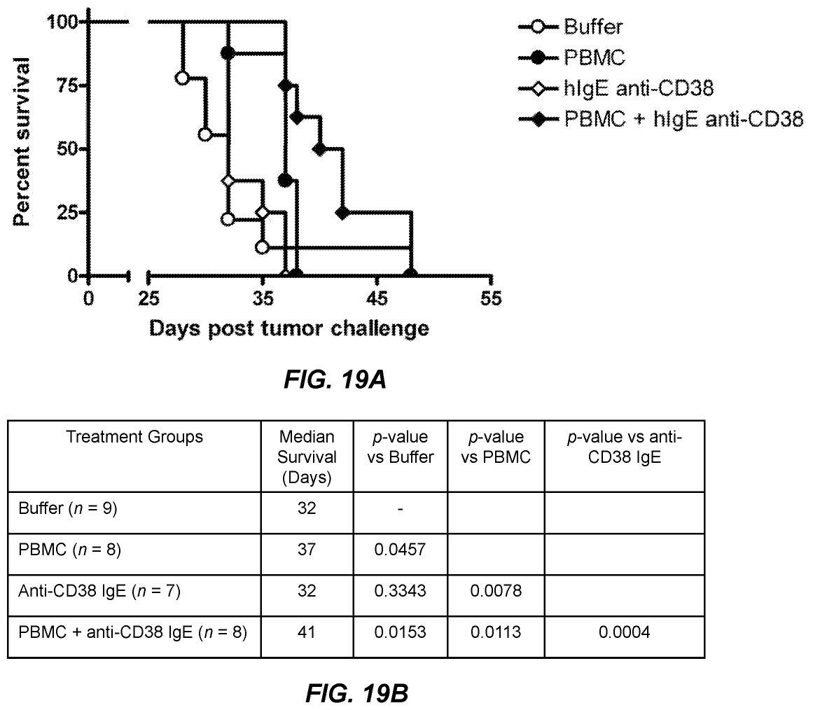

FIGS. 19A-1B show that treatment of mice with anti-CD38 IgE+PBMC significantly (p<0.05, log-rank test) prolonged the survival compared to all other treatments. This result is particularly relevant given the limitation in type and number of effector cells administered into the SCID-Beige mice and suggests that the new IgE antibodies such as anti-CD38 IgE would be effective as cancer therapeutics.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, and those cited herein, to the extent that they provide exemplary procedural or other details supplementary to those set forth herein, are specifically incorporated herein by reference.
1 Vernersson, M., Aveskogh, M. & Hellman, L. Cloning of IgE from the echidna (Tachyglossus aculeatus) and a comparative analysis of epsilon chains from all three extant mammalian lineages. Dev Comp Immunol 28, 61-75, (2004).
2 Erazo, A., Kutchukhidze, N., Leung, M., Christ, A. P., Urban, J. F., Jr., Curotto de Lafaille, M. A. & Lafaille, J. J. Unique maturation program of the IgE response in vivo. Immunity 26, 191-203, (2007).
3 Daniels, T. R., Rodriguez, J. A., Ortiz-Sanchez, E., Helguera, G. & Penichet, M. L. The IgE antibody and its use in cancer immunotherapy. In “Cancer and IgE: Introducing the Concept of AllergoOncology”. Penichet M. L. and Jensen-Jarolim E., eds. Springer, New York, USA. 2010, pp. 159-83.
4 Kinet, J. P. The high-affinity IgE receptor (Fc epsilon RI): from physiology to pathology. Annu Rev Immunol 17, 931-972, (1999).
5 Adamczewski, M. & Kinet, J. P. The high-affinity receptor for immunoglobulin E. Chem. Immunol. 59, 173-190, (1994).
6 Achatz, G., Achatz-Straussberger, G., Feichtner, S., Konigsberger, S., Lenz, S., Peckl-Schmid, D., Zaborsky, N. & Lamers, M. The Biology of IgE: Molecular mechanism restraining potentially dangerous high serum IgE titres in vivo. In “Cancer and IgE: Introducing the Concept of AllergoOncology”. Penichet M. L. and Jensen-Jarolim E., eds. Springer, New York, USA. 2010, pp. 13-36.
7 Payes, C. J., Daniels-Wells, T. R., Maffia, P., Penichet, M. L., Morrison, S. L. & Helguera, G. Genetic Engineering of Antibody Molecules. In Reviews in Cell Biology and Molecular Medicine. Robert A. Meyers, ed. Wiley-VCH Verlag GmbH & Co., Weinheim, Germany. New edition. 2015, Vol. 1, No. 3, pp. 1-52.
8 Mukai, K., Tsai, M., Saito, H. & Galli, S. J. Mast cells as sources of cytokines, chemokines, and growth factors. ImmunolRev 282, 121-150, (2018).
9 Jensen-Jarolim, E., Bax, H. J., Bianchini, R., Capron, M., Corrigan, C., Castells, M., Dombrowicz, D., Daniels-Wells, T. R., Fazekas, J., Fiebiger, E., Gatault, S., Gould, H. J., Janda, J., Josephs, D. H., Karagiannis, P., Levi-Schaffer, F., Meshcheryakova, A., Mechtcheriakova, D., Mekori, Y., Mungenast, F., Nigro, E. A., Penichet, M. L., Redegeld, F., Saul, L., Singer, J., Spicer, J. F., Siccardi, A. G., Spillner, E., Turner, M. C., Untersmayr, E., Vangelista, L. & Karagiannis, S. N. AllergoOncology—the impact of allergy in oncology: EAACI position paper. Allergy 72, 866-887, (2017).
10 Martinez-Maza, O., Moreno, A. D. & Cozen, W. Epidemiological Evidence: IgE, Allergies, and Hematopoietic Malignancies. In “Cancer and IgE: Introducing the Concept of AllergoOncology”. Penichet M. L. and Jensen-Jarolim E., eds. Springer, New York, USA. 2010, pp. 79-136.
11 Lowcock, E. C., Cotterchio, M. & Ahmad, N. Association between allergies, asthma, and breast cancer risk among women in Ontario, Canada. Cancer Causes Control 24, 1053-1056, (2013).
12 Leoh, L. S., Daniels-Wells, T. R. & Penichet, M. L. IgE immunotherapy against cancer. Curr Top Microbiol Immunol 388, 109-149, (2015).
13 Fu, S. L., Pierre, J., Smith-Norowitz, T. A., Hagler, M., Bowne, W., Pincus, M. R., Mueller, C. M., Zenilman, M. E. & Bluth, M. H. Immunoglobulin E antibodies from pancreatic cancer patients mediate antibody-dependent cell-mediated cytotoxicity against pancreatic cancer cells. Clin Exp Immunol 153, 401-409, (2008).
14 Matta, G. M., Battaglio, S., Dibello, C., Napoli, P., Baldi, C., Ciccone, G., Coscia, M., Boccadoro, M. & Massaia, M. Polyclonal immunoglobulin E levels are correlated with hemoglobin values and overall survival in patients with multiple myeloma. Clin Cancer Res 13, 5348-5354, (2007).
15 Ferastraoaru, D., Gross, R. & Rosenstreich, D. Increased malignancy incidence in IgE deficient patients not due to concomitant Common Variable Immunodeficiency. Ann Allergy Asthma Immunol 119, 267-273, (2017).
16 Ferastraoaru, D. & Rosenstreich, D. IgE deficiency and prior diagnosis of malignancy: Results of the 2005-2006 National Health and Nutrition Examination Survey. Ann Allergy Asthma Immunol 121, 613-618, (2018).
17 Adachi, M., Kai, K., Yamaji, K., Ide, T., Noshiro, H., Kawaguchi, A. & Aishima, S. Transferrin receptor 1 overexpression is associated with tumour de-differentiation and acts as a potential prognostic indicator of hepatocellular carcinoma. Histopathology 75, 63-73, (2019).
18 Shen, Y., Li, X., Zhao, B., Xue, Y., Wang, S., Chen, X., Yang, J., Lv, H. & Shang, P. Iron metabolism gene expression and prognostic features of hepatocellular carcinoma. J Cell Biochem 119, 9178-9204, (2018).
19 Palaiologou, M., Delladetsima, I. & Tiniakos, D. CD138 (syndecan-1) expression in health and disease. Histol Histopathol 29, 177-189, (2014).
20 Moreaux, J., Sprynski, A. C., Dillon, S. R., Mahtouk, K., Jourdan, M., Ythier, A., Moine, P., Robert, N., Jourdan, E., Rossi, J. F. & Klein, B. APRIL and TACI interact with syndecan-1 on the surface of multiple myeloma cells to form an essential survival loop. Eur J Haematol 83, 119-129, (2009).
21 O'Connell, F. P., Pinkus, J. L. & Pinkus, G. S. CD138 (syndecan-1), a plasma cell marker immunohistochemical profile in hematopoietic and nonhematopoietic neoplasms. Am J Clin Pathol 121, 254-263, (2004).
22 Fujino, M. The histopathology of myeloma in the bone marrow. J Clin Exp Hematop 58, 61-67, (2018).
23 Lamorte, S., Ferrero, S., Aschero, S., Monitillo, L., Bussolati, B., Omede, P., Ladetto, M. & Camussi, G. Syndecan-1 promotes the angiogenic phenotype of multiple myeloma endothelial cells. Leukemia 26, 1081-1090, (2012).
24 Lin, P., Owens, R., Tricot, G. & Wilson, C. S. Flow Cytometric Immunophenotypic Analysis of 306 Cases of Multiple Myeloma. Am J Clin Pathol 121, 482-488, (2004).
25 Vasuthasawat, A., Yoo, E. M., Trinh, K. R., Lichtenstein, A., Timmerman, J. M. & Morrison, S. L. Targeted immunotherapy using anti-CD138-interferon alpha fusion proteins and bortezomib results in synergistic protection against multiple myeloma. MAbs 8, 1386-1397, (2016).
26 Yoo, E. M., Trinh, K. R., Tran, D., Vasuthasawat, A., Zhang, J., Hoang, B., Lichtenstein, A. & Morrison, S. L. Anti-CD138-targeted interferon is a potent therapeutic against multiple myeloma. J Interferon Cytokine Res 35, 281-291, (2015).
27 Sun, C., Mahendravada, A., Ballard, B., Kale, B., Ramos, C., West, J., Maguire, T., McKay, K., Lichtman, E., Tuchman, S., Dotti, G. & Savoldo, B. Safety and efficacy of targeting CD138 with a chimeric antigen receptor for the treatment of multiple myeloma. Oncotarget 10, 2369-2383, (2019).
28 Jamet, B., Bailly, C., Carlier, T., Touzeau, C., Nanni, C., Zamagni, E., Barre, L., Michaud, A. V., Cherel, M., Moreau, P., Bodet-Milin, C. & Kraeber-Bodere, F. Interest of Pet Imaging in Multiple Myeloma. Front Med (Lausanne) 6, 69, (2019).
29 Lendorf, M. E., Manon-Jensen, T., Kronqvist, P., Multhaupt, H. A. & Couchman, J. R. Syndecan-1 and syndecan-4 are independent indicators in breast carcinoma. J Histochem Cytochem 59, 615-629, (2011).
30 Nguyen, T. L., Grizzle, W. E., Zhang, K., Hameed, O., Siegal, G. P. & Wei, S. Syndecan-1 overexpression is associated with nonluminal subtypes and poor prognosis in advanced breast cancer. Am J Clin Pathol 140, 468-474, (2013).
31 Schonfeld, K., Herbener, P., Zuber, C., Hader, T., Bemoster, K., Uherek, C. & Schuttrumpf, J. Activity of Indatuximab Ravtansine against Triple-Negative Breast Cancer in Preclinical Tumor Models. Pharm Res 35, 118, (2018).
32 Szatmari, T., Mundt, F., Kumar-Singh, A., Mobus, L., Otvos, R., Hjerpe, A. & Dobra, K. Molecular targets and signaling pathways regulated by nuclear translocation of syndecan-1. BMC Cell Biol 18, 34, (2017).
33 Szatmari, T., Otvos, R., Hjerpe, A. & Dobra, K. Syndecan-1 in Cancer: Implications for Cell Signaling, Differentiation, and Prognostication. Dis Markers 2015, 796052, (2015).
34 van de Donk, N., Richardson, P. G. & Malavasi, F. CD38 antibodies in multiple myeloma: back to the future. Blood 131, 13-29, (2018).
35 van de Donk, N. W., Janmaat, M. L., Mutis, T., Lammerts van Bueren, J. J., Ahmadi, T., Sasser, A. K., Lokhorst, H. M. & Parren, P. W. Monoclonal antibodies targeting CD38 in hematological malignancies and beyond. Immunol Rev 270, 95-112, (2016).
36 Naik, J., Themeli, M., de Jong-Korlaar, R., Ruiter, R. W. J., Poddighe, P. J., Yuan, H., de Bruijn, J. D., Ossenkoppele, G. J., Zweegman, S., Smit, L., Mutis, T., Martens, A. C. M., van de Donk, N. & Groen, R. W. J. CD38 as a therapeutic target for adult acute myeloid leukemia and T-cell acute lymphoblastic leukemia. Haematologica 104, e100-e103, (2019).
37 Marinov, J., Koubek, K. & Stary, J. Immunophenotypic significance of the “lymphoid” CD38 antigen in myeloid blood malignancies. Neoplasma 40, 355-358, (1993).
38 Suzuki, R., Suzumiya, J., Nakamura, S., Aoki, S., Notoya, A., Ozaki, S., Gondo, H., Hino, N., Mori, H., Sugimori, H., Kawa, K., Oshimi, K. & Group, N. K.-c. T. S. Aggressive natural killer-cell leukemia revisited: large granular lymphocyte leukemia of cytotoxic NK cells. Leukemia 18, 763-770, (2004).
39 Wang, L., Wang, H., Li, P. F., Lu, Y., Xia, Z. J., Huang, H. Q. & Zhang, Y. J. CD38 expression predicts poor prognosis and might be a potential therapy target in extranodal NK/T cell lymphoma, nasal type. Ann Hematol 94, 1381-1388, (2015).
40 Parry-Jones, N., Matutes, E., Morilla, R., Brito-Babapulle, V., Wotherspoon, A., Swansbury, G. J. & Catovsky, D. Cytogenetic abnormalities additional to t(11;14) correlate with clinical features in leukaemic presentation of mantle cell lymphoma, and may influence prognosis: a study of 60 cases by FISH. Br J Haematol 137, 117-124, (2007).
41 Morandi, F., Horenstein, A. L., Costa, F., Giuliani, N., Pistoia, V. & Malavasi, F. CD38: A Target for Immunotherapeutic Approaches in Multiple Myeloma. Front Immunol 9, 2722, (2018).
42 Guang, M. H. Z., McCann, A., Bianchi, G., Zhang, L., Dowling, P., Bazou, D., O'Gorman, P. & Anderson, K. C. Overcoming multiple myeloma drug resistance in the era of cancer ‘omics’. Leuk Lymphoma 59, 542-561, (2018).
43 Kumar, S. K., Rajkumar, V., Kyle, R. A., van Duin, M., Sonneveld, P., Mateos, M. V., Gay, F. & Anderson, K. C. Multiple myeloma. Nat Rev Dis Primers 3, 17046, (2017).
44 Rajkumar, S. V. Multiple myeloma: Every year a new standard? Hematol Oncol 37 Suppl 1, 62-65, (2019).
45 Rhodes, D. R., Kalyana-Sundaram, S., Mahavisno, V., Varambally, R., Yu, J., Briggs, B. B., Barrette, T. R., Anstet, M. J., Kincead-Beal, C., Kulkarni, P., Varambally, S., Ghosh, D. & Chinnaiyan, A. M. Oncomine 3.0: genes, pathways, and networks in a collection of 18,000 cancer gene expression profiles. Neoplasia (New York, N.Y.) 9, 166-180, (2007).
46 Uhlén, M., Fagerberg, L., Hallstróm, B. M., Lindskog, C., Oksvold, P., Mardinoglu, A., Sivertsson, A., Kampf, C., Sjostedt, E., Asplund, A., Olsson, I., Edlund, K., Lundberg, E., Navani, S., Szigyarto, C. A.-K., Odeberg, J., Djureinovic, D., Takanen, J. O., Hober, S., Alm, T., Edqvist, P.-H., Berling, H., Tegel, H., Mulder, J., Rockberg, J., Nilsson, P., Schwenk, J. M., Hamsten, M., von Feilitzen, K., Forsberg, M., Persson, L., Johansson, F. et al. Proteomics. Tissue-based map of the human proteome. Science (New York, N.Y.) 347, 1260419-1260419, (2015).
47 Daniels, T. R., Delgado, T., Rodriguez, J. A., Helguera, G. & Penichet, M. L. The transferrin receptor part I: Biology and targeting with cytotoxic antibodies for the treatment of cancer. Clin Immunol 121, 144-158, (2006).
48 Shen, Y., Li, X., Dong, D., Zhang, B., Xue, Y. & Shang, P. Transferrin receptor 1 in cancer: a new sight for cancer therapy. Am J Cancer Res 8, 916-931, (2018).
49 Nagai, K., Nakahata, S., Shimosaki, S., Tamura, T., Kondo, Y., Baba, T., Taki, T., Taniwaki, M., Kurosawa, G., Sudo, Y., Okada, S., Sakoda, S. & Morishita, K. Development of a complete human anti-human transferrin receptor C antibody as a novel marker of oral dysplasia and oral cancer. Cancer Med 3, 1085-1099, (2014).
50 Pizzamiglio, S., De Bortoli, M., Taverna, E., Signore, M., Veneroni, S., Cho, W. C.-S., Orlandi, R., Verderio, P. & Bongarzone, I. Expression of Iron-Related Proteins Differentiate Non-Cancerous and Cancerous Breast Tumors. Int J Mol Sci 18, 410-410, (2017).
51 Rosager, A. M., Sorensen, M. D., Dahlrot, R. H., Hansen, S., Schonberg, D. L., Rich, J. N., Lathia, J. D. & Kristensen, B. W. Transferrin receptor-1 and ferritin heavy and light chains in astrocytic brain tumors: Expression and prognostic value. PLoS One 12, e0182954, (2017).
52 Parenti, R., Salvatorelli, L. & Magro, G. Anaplastic Thyroid Carcinoma: Current Treatments and Potential New Therapeutic Options with Emphasis on TfR1/CD71. Int J Endocrinol 2014, 685396, (2014).
53 Campisi, A., Bonfanti, R., Raciti, G., Bonaventura, G., Legnani, L., Magro, G., Pennisi, M., Russo, G., Chiacchio, M. A., Pappalardo, F. & Parenti, R. Gene Silencing of Transferrin-1 Receptor as a Potential Therapeutic Target for Human Follicular and Anaplastic Thyroid Cancer. Mol Ther Oncolytics 16, 197-206, (2020).
54 Sakurai, K., Sohda, T., Ueda, S., Tanaka, T., Hirano, G., Yokoyama, K., Morihara, D., Aanan, A., Takeyama, Y., Irie, M., Iwata, K., Syakado, S., Noritomiz, T., Yamashita, Y. & Sakisaka, S. Immunohistochemical demonstration of transferrin receptor 1 and 2 in human hepatocellular carcinoma tissue. Hepatogastroenterology 61, 426-430, (2014).
55 Boult, J., Roberts, K., Brookes, M. J., Hughes, S., Bury, J. P., Cross, S. S., Anderson, G. J., Spychal, R., Iqbal, T. & Tselepis, C. Overexpression of cellular iron import proteins is associated with malignant progression of esophageal adenocarcinoma. Clin Cancer Res 14, 379-387, (2008).
56 Xue, X., Ramakrishnan, S. K., Weisz, K., Triner, D., Xie, L., Attili, D., Pant, A., Gyorffy, B., Zhan, M., Carter-Su, C., Hardiman, K. M., Wang, T. D., Dame, M. K., Varani, J., Brenner, D., Fearon, E. R. & Shah, Y. M. Iron Uptake via DMT1 Integrates Cell Cycle with JAK-STAT3 Signaling to Promote Colorectal Tumorigenesis. Cell Metab 24, 447-461, (2016).
57 Fu, Y., Lin, L. & Xia, L. MiR-107 function as a tumor suppressor gene in colorectal cancer by targeting transferrin receptor 1. Cell Mol Biol Lett 24, 31, (2019).
58 Babu, K. R. & Muckenthaler, M. U. miR-148a regulates expression of the transferrin receptor 1 in hepatocellular carcinoma. Sci Rep 9, 1518, (2019).
59 Bahrami, S., Kazemi, B., Zali, H., Black, P. C., Basiri, A., Bandehpour, M., Hedayati, M. & Sahebkar, A. Discovering Therapeutic Protein Targets for Bladder Cancer Using Proteomic Data Analysis. Curr Mol Pharmacol, 13, 150-172, (2020).
60 Ye, J., Ma, J., Liu, C., Huang, J., Wang, L. & Zhong, X. A novel iron(II) phenanthroline complex exhibits anticancer activity against TFR1-overexpressing esophageal squamous cell carcinoma cells through ROS accumulation and DNA damage. Biochem Pharmacol 166, 93-107, (2019).
61 Cheng, X., Fan, K., Wang, L., Ying, X., Sanders, A. J., Guo, T., Xing, X., Zhou, M., Du, H., Hu, Y., Ding, H., Li, Z., Wen, X., Jiang, W., Yan, X. & Ji, J. TfR1 binding with H-ferritin nanocarrier achieves prognostic diagnosis and enhances the therapeutic efficacy in clinical gastric cancer. Cell Death Dis 11, 92, (2020).
62 Gu, Z., Wang, H., Xia, J., Yang, Y., Jin, Z., Xu, H., Shi, J., De Domenico, I., Tricot, G. & Zhan, F. Decreased ferroportin promotes myeloma cell growth and osteoclast differentiation. Cancer Res 75, 2211-2221, (2015).
63 Essaghir, A. & Demoulin, J. B. A minimal connected network of transcription factors regulated in human tumors and its application to the quest for universal cancer biomarkers. PLoS One 7, e39666, (2012).
64 Chan, K. T., Choi, M. Y., Lai, K. K. Y., Tan, W., Tung, L. N., Lam, H. O. Y. U. H. Y., Tong, D. K. H., Lee, N. P. & Law, S. Overexpression of transferrin receptor CD71 and its tumorigenic properties in esophageal squamous cell carcinoma. Oncol Rep 31, 1296-1304, (2014).
65 Habashy, H. O., Powe, D. G., Staka, C. M., Rakha, E. A., Ball, G., Green, A. R., Aleskandarany, M., Paish, E. C., Douglas MacMillan, R., Nicholson, R. I., Ellis, I. O. & Gee, J. M. W. Transferrin receptor (CD71) is a marker of poor prognosis in breast cancer and can predict response to tamoxifen. Breast Cancer Res Treat 119, 283-293, (2010).
66 Ding Cheng, Y., Wang, F., Elliott, R. L. & Head, J. F. Expression of transferrin receptor and ferritin H-chain mRNA are associated with clinical and histopathological prognostic indicators in breast cancer. Anticancer Res 21, 541-549, (2001).
67 Basuli, D., Tesfay, L., Deng, Z., Paul, B., Yamamoto, Y., Ning, G., Xian, W., McKeon, F., Lynch, M., Crum, C. P., Hegde, P., Brewer, M., Wang, X., Miller, L. D., Dyment, N., Torti, F. M. & Torti, S. V. Iron addiction: a novel therapeutic target in ovarian cancer. Oncogene 36, 4089-4099, (2017).
68 Kondo, K., Noguchi, M., Mukai, K., Matsuno, Y., Sato, Y., Shimosato, Y. & Monden, Y. Transferrin receptor expression in adenocarcinoma of the lung as a histopathologic indicator of prognosis. Chest 97, 1367-1371, (1990).
69 Xu, X., Liu, T., Wu, J., Wang, Y., Hong, Y. & Zhou, H. Transferrin receptor-involved HIF-1 signaling pathway in cervical cancer. Cancer Gene Ther 26, 356-365, (2019).
70 Smith, N. W., Strutton, G. M., Walsh, M. D., Wright, G. R., Seymour, G. J., Lavin, M. F. & Gardiner, R. A. Transferrin receptor expression in primary superficial human bladder tumours identifies patients who develop recurrences. Br J Urol 65, 339-344, (1990).
71 Wu, H., Zhang, J., Dai, R., Xu, J. & Feng, H. Transferrin receptor-1 and VEGF are prognostic factors for osteosarcoma. J Orthop Surg Res 14, 296, (2019).
72 Ryschich, E., Huszty, G., Knaebel, H. P., Hartel, M., Buchler, M. W. & Schmidt, J. Transferrin receptor is a marker of malignant phenotype in human pancreatic cancer and in neuroendocrine carcinoma of the pancreas. Eur J Cancer 40, 1418-1422, (2004).
73 Jamnongkan, W., Thanan, R., Techasen, A., Namwat, N., Loilome, W., Intarawichian, P., Titapun, A. & Yongvanit, P. Upregulation of transferrin receptor-1 induces cholangiocarcinoma progression via induction of labile iron pool. Tumour Biol 39, 1010428317717655, (2017).
74 Greene, C. J., Attwood, K., Sharma, N. J., Gross, K. W., Smith, G. J., Xu, B. & Kauffman, E. C. Transferrin receptor 1 upregulation in primary tumor and downregulation in benign kidney is associated with progression and mortality in renal cell carcinoma patients. Oncotarget 8, 107052-107075, (2017).
75 Moon, S. J., Kim, J. H., Kong, S. H. & Shin, C. S. Protein Expression of Cyclin B1, Transferrin Receptor, and Fibronectin Is Correlated with the Prognosis of Adrenal Cortical Carcinoma. Endocrinol Metab (Seoul) 35, 132-141, (2020).
76 Prior, R., Reifenberger, G. & Wechsler, W. Transferrin receptor expression in tumours of the human nervous system: relation to tumour type, grading and tumour growth fraction. Virchows Archiv. A, Pathological anatomy and histopathology 416, 491-496, (1990).
77 Das Gupta, A., Patil, J. & Shah, V. I. Transferrin receptor expression by blast cells in acute lymphoblastic leukemia correlates with white cell count & amp; immunophenotype. Indian J Med Res 104, 226-233, (1996).
78 Das Gupta, A. & Shah, V. I. Correlation of transferrin receptor expression with histologic grade and immunophenotype in chronic lymphocytic leukemia and non-Hodgkin's lymphoma. Hematol Pathol 4, 37-41, (1990).
79 Hagag, A. A., Badraia, I. M., Abdelmageed, M. M., Hablas, N. M., Hazzaa, S. M. E. & Nosair, N. A. Prognostic Value of Transferrin Receptor-1 (CD71) Expression in Acute Lymphoblastic Leukemia. Endocr Metab Immune DisordD rug Targets 18, 610-617, (2018).
80 Habeshaw, J. A., Lister, T. A., Stansfeld, A. G. & Greaves, M. F. Correlation of transferrin receptor expression with histological class and outcome in non-hodgkin lymphoma. The Lancet 321, 498-501, (1983).
81 Maguire, A., Chen, X., Wisner, L., Malasi, S., Ramsower, C., Kendrick, S., Barrett, M. T., Glinsmann-Gibson, B., McGrath, M. & L., R. Potential Alternative Survival Mechanisms in HIV-Associated Diffuse Large B-Cell Lymphoma (DLBCL) of Germinal Center (GCB) Origin. International Confereence on Malignancies in HIV/AIDS (Oct. 21-22, 2019; Bethesda, Maryland, USA). Abstract No. O10.
82 Maguire, A., Chen, X., Wisner, L., Ramsower, C., Glinsmann-Gibson, B. & Rimsza, L. M. Over-Expression of Transferrin Receptor (TFRC/CD71) and Low Expression of Innate and Adaptive Immune Cell Subsets in HIV-Associated, GCB-DLBCL By Digital Gene Expression Profiling. The American Society of Hematology Annual Meeting (Nov. 13, 2019; Orlando, Florida, USA). Blood (2019), 134 (Supplement_1): 2783.
83 Ni, X. R., Zhao, Y. Y., Cai, H. P., Yu, Z. H., Wang, J., Chen, F. R., Yu, Y. J., Feng, G. K. & Chen, Z. P. Transferrin receptor 1 targeted optical imaging for identifying glioma margin in mouse models. J Neurooncol 148, 245-258, (2020).
84 Preithner, S., Elm, S., Lippold, S., Locher, M., Wolf, A., da Silva, A. J., Baeuerle, P. A. & Prang, N. S. High concentrations of therapeutic IgG1 antibodies are needed to compensate for inhibition of antibody-dependent cellular cytotoxicity by excess endogenous immunoglobulin G. Mol Immunol 43, 1183-1193, (2006).
85 Clynes, R. A., Towers, T. L., Presta, L. G. & Ravetch, J. V. Inhibitory Fc receptors modulate in vivo cytoxicity against tumor targets. Nat Med 6, 443-446, (2000).
86 Redman, J. M., Hill, E. M., AlDeghaither, D. & Weiner, L. M. Mechanisms of action of therapeutic antibodies for cancer. Mol Immunol 67, 28-45, (2015).
87 Nimmerjahn, F. & Ravetch, J. V. Antibodies, Fc receptors and cancer. Curr Opin Immunol 19, 239-245, (2007).
88 Devetzoglou, M., Vyzoukaki, R., Kokonozaki, M., Xekalou, A., Pappa, C. A., Papadopoulou, A., Alegakis, A., Androulakis, N. & Alexandrakis, M. G. High density of tryptase-positive mast cells in patients with multiple myeloma: correlation with parameters of disease activity. Tumour Biol 36, 8491-8497, (2015).
89 Ribatti, D., Moschetta, M. & Vacca, A. Macrophages in multiple myeloma. Immunol Lett 161, 241-244, (2014).
90 Zheng, Y., Cai, Z., Wang, S., Zhang, X., Qian, J., Hong, S., Li, H., Wang, M., Yang, J. & Yi, Q. Macrophages are an abundant component of myeloma microenvironment and protect myeloma cells from chemotherapy drug-induced apoptosis. Blood 114, 3625-3628, (2009).
91 Wasiuk, A., de Vries, V. C., Nowak, E. C. & Noelle, R. J. Mast Cells in Allergy and Tumor Disease. In “Cancer and IgE: Introducing the Concept of AllergoOncology”. Penichet M. L. and Jensen-Jarolim E., eds. Springer, New York, USA. 2010, pp. 137-158.
92 Josephs, D. H., Bax, H. J., Dodev, T., Georgouli, M., Nakamura, M., Pellizzari, G., Saul, L., Karagiannis, P., Cheung, A., Herraiz, C., Ilieva, K. M., Correa, I., Fittall, M., Crescioli, S., Gazinska, P., Woodman, N., Mele, S., Chiaruttini, G., Gilbert, A. E., Koers, A., Bracher, M., Selkirk, C., Lentfer, H., Barton, C., Lever, E., Muirhead, G., Tsoka, S., Canevari, S., Figini, M., Montes, A., Downes, N., Dombrowicz, D., Corrigan, C. J., Beavil, A. J., Nestle, F. O., Jones, P. S., Gould, H. J., Sanz-Moreno, V., Blower, P. J., Spicer, J. F. & Karagiannis, S. N. Anti-Folate Receptor-alpha IgE but not IgG Recruits Macrophages to Attack Tumors via TNFalpha/MCP-1 Signaling. Cancer Res 77, 1127-1141, (2017).
93 Karagiannis, S. N., Josephs, D. H., Bax, H. J. & Spicer, J. F. Therapeutic IgE Antibodies: Harnessing a Macrophage-Mediated Immune Surveillance Mechanism against Cancer. Cancer Res 77, 2779-2783, (2017).
94 Dalton, D. K. & Noelle, R. J. The roles of mast cells in anticancer immunity. Cancer Immunol Immunother 61, 1511-1520, (2012).
95 Teo, P. Z., Utz, P. J. & Mollick, J. A. Using the allergic immune system to target cancer: activity of IgE antibodies specific for human CD20 and MUC1. Cancer Immunol Immunother 61, 2295-2309, (2012).
96 Plotkin, J. D., Elias, M. G., Fereydouni, M., Daniels-Wells, T. R., Dellinger, A. L., Penichet, M. L. & Kepley, C. L. Human Mast Cells From Adipose Tissue Target and Induce Apoptosis of Breast Cancer Cells. Front Immunol 10, 138, (2019).
97 Karagiannis, S. N., Bracher, M. G., Beavil, R. L., Beavil, A. J., Hunt, J., McCloskey, N., Thompson, R. G., East, N., Burke, F., Sutton, B. J., Dombrowicz, D., Balkwill, F. R. & Gould, H. J. Role of IgE receptors in IgE antibody-dependent cytotoxicity and phagocytosis of ovarian tumor cells by human monocytic cells. Cancer Immunol Immunother 57, 247-263, (2008).
98 Karagiannis, S. N., Bracher, M. G., Hunt, J., McCloskey, N., Beavil, R. L., Beavil, A. J., Fear, D. J., Thompson, R. G., East, N., Burke, F., Moore, R. J., Dombrowicz, D. D., Balkwill, F. R. & Gould, H. J. IgE-antibody-dependent immunotherapy of solid tumors: cytotoxic and phagocytic mechanisms of eradication of ovarian cancer cells. J. Immunol. 179, 2832-2843, (2007).
99 Pellizzari, G., Hoskin, C., Crescioli, S., Mele, S., Gotovina, J., Chiaruttini, G., Bianchini, R., Ilieva, K., Bax, H. J., Papa, S., Lacy, K. E., Jensen-Jarolim, E., Tsoka, S., Josephs, D. H., Spicer, J. F. & Karagiannis, S. N. IgE re-programs alternatively-activated human macrophages towards pro-inflammatory anti-tumoural states. EBioMedicine 43, 67-81, (2019).
100 Engeroff, P., Fellmann, M., Yerly, D., Bachmann, M. F. & Vogel, M. A novel recycling mechanism of native IgE-antigen complexes in human B cells facilitates transfer of antigen to dendritic cells for antigen presentation. J Allergy Clin Immunol 142, 557-568 e556, (2018).
101 Nigro, E. A., Brini, A. T., Soprana, E., Ambrosi, A., Dombrowicz, D., Siccardi, A. G. & Vangelista, L. Antitumor IgE adjuvanticity: key role of Fc epsilon RI. J Immunol 183, 4530-4536, (2009).
102 Daniels-Wells, T. R., Helguera, G., Leuchter, R. K., Quintero, R., Kozman, M., Rodriguez, J. A., Ortiz-Sanchez, E., Martinez-Maza, O., Schultes, B. C., Nicodemus, C. F. & Penichet, M. L. A novel IgE antibody targeting the prostate-specific antigen as a potential prostate cancer therapy. BMC Cancer 13, 195, (2013).
103 Daniels, T. R., Leuchter, R. K., Quintero, R., Helguera, G., Rodriguez, J. A., Martinez-Maza, O., Schultes, B. C., Nicodemus, C. F. & Penichet, M. L. Targeting HER2/neu with a fully human IgE to harness the allergic reaction against cancer cells. Cancer Immunol Immunother 61, 991-1003, (2012).
104 Platzer, B., Dehlink, E., Turley, S. J. & Fiebiger, E. How to connect an IgE-driven response with CTL activity? Cancer Immunol Immunother 61, 1521-1525, (2012).
105 Nigro, E. A., Siccardi, A. G. & Vangelista, L. IgE as Adjuvant in Tumor Vaccination. In “Cancer and IgE: Introducing the Concept of AllergoOncology”. Penichet M. L. and Jensen-Jarolim E., eds. Springer, New York, USA. 2010, pp. 215-230.
106 Nigro, E. A., Soprana, E., Brini, A. T., Ambrosi, A., Yenagi, V. A., Dombrowicz, D., Siccardi, A. G. & Vangelista, L. An antitumor cellular vaccine based on a mini-membrane IgE. J Immunol 188, 103-110, (2012).
107 de Vries, V. C., Wasiuk, A., Bennett, K. A., Benson, M. J., Elgueta, R., Waldschmidt, T. J. & Noelle, R. J. Mast cell degranulation breaks peripheral tolerance Am J Transplant 9, 2270-2280, (2009).
108 Doshi, P. Combination therapies with anti-CD38 antibodies. United States Patent Application No. US 2015/024.6123 A1. Pub. Date: Sep. 3, 2015.
109 Kraus, E., Bruecher, C., Daelken, B., Germer, M., Zeng, S., Osterroth, F., Uherek, C. & Aigner, S. Agents targeting CD138 and uses thereof. United States Patent Application No. US Patent 2009/0175863 A1. Pub. Date: Dec. 29, 2015.
110 Ng, P. P., Helguera, G., Daniels, T. R., Lomas, S. Z., Rodriguez, J. A., Schiller, G., Bonavida, B., Morrison, S. L. & Penichet, M. L. Molecular events contributing to cell death in malignant human hematopoietic cells elicited by an IgG3-avidin fusion protein targeting the transferrin receptor. Blood 108, 2745-2754, (2006).
111 Daniels-Wells, T. R., Candelaria, P. V., Leoh, L. S., Nava, M., Martinez-Maza, O. & Penichet, M. L. An IgG1 Version of the Anti-transferrin Receptor 1 Antibody ch128.1 Shows Significant Antitumor Activity Against Different Xenograft Models of Multiple Myeloma: A Brief Communication. J Immunother 43, 48-52, (2020).
112 Penichet, M. L., Wells, T. R., Candelaria, P. V. & Almagro, J. C. Compositions and methods for transferrin receptor 1 targeting. International Application No. PCT/US2020/059532. Pub. Date: May 14, 2021.
113 Wiegand, T. W., Williams, P. B., Dreskin, S. C., Jouvin, M. H., Kinet, J. P. & Tasset, D. High-affinity oligonucleotide ligands to human IgE inhibit binding to Fc epsilon receptor I. J Immunol 157, 221-230, (1996).
114 Lyczak, J. B., Zhang, K., Saxon, A. & Morrison, S. L. Expression of novel secreted isoforms of human immunoglobulin E proteins. J Biol Chem 271, 3428-3436, (1996).
115 Daniels, T. R., Martinez-Maza, O. & Penichet, M. L. Animal models for IgE-meditated cancer immunotherapy. Cancer Immunol Immunother 61, 1535-1546, (2012).
116 Bracher, M., Gould, H. J., Sutton, B. J., Dombrowicz, D. & Karagiannis, S. N. Three-colour flow cytometric method to measure antibody-dependent tumour cell killing by cytotoxicity and phagocytosis. J Immunol Methods 323, 160-171, (2007).
117 Leoh, L. S., Kim, Y. K., Candelaria, P. V., Martinez-Maza, O., Daniels-Wells, T. R. & Penichet, M. L. Efficacy and Mechanism of Antitumor Activity of an Antibody Targeting Transferrin Receptor 1 in Mouse Models of Human Multiple Myeloma. J Immunol 200, 3485-3494, (2018).

What is claimed:

1. An antibody comprising:
(a) a V_{H} region of a CD38-binding antibody;
(b) a V_{L} region of the CD38-binding antibody; and
(c) a C_{H} region of an immunoglobulin epsilon heavy chain.

2. The antibody of claim 1, wherein the V_{H} region comprises SEQ ID NO: 11, SEQ ID NO:12, and SEQ ID NO:13.

3. The antibody of claim 1 or 2, wherein the V_{L} region comprises SEQ ID NO:14, SEQ ID NO:15, and SEQ ID NO:16.

4. The antibody of any of claims 1-3, wherein the CD38-binding antibody is isatuximab, felzartamab, daratumumab, or a Fab fragment or single chain variable fragment (scFv) thereof.

5. The antibody of any of claims 1-4, wherein the CD38-binding antibody is daratumumab or a Fab fragment or scFv thereof.

6. The antibody of any of claims 1-5, wherein the V_{H} region has at least 95% sequence identity with SEQ ID NO:17.

7. The antibody of claim 6, wherein the V_{H} region comprises SEQ ID NO:17.

8. The antibody of any of claims 1-7, wherein the V_{L} region has at least 95% sequence identity with SEQ ID NO:18.

9. The antibody of claim 8, wherein the V_{L} region comprises SEQ ID NO:18.

10. The antibody of any of claims 1-9, wherein the C_{H} region comprises SEQ ID NO:9.

11. The antibody of any of claims 1-10, further comprising a light chain constant (C_{L}) region.

12. The antibody of claim 11, wherein the C_{L} region is a C_{L} region of an immunoglobulin kappa constant chain.

13. The antibody of claim 11 or 12, wherein the C_{L} region comprises SEQ ID NO:10.

14. The antibody of any of claims 11-13, wherein the C_{L} region is a C_{L} region of an immunoglobulin lambda constant chain.

15. The antibody of any of claims 1-14, wherein the antibody comprises a sequence having at least 95% identity to SEQ ID NO:29.

16. The antibody of claim 15, wherein the antibody comprises SEQ ID NO:29.

17. The antibody of any of claims 1-16, wherein the antibody comprises a sequence having at least 95% identity to SEQ ID NO:30.

18. The antibody of claim 17, wherein the antibody comprises SEQ ID NO:30.

19. A nucleic acid comprising a nucleotide sequence encoding for the antibody of any of claims 1-18.

20. A vector comprising the nucleic acid of claim 19.

21. A pharmaceutical composition comprising:
(i) the antibody of any of claims 1-18;
(ii) the nucleic acid of claim 19; or
(iii) the vector of claim 20;
and a pharmaceutically acceptable excipient.

22. The pharmaceutical composition of claim 21, further comprising an additional therapeutic.

23. The pharmaceutical composition of claim 22, wherein the additional therapeutic is a chemotherapeutic, a nucleic acid, a protein, or a nanodrug.

24. The pharmaceutical composition of claim 22, wherein the nucleic acid is an antisense oligonucleotide, a small interfering RNA (siRNA), a clustered regularly interspaced short palindromic repeats (CRISPR)-based gene therapy, or a viral vector.

25. The pharmaceutical composition of claim 22, wherein the additional therapeutic is a protein, wherein the protein is a toxin or an enzyme.

26. The pharmaceutical composition of any of claims 22-25, wherein the additional therapeutic is operatively linked to the antibody.

27. A composition comprising the antibody of any of claims 1-18 bound to a CD38 molecule.

28. The composition of claim 27, wherein the CD38 molecule is a soluble CD38 molecule.

29. The composition of claim 27, wherein the CD38 molecule is attached to the surface of a cancer cell.

30. The composition of claim 29, wherein the cancer cell was previously irradiated.

31. The composition of any of claims 27-30, wherein the antibody is further bound to an antigen-presenting cell via the Fc domain.

32. The composition of claim 31, wherein the antigen-presenting cell is a dendritic cell.

33. The composition of any of claims 27-32, further comprising an adjuvant.

34. A method for treating a subject for cancer, the method comprising administering to the subject an effective amount of (I) the antibody of any of claims 1-18, (II) the nucleic acid of claim 19, (III) the vector of claim 20, (IV) the pharmaceutical composition of any of claims 21-26, or (V) the composition of any of claims 27-32.

35. The method of claim 34, wherein the cancer is esophageal squamous cell carcinoma, breast cancer, ovarian cancer, lung cancer, cervical cancer, bladder cancer, osteosarcoma, pancreatic cancers, cholangiocarcinoma, renal cell carcinoma, hepatocellular carcinoma, adrenal cortical carcinoma, glioblastoma, acute lymphoblastic leukemia (ALL), acute myeloid leukemia (AML), chronic lymphocytic leukemia (CLL), aggressive natural killer (NK) cell leukemia (ANKL), or a non-Hodgkin lymphoma (NHL) including NK/T-cell lymphoma and mantle cell lymphoma (MCL).

36. The method of claim 34, wherein the cancer is multiple myeloma (MM).

37. The method of claim 34 or 36, further comprising administering an additional cancer therapy to the subject.

38. The method of claim 37, wherein the additional cancer therapy is radiotherapy, chemotherapy, or immunotherapy.

39. A method for preventing cancer, the method comprising administering to a subject an effective amount of (I) the antibody of any of claims 1-18, (II) the nucleic acid of claim 19, (III) the vector of claim 20, (IV) the pharmaceutical composition of any of claims 21-26, or (V) the composition of any of claims 27-32.

40. The method of claim 39, wherein the cancer is esophageal squamous cell carcinoma, breast cancer, ovarian cancer, lung cancer, cervical cancer, bladder cancer, osteosarcoma, pancreatic cancers, cholangiocarcinoma, renal cell carcinoma, hepatocellular carcinoma, adrenal cortical carcinoma, glioblastoma, AMK, ANKL, ALL, CLL, or NHL

41. The method of claim 39, wherein the cancer is MM.

42. A method of diagnosing a subject for cancer comprising providing to the subject the antibody of any of claims 1-18 and a diagnostic agent.

43. The method of claim 42, wherein the diagnostic agent is a dye.

44. A kit comprising (i) the antibody of any of claims 1-18 and (ii) instructions for use for detecting CD38 in a biological sample.

45. An antibody comprising:
(a) a heavy chain variable (V_{H}) region of a CD138-binding antibody;
(b) a light chain variable (V_{L}) region of the CD138-binding antibody; and
(c) a heavy chain constant (C_{H}) region of an immunoglobulin epsilon heavy chain.

46. The antibody of claim 45, wherein the CD138-binding antibody is B-B4, BC/B-B4, B-B2, DL-101, 1D4, M115, 1.BB.210, 2Q1484, 5F7, 104-9, 281-2, or a Fab fragment or scFv thereof.

47. The antibody of claim 46, wherein the CD138-binding antibody is B-B4, 1D4, M115, or a Fab fragment or scFv thereof.

48. The antibody of any of claims 45-47, wherein the V_{H} region comprises SEQ ID NO:1, SEQ ID NO:2, and SEQ ID NO:3.

49. The antibody of any of claims 45-48, wherein the V_{L} region comprises SEQ ID NO:4, SEQ ID NO:5, and SEQ ID NO:6.

50. The antibody of any of claims 45-49, wherein the CD138-binding antibody is B-B4 or a Fab fragment or scFv thereof.

51. The antibody of any of claims 45-50, wherein the V_{H} region has at least 95% sequence identity with SEQ ID NO:7.

52. The antibody of claim 51, wherein the V_{H} region comprises SEQ ID NO:7.

53. The antibody of any of claims 45-52, wherein the V_{L} region has at least 95% sequence identity with SEQ ID NO:8.

54. The antibody of claim 53, wherein the V_{L} region comprises SEQ ID NO:8.

55. The antibody of any of claims 45-54, wherein the C_{H} region comprises SEQ ID NO:9.

56. The antibody of any of claims 45-55, further comprising a light chain constant (C_{L}) region.

57. The antibody of claim 56, wherein the C_{L} region is a C_{L} region of an immunoglobulin kappa constant chain.

58. The antibody of claim 56 or 57, wherein the C_{L} region comprises SEQ ID NO:10.

59. The antibody of any of claims 56-58, wherein the C_{L} region is a C_{L} region of an immunoglobulin lambda constant chain.

60. The antibody of any of claims 45-59, wherein the antibody comprises a sequence having at least 95% identity to SEQ ID NO:27.

61. The antibody of claim 60, wherein the antibody comprises SEQ ID NO:27.

62. The antibody of any of claims 45-61, wherein the antibody comprises a sequence having at least 95% identity to SEQ ID NO:28.

63. The antibody of claim 62, wherein the antibody comprises SEQ ID NO:28.

64. A nucleic acid comprising a nucleotide sequence encoding for the antibody of any of claims 45-63.

65. A vector comprising the nucleic acid of claim 64.

66. A pharmaceutical composition comprising:
(i) the antibody of any of claims 45-63;
(ii) the nucleic acid of claim 64; or
(iii) the vector of claim 65;
and a pharmaceutically acceptable excipient.

67. The pharmaceutical composition of claim 66, further comprising an additional therapeutic.

68. The pharmaceutical composition of claim 67, wherein the additional therapeutic is a chemotherapeutic, a nucleic acid, a protein, or a nanodrug.

69. The pharmaceutical composition of claim 68, wherein the nucleic acid is an antisense oligonucleotide, a small interfering RNA (siRNA), a clustered regularly interspaced short palindromic repeats (CRISPR)-based gene therapy, or a viral vector.

70. The pharmaceutical composition of claim 68, wherein the additional therapeutic is a protein, wherein the protein is a toxin or an enzyme.

71. The pharmaceutical composition of any of claims 67-70, wherein the additional therapeutic is operatively linked to the antibody.

72. A composition comprising the antibody of any of claims 45-63 bound to a CD138 molecule.

73. The composition of claim 72, wherein the CD138 molecule is a soluble CD138 molecule.

74. The composition of claim 72, wherein the CD138 molecule is attached to the surface of a cancer cell.

75. The composition of claim 74, wherein the cancer cell was previously irradiated.

76. The composition of any of claims 72-75, wherein the antibody is further bound to an antigen-presenting cell via the Fc domain.

77. The composition of claim 76, wherein the antigen-presenting cell is a dendritic cell.

78. The composition of any of claims 72-77, further comprising an adjuvant.

79. A method for treating a subject for cancer, the method comprising administering to the subject an effective amount of (I) the antibody of any of claims 45-63, (II) the nucleic acid of claim 64, (III) the vector of claim 65, (IV) the pharmaceutical composition of any of claims 66-71, or (V) the composition of any of claims 72-77.

80. The method of claim 79, wherein the cancer is esophageal squamous cell carcinoma, breast cancer, ovarian cancer, lung cancer, cervical cancer, bladder cancer, osteosarcoma, pancreatic cancers, cholangiocarcinoma, renal cell carcinoma, hepatocellular carcinoma, adrenal cortical carcinoma, glioblastoma, AMK, ANKL, ALL, CLL, or NHL.

81. The method of claim 79, wherein the cancer is breast cancer.

82. The method of claim 81, wherein the breast cancer is triple negative breast cancer.

83. The method of claim 79, wherein the cancer is MM.

84. The method of any of claims 79-83, further comprising administering an additional cancer therapy to the subject.

85. The method of claim 84, wherein the additional cancer therapy is radiotherapy, chemotherapy, or immunotherapy.

86. A method for preventing cancer, the method comprising administering to the subject an effective amount of (I) the antibody of any of claims 45-63, (II) the nucleic acid of claim 64, (III) the vector of claim 65, or (IV) the pharmaceutical composition of any of claims 66-71.

87. The method of claim 86, wherein the cancer is esophageal squamous cell carcinoma, breast cancer, ovarian cancer, lung cancer, cervical cancer, bladder cancer, osteosarcoma, pancreatic cancers, cholangiocarcinoma, renal cell carcinoma, hepatocellular carcinoma, adrenal cortical carcinoma, glioblastoma, AMK, ANKL, ALL, CLL, or NHL

88. The method of claim 86, wherein the cancer is breast cancer.

89. The method of claim 88, wherein the breast cancer is triple negative breast cancer.

90. The method of claim 86, wherein the cancer is MM.

91. The method of any of claims 86-90, wherein the method comprises eliminating pre-malignant cells from the subject.

92. The method of any of claims 86-91, wherein the method comprises inhibiting proliferation of pre-malignant cells from the subject.

93. A method of diagnosing a subject for cancer comprising providing to the subject the antibody of any of claims 45-63 and a diagnostic agent.

94. The method of claim 93, wherein the diagnostic agent is a dye.

95. A kit comprising (i) the antibody of any of claims 45-63 and (ii) instructions for use for detecting CD138 in a biological sample.

96. An antibody comprising:
(a) a V_{H} region of a TfR1-binding antibody;
(b) a V_{L} region of the TfR1-binding antibody; and
(c) a C_{H} region of an immunoglobulin epsilon heavy chain.

97. The antibody of claim 96, wherein the V_{H} region comprises SEQ ID NO:19, SEQ ID NO:20, and SEQ ID NO:21.

98. The antibody of claim 96 or 97, wherein the V_{L} region comprises SEQ ID NO:22, SEQ ID NO:23, and SEQ ID NO:24.

99. The antibody of any of claims 96-98, wherein the TfR1-binding antibody is 7579, E2.3, A27.15, B3/25, 43/31, D65.30, A24, RBC4, 42/6, D2C, JST-TFR09, H7, ch128.1, or a Fab fragment or scFv thereof.

100. The antibody of any of claims 96-99, wherein the TfR1-binding antibody is ch128.1 or a Fab fragment or scFv thereof.

101. The antibody of any of claims 96-100, wherein the V_{H} region has at least 95% sequence identity with SEQ ID NO:25.

102. The antibody of claim 101, wherein the V_{H} region comprises SEQ ID NO:25.

103. The antibody of any of claims 96-102, wherein the V_{L} region has at least 95% sequence identity with SEQ ID NO:26.

104. The antibody of claim 103, wherein the V_{L} region comprises SEQ ID NO:26.

105. The antibody of any of claims 96-104, wherein the C_{H} region comprises SEQ ID NO:9.

106. The antibody of any of claims 96-105, further comprising a light chain constant (C_{L}) region.

107. The antibody of claim 106, wherein the C_{L} region is a C_{L} region of an immunoglobulin kappa constant chain.

108. The antibody of claim 106 or 107, wherein the C_{L} region comprises SEQ ID NO:10.

109. The antibody of any of claims 106-108, wherein the C_{L} region is a C_{L} region of an immunoglobulin lambda constant chain.

110. The antibody of any of claims 96-109, wherein the antibody comprises a sequence having at least 95% identity to SEQ ID NO:31.

111. The antibody of claim 110, wherein the antibody comprises SEQ ID NO:31.

112. The antibody of any of claims 96-111, wherein the antibody comprises a sequence having at least 95% identity to SEQ ID NO:32.

113. The antibody of claim 112, wherein the antibody comprises SEQ ID NO:32.

114. A nucleic acid comprising a nucleotide sequence encoding for the antibody of any of claims 96-113.

115. A vector comprising the nucleic acid of claim 114.

116. A pharmaceutical composition comprising:
(i) the antibody of any of claims 96-113;
(ii) the nucleic acid of claim 114; or
(iii) the vector of claim 115;
and a pharmaceutically acceptable excipient.

117. The pharmaceutical composition of claim 116, further comprising an additional therapeutic.

118. The pharmaceutical composition of claim 117, wherein the additional therapeutic is a chemotherapeutic, a nucleic acid, a protein, or a nanodrug.

119. The pharmaceutical composition of claim 118, wherein the nucleic acid is an antisense oligonucleotide, a siRNA, a CRISPR-based gene therapy, or a viral vector.

120. The pharmaceutical composition of claim 118, wherein the additional therapeutic is a protein, wherein the protein is a toxin or an enzyme.

121. The pharmaceutical composition of any of claims 117-120, wherein the additional therapeutic is operatively linked to the antibody.

122. A composition comprising the antibody of any of claims 96-113 bound to a TfR1 molecule.

123. The composition of claim 122, wherein the TfR1 molecule is a soluble TfR1 molecule.

124. The composition of claim 122, wherein the TfR1 molecule is attached to the surface of a cancer cell.

125. The composition of claim 124, wherein the cancer cell was previously irradiated.

126. The composition of any of claims 122-125, wherein the antibody is further bound to an antigen-presenting cell via the Fc domain.

127. The composition of claim 126, wherein the antigen-presenting cell is a dendritic cell.

128. The composition of any of claims 122-127, further comprising an adjuvant.

129. A method for treating a subject for cancer, the method comprising administering to the subject an effective amount of (I) the antibody of any of claims 96-113, (II) the pharmaceutical composition of any of claims 116-121, or (III) the composition of any of claims 122-127.

130. The method of claim 129, wherein the cancer is esophageal squamous cell carcinoma, breast cancer, ovarian cancer, lung cancer, cervical cancer, bladder cancer, osteosarcoma, pancreatic cancers, cholangiocarcinoma, renal cell carcinoma, hepatocellular carcinoma, adrenal cortical carcinoma, glioblastoma, AMK, ANKL, ALL, CLL, or NHL.

131. The method of claim 129, wherein the cancer is MM.

132. The method of claim 130, wherein the cancer is NHL.

133. The method any of claims 129-132, further comprising administering an additional cancer therapy to the subject.

134. The method of claim 133, wherein the additional cancer therapy is radiotherapy, chemotherapy, or immunotherapy.

135. A method for preventing cancer, the method comprising administering to a subject an effective amount of (I) the antibody of any of claims 96-113, (11) the pharmaceutical composition of any of claims 116-121, or (III) the composition of any of claims 122-127.

136. The method of claim 135, wherein the cancer is wherein the cancer is esophageal squamous cell carcinoma, breast cancer, ovarian cancer, lung cancer, cervical cancer, bladder cancer, osteosarcoma, pancreatic cancers, cholangiocarcinoma, renal cell carcinoma, hepatocellular carcinoma, adrenal cortical carcinoma, glioblastoma, AMK, ANKL, ALL, CLL, or NHL.

137. The method of claim 136, wherein the cancer is NHL.

138. The method of claim 135, wherein the cancer is MM.

139. A method of diagnosing a subject for cancer comprising providing to the subject the antibody of any of claims 96-113 and a diagnostic agent.

140. The method of claim 139, wherein the diagnostic agent is a dye.

141. A kit comprising (i) the antibody of any of claims 96-113 and (ii) instructions for use for detecting TfR1 in a biological sample.

142. An antibody comprising:
(a) a V_{H} region comprising SEQ ID NO:7;
(b) a V_{L} region comprising SEQ ID NO:8; and
(c) a C_{H} region comprising SEQ ID NO:9.

143. The antibody of claim 142, wherein the antibody comprises SEQ ID NO:27 and SEQ ID NO:28.

144. An antibody comprising:
(a) a V_{H} region comprising SEQ ID NO:17;
(b) a V_{L} region comprising SEQ ID NO:18; and
(c) a C_{H} region comprising SEQ ID NO:9.

145. The antibody of claim 144, wherein the antibody comprises SEQ ID NO:29 and SEQ ID NO:30.

146. An antibody comprising:
(a) a V_{H} region comprising SEQ ID NO:25;
(b) a V_{L} region comprising SEQ ID NO:26; and
(c) a C_{H} region comprising SEQ ID NO:9.

147. The antibody of claim 146, wherein the antibody comprises SEQ ID NO:31 and SEQ ID NO:32.


SEQUENCE LISTING (32 sequences)

SEQ ID NO: 1 - protein, 5 aa, Artificial Sequence
  Artificial Polypeptide
NYWIE

SEQ ID NO: 2 - protein, 18 aa, Artificial Sequence
  Artificial Polypeptide
ILPGTGRTIYNEKFKGKA

SEQ ID NO: 3 - protein, 13 aa, Artificial Sequence
  Artificial Polypeptide
RDYYGNFYYAMDY

SEQ ID NO: 4 - protein, 10 aa, Artificial Sequence
  Artificial Polypeptide
ASQGINNYLN

SEQ ID NO: 5 - protein, 6 aa, Artificial Sequence
  Artificial Polypeptide
TSTLQS

SEQ ID NO: 6 - protein, 10 aa, Artificial Sequence
  Artificial Polypeptide
QQYSKLPRTF

SEQ ID NO: 7 - protein, 122 aa, Artificial Sequence
  Artificial Polypeptide
QVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSS

SEQ ID NO: 8 - protein, 107 aa, Artificial Sequence
  Artificial Polypeptide
DIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIK

SEQ ID NO: 9 - protein, 255 aa, Artificial Sequence
  Artificial Polypeptide
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTW

SEQ ID NO: 10 - protein, 107 aa, Artificial Sequence [sequence omitted]

SEQ ID NO: 11 - protein, 5 aa, Artificial Sequence
  Artificial Polypeptide
SFAMS

SEQ ID NO: 12 - protein, 17 aa, Artificial Sequence
  Artificial Polypeptide
AISGSGGGTYYADSVKG

SEQ ID NO: 13 - protein, 13 aa, Artificial Sequence
  Artificial Polypeptide
DKILWFGEPVFDY

SEQ ID NO: 14 - protein, 11 aa, Artificial Sequence
  Artificial Polypeptide
RASQSVSSYLA

SEQ ID NO: 15 - protein, 7 aa, Artificial Sequence
  Artificial Polypeptide
DASNRAT

SEQ ID NO: 16 - protein, 10 aa, Artificial Sequence
  Artificial Polypeptide
QQRSNWPPTF

SEQ ID NO: 17 - protein, 122 aa, Artificial Sequence [sequence omitted]

SEQ ID NO: 18 - protein, 107 aa, Artificial Sequence [sequence omitted]

SEQ ID NO: 19 - protein, 10 aa, Artificial Sequence
  Artificial Polypeptide
GYSFTGYTMN

SEQ ID NO: 20 - protein, 17 aa, Artificial Sequence
  Artificial Polypeptide
RINPHNGGTDYNQKFKD

SEQ ID NO: 21 - protein, 9 aa, Artificial Sequence
  Artificial Polypeptide
GYYYYSLDY

SEQ ID NO: 22 - protein, 10 aa, Artificial Sequence
  Artificial Polypeptide
SASSSIRYIH

SEQ ID NO: 23 - protein, 11 aa, Artificial Sequence
  Artificial Polypeptide
DTSNLASGVPA

SEQ ID NO: 24 - protein, 8 aa, Artificial Sequence
  Artificial Polypeptide
HQRNSYPW

SEQ ID NO: 25 - protein, 118 aa, Artificial Sequence
  Artificial Polypeptide
EVQLQQSGPELVKPGASMKISCKASGYSFTGYTMNWVKQSHGENLEWIGRINPHNGGTDYNQKFKDKAPLTVDKSSNTAYMELLSLTSGDSAVYYCARGYYYYSLDYWGQGTSVTVSS

SEQ ID NO: 26 - protein, 106 aa, Artificial Sequence
  Artificial Polypeptide
QIVLTQSPAIMSVSPGEKVTMTCSASSSIRYIHWYQQRPGTSPKRWIYDTSNLASGVPARFSGSGSGTSYSLTISSMEAEDAATYYCHQRNSYPWTFGGGTRLEIR

SEQ ID NO: 27 - protein, 550 aa, Artificial Sequence
  Artificial Polypeptide
QVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 28 - protein, 214 aa, Artificial Sequence
  Artificial Polypeptide
DIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 550 aa, Artificial Sequence
  Artificial Polypeptide
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 30 - protein, 214 aa, Artificial Sequence
  Artificial Polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 546 aa, Artificial Sequence
  Artificial Polypeptide
EVQLQQSGPELVKPGASMKISCKASGYSFTGYTMNWVKQSHGENLEWIGRINPHNGGTDYNQKFKDKAPLTVDKSSNTAYMELLSLTSGDSAVYYCARGYYYYSLDYWGQGTSVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 32 - protein, 213 aa, Artificial Sequence
  Artificial Polypeptide
QIVLTQSPAIMSVSPGEKVTMTCSASSSIRYIHWYQQRPGTSPKRWIYDTSNLASGVPARFSGSGSGTSYSLTISSMEAEDAATYYCHQRNSYPWTFGGGTRLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC